(19) United States
(12) Patent Application Publication
(10) Pub. No.: US 2025/0201380 A1
(43) Pub. Date: Jun. 19, 2025

(54) CONTINUOUS MONITORING SYSTEMS FOR PATIENT PALATAL EXPANSION TREATMENTS

(71) Applicant: Align Technology, Inc., San Jose, CA (US)

(72) Inventors: Jeremy Riley, Mountain View, CA (US); Jun Sato, San Jose, CA (US); Ryan Kimura, San Jose, CA (US); Christopher E. Cramer, Durham, NC (US); Michael Austin Brown, Orlando, FL (US)

(21) Appl. No.: 18/982,401

(22) Filed: Dec. 16, 2024

Related U.S. Application Data
(60) Provisional application No. 63/645,802, filed on May 10, 2024.
(60) Provisional application No. 63/611,770, filed on Dec. 18, 2023.

(51) Int. Cl. G16H 20/40; G06T 7/00; G06T 7/10

(52) U.S. Cl. CPC G16H 20/40; G06T 7/0012; G06T 7/10; G06T 2207/20081; G06T 2207/30036

(57) ABSTRACT

A method for monitoring palatal expansion is provided. The method includes accessing a treatment plan for a patient comprising a series of sequential treatment stages, each treatment stage associated with a particular palatal expander in a series of palatal expanders, receiving 2D images of an oral cavity of the patient during a treatment stage of the treatment plan, determining, via processing of the 2D images, one or more observations progress indicators associated with the treatment plan, determining, based on the one or more observations progress indicators, a level of progress associated with the treatment plan, and providing a representation of progress corresponding to the determined level of progress.

### RELATED APPLICATIONS

This patent application claims the benefit under 35 U.S.C. § 119 (e) of U.S. Provisional Application No. 63/611,770, filed Dec. 18, 2023, and of U.S. Provisional Application No. 63/645,802, filed May 10, 2024, both of which are incorporated by reference herein.

### TECHNICAL FIELD

The instant specification generally relates to systems and methods for holistically monitoring a palatal expansion treatment plan, and in particular to systems and methods for monitoring palatal expansion based on sensor data from sensors of a palatal expander and/or image data.

### BACKGROUND

Treatment plans encompass comprehensive outlines of actions aimed at managing medical conditions. They are often developed by healthcare providers in consultation with the patient and typically include diagnostic tests, therapies, and other interventions that are tailored for an individual patient. Treatment plans and strategies function as “roadmaps” for both healthcare providers and the patients, outlining steps and sequence needed to be taken to achieve the desired health outcomes.

A palatal expansion treatment plan is a type of treatment plan. In pediatric dentistry, palatal expansion can be used to help treat and/or prevent malocclusion by widening the upper arch, creating more space, and addressing posterior crossbite. It is estimated that 10% of children require palatal expansion.

### SUMMARY

The below summary is a simplified summary of the disclosure in order to provide a basic understanding of some aspects of the disclosure. This summary is not an extensive overview of the disclosure. It is intended neither to identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In a first example implementation, a method for monitoring palatal expansion includes: accessing a treatment plan for a patient comprising a series of sequential treatment stages, each of a plurality of the series of sequential treatment stages associated with a particular palatal expander in a series of palatal expanders; receiving one or more two-dimensional (2D) images of an oral cavity of the patient during a treatment stage of the treatment plan; determining, via processing of the one or more 2D images, one or more observations of the oral cavity; determining, based on the one or more observations, a level of progress associated with the treatment plan; and providing a representation of progress corresponding to the determined level of progress.

In a second example implementation, a method for monitoring palatal expansion includes: receiving sensor data generated by one or more integrated sensors of a palatal expander manufactured for a patient; determining, via processing of the sensor data, one or more observations associated with the palatal expander; and providing a representation of progress of the palatal expansion based on the one or more observations.

In a third example implementation, a palatal expander comprises: a first tooth engagement region configured to secure the palatal expander to one or more first teeth of a patient; a second tooth engagement region configured to secure the palatal expander to one or more second teeth of the patient; a palatal region connecting the first engagement region and the second tooth engagement region, the palatal region configured to apply a lateral force across a palate of a patient when the first tooth engagement region is secured to the one or more first teeth and the second tooth engagement region are secured to the one or more second teeth; and; and an integrated sensor configured to sense one or more properties associated with the palate of the patient.

In a fourth example implementation, a method for monitoring palatal expansion includes: accessing a treatment plan for a patient comprising a series of sequential treatment stages, each treatment stage associated with a particular palatal expander in a series of palatal expanders; capturing sensor data associated with the treatment plan from a palatal area of the patient; extracting, via processing of the sensor data, one or more observations associated with the treatment plan; determining, based on the one or more observations, a level of progress associated with the treatment plan; and providing a representation of progress based on the one or more observations.

In a sixth example implementation, a method comprises: receiving one or more images of a current face of a patient at an intermediate stage of a dental treatment plan; generating one or more simulated images of a post-treatment face of the patient based at least in part on processing of the one or more images of the current face of the patient; determining one or more observations of the post-treatment face of the patient in the one or more simulated images; and determining, based on the one or more observations, whether to adjust the dental treatment plan.

### BRIEF DESCRIPTION OF THE DRAWINGS

Aspects and embodiments of the present disclosure will be understood more fully from the detailed description given below and from the accompanying drawings of various aspects and embodiments of the disclosure, which, however, should not be taken to limit the disclosure to the specific aspects or embodiments, but are for explanation and understanding only.

FIG. 1 illustrates an example palatal expansion treatment plan, according to some embodiments of the present disclosure.

FIGS. 2A-B illustrate example palatal expanders for use during the palatal expansion treatment plan of FIG. 1, according to some embodiments of the present disclosure.

FIG. 3 illustrates an example monitoring process for use during the palatal expansion treatment plan of FIG. 1, according to some embodiments of the present disclosure.

FIG. 4 illustrates an example dataset generation process for use with data captured from the palatal expansion treatment plan of FIG. 1, in accordance with some embodiments of the present disclosure.

FIG. 5 illustrates an example training process for training an analysis module via the dataset of FIG. 4, in accordance with some embodiments of the present disclosure.

FIG. 6 illustrates an example customization process for customizing the palatal expansion treatment plan of FIG. 1, in accordance with some embodiments of the present disclosure.

FIG. 7A illustrates a flow diagram of an example method for monitoring palatal expansion progress, in accordance with some embodiments of the present disclosure.

FIG. 7B illustrates a flow diagram of an example method for generating measurements used to monitor palatal expansion progress, in accordance with some embodiments of the present disclosure.

FIG. 7C illustrates a flow diagram of an example method for determining palatal expansion progress based on measurements associated with a palatine rugae, in accordance with some embodiments of the present disclosure.

FIG. 7D illustrates a flow diagram of an example method for determining palatal expansion progress based on measurements associated with a palatine rugae, in accordance with some embodiments of the present disclosure.

FIG. 7E illustrates a flow diagram of an example method for determining palatal expansion progress based on measurements associated with a dental arch width, in accordance with some embodiments of the present disclosure.

FIG. 8A illustrates a flow diagram of an example method for identifying adverse effects associated with palatal expansion, in accordance with some embodiments of the present disclosure.

FIG. 8B illustrates a flow diagram of an example method for predicting a post-treatment face of a patient associated with palatal expansion and/or orthodontic treatment, in accordance with some embodiments of the present disclosure.

FIG. 9 illustrates a flow diagram of an example method for determining whether a palatal expander is worn correctly, in accordance with some embodiments of the present disclosure.

FIG. 10 illustrates a flow diagram of an example method for monitoring a palatal expansion based on sensor measurements, in accordance with some embodiments of the present disclosure.

FIG. 11 illustrates a flow diagram of an example method for determining how to adjust a treatment plan based on sensor data associated with transverse force on a palatal expander, in accordance with some embodiments of the present disclosure.

FIG. 12A illustrates an image of an upper dental arch of a patient prior to palatal expansion treatment, according to certain embodiments.

FIG. 12B illustrates an image of an upper dental arch of a patient during a stage of palatal expansion treatment, according to certain embodiments.

FIG. 13A illustrates a series of palatal expanders that are configured to progressively expand the suture, according to certain embodiments.

FIG. 13B illustrates a passive holder (e.g., retainer) that can be worn, for example, after the series of FIG. 13A has completed expanding the patient's palate, according to certain embodiments.

FIG. 14 illustrates an example system architecture capable of supporting holistic monitoring of a palatal expansion treatment plan, in accordance with one embodiment of the present disclosure.

FIG. 15 shows a block diagram of an example system for virtual dental care associated with a palatal expansion treatment, in accordance with some embodiments.

FIG. 16 depicts a block diagram of an example processing device operating in accordance with one or more embodiments of the present disclosure.

### DETAILED DESCRIPTION

Dental treatment plans (e.g., including orthodontic treatment plans, palatal expansion treatment plans, etc.) often include multi-stage plans that are broken down into various phases or stages. Each stage has its own set of specific procedures and goals, which are designed to address different aspects of the dental issue at hand. For example, an orthodontic treatment plan for palatal expansion (or that includes a palatal expansion component) can start with an initial stage focused on initial expansion of the palate, followed by a second stage aimed at stabilizing bone growth, and other stages, as necessary. These stages can be further granularized to enhance precision and targeting in the treatment plan.

In some cases, such a dental treatment plan can encompass the use of hardware or device(s) that are specifically modified or interchanged at each phase or stage of the treatment. For example, with respect to palatal expansion, palatal expanders (PEs) are devices custom-fitted to a pediatric patient's upper jaw that are used to incrementally widen a patient's palate. In one implementation, a custom-fit palatal expander device is worn by a patient. The palatal expander device is then incrementally adjusted, potentially on a daily basis. Such adjustments can be performed to gradually widen the upper jaw. In some cases, these adjustments can be made through mechanisms like a set screw. In some cases, instead of adjusting the device, a new palatal expander device can be switched with the current one. In either case, a new stage of the treatment plan can call for the replacement of one type of palatal expander with another, depending on the progress and specific needs of the patient's jaw development.

Such staged palatal expansion treatment plans can further include scheduled stages or checkpoints at timepoints within, or between, one or more treatment stages. Such checkpoints are junctures where a dental practitioner evaluates the progress of the treatment and decides whether it is appropriate to move on to the subsequent stage of treatment. Regularly scheduled checkpoints provide an opportunity for re-evaluation and, if necessary, modification of the treatment plan. This ensures that the treatment is on the right track and allows for course corrections, enhancing the likelihood of a successful outcome.

Traditionally, at each checkpoint a patient needs to visit their doctor, who examines the patient's dentition to determine whether the palatal expansion and/or orthodontic treatment is progressing as planned. If palatal expansion is not progressing as planned (e.g., a patient's palate is expanding slower than anticipated or faster than anticipated, a patient's teeth are tipping, etc., the doctor then needs to determine how to adjust the treatment plan. Such patient visits and doctor evaluations take time for the patient and for the doctor.

Aspects and implementations of the present disclosure address the above and other challenges by providing systems and methods for virtual monitoring of palatal expansion treatment and/or orthodontic treatment based on images of a patient's upper dental arch (e.g., based on photos periodically taken by the patient and uploaded to a server and/or based on sensor data as measured by sensors of the palatal expander) and/or based on sensor data from sensors integrated into the palatal expander(s). The images may be analyzed to determine whether a patient's dental arch width is increasing as anticipated, for example. Sensor data may additionally or alternatively indicate whether a force or pressure profile as measured by force/pressure sensors of the palatal expander indicate that palatal expansion is progressing as planned. Based on the image data and/or sensor data, processing logic may automatically determine whether palatal expansion treatment is progressing as planned and/or whether adjustments should be made to the treatment plan. Such adjustments may be automatically determined in embodiments, and may be presented to a doctor for approval prior to updating the treatment plan. Accordingly, in embodiments palatal expansion may be virtually monitored without requiring a patient to visit their doctor (e.g., dentist or orthodontist).

In some implementations, one or more 2D images are captured of a patient's face during one or more stages of treatment. The 2D image(s) of the patient's face may be processed to generate a synthetic image showing a predicted post-treatment face of the patient. One or more observations may be made about the post-treatment face of the patient, such as observations about a face width, a nasal breadth, a facial asymmetry, and so on. The observations made about the post-treatment face may be used to determine one or more adjustments to a palatal expansion treatment plan and/or an orthodontic treatment plan in embodiments.

In some embodiments, palatal expanders (PE(s) and an accompanying system is used which is capable of replacing a traditional palatal expander. The proposed PE(s) can include a set of custom-printed devices that can be staged to widen the arch, rather than requiring a single bonded device where a screw must be turned to widen the arch.

In embodiments, the PE(s) and system are capable of remote, photo-based and/or sensor-based approaches to holistically monitor the progress of palatal expansion. In some cases, such a system and associated palatal expansion treatment plan can forgo regular visits to an orthodontist. In embodiments, such holistic, remote monitoring can help to ensure that the device staging does not outpace physiological changes. Additionally, holistic, remote monitoring can help to ensure that the observed arch expansion is skeletal and not just dental; and can identify key events in the palatal expansion (e.g., such as the opening of a diastema between the patient's upper central incisors).

In embodiments, various methods of monitoring palatable expansion can be used. For instance, during expansion stage(s), features or elements indicative of expansion progress can be monitored. During retention stage(s) (e.g., after a palatal expansion stage), similar, or additional features can be monitored. Throughout both stages, the occurrence of adverse events can be constantly or periodically monitored. Such monitoring approaches can be referred to herein as progress monitoring, retention monitoring, and event monitoring, respectively. In general, such monitoring can be referred to herein as holistic monitoring. Thus, monitoring of a patient throughout a palatal expansion treatment plan can be comprehensive or holistic, and encompass various stages, sensors, events, metrics, parameters, and so on. Similar monitoring may also be performed during orthodontic treatment.

Progress monitoring can be used to ascertain the rate at which palatal expansions is being achieved, i.e., if a patient is still progressing along the treatment goals or if progress has stopped or slowed. Progress monitoring can also indicate to the doctor that certain treatment goals are met. Progress monitoring can be used as an input to related monitoring techniques. In some cases, progress monitoring can inform a patient when certain stage-goals have been achieved, and/or when a patient can advance to a subsequent stage (and/or PE).

Multiple approaches are proposed for progress monitoring. For instance, arch width may be monitored, posterior crossbite may be monitored, diastema may be monitored, palatine rugae may be monitored, device fit may be monitored, facial symmetry may be monitored, face width may be monitored, nose breadth may be monitored, and so on. In embodiments, a photo-based, or image processing, approach may be used to monitor such aspects of a palatal expansion treatment plan. In embodiments, embedded sensors within a palatal expander may be used.

To monitor arch width of a patient (e.g., during progress monitoring), an upper occlusal image and the patient's treatment plan can be used. The occlusal image can first be segmented into the individual teeth and/or other dental structures, e.g., by processing the image using a machine learning model trained to perform image segmentation of intraoral images. To determine the arch width between a set of teeth (e.g., such as the first molars or the canines), the physical size of a pixel may be useful. To achieve such, a known physical width of a feature (e.g., the relevant arch width, tooth width, etc.) from the treatment plan, e.g., in mm, can be compared to the corresponding size in an image, e.g., in pixels. In order to determine the size of the pixels in that region of the image, a pixel size can be computed based on dividing the image pixel width of the feature by the known physical size. For instance, when using a tooth as a reference, the number of pixels from the buccal edge of the left and right instances of the relevant tooth can be measured. The total arch width at the relevant tooth is then the pixel distance multiplied by the pixel size. In addition to, or instead of, width of the known teeth, other features can be used to determine sizes. For example, size of teeth in all dimensions, size of the palatal rugae, etc. can be used to compute pixel sizes.

An additional, or alternative, method to determine pixel size can use the registration of the expected 3D model of the upper arch to the image. Such an approach may be used to estimate an angle of the camera to the arch, and to compute a perspective-correction factor for improving the pixel size estimate, the arch width measurement, or both.

In embodiments, an additional key malocclusion that may be monitored during the palatal expansion treatment plan (e.g., during progress monitoring) is posterior crossbite. Posterior crossbite can occur when the occlusion of the buccal cusps of the upper molars is on the central fossae of their opposing lowers (as opposed to the buccal cusps of the lower molars occluding between the buccal and lingual cusps of the upper molars). The proposed palatal expander, in embodiments, may correct posterior crossbite. The correction of posterior crossbite may be an indication that the expansion has sufficiently widened the arch and that further staging may not be required. To monitor posterior crossbite, usage of either an anterior image or, preferably, one or more lateral images, is proposed. These images can be taken by a patient (or another individual such as patient's family member), and be provided to an HCP via a virtual care system.

During use of an anterior image, the system can first segment the image into individual teeth and/or other dental structures (e.g., using a trained machine learning model), and compare a buccal edge of the maxillary molars to the buccal edge of the mandibular molars once the image has been segmented. Where the mandibular molars are more buccal than the maxillary molars, the patient may be exhibiting posterior crossbite. When the maxillary molars are more buccal than the mandibular molars, the crossbite may have been corrected. Where a single, closed-bite, lateral image is available, a different method can be used. The teeth can be segmented in the image, and the heights of the maxillary and mandibular molars (in pixels) can be found.

Skeletal expansion of the palate can often be accompanied by the creation and/or widening of a diastema between the maxillary central incisors. This can be monitored (e.g., during monitoring of adverse events). For instance, in embodiments, the virtual care system can monitor the existence and/or size of the diastema using an anterior image, which can be an open bite image or closed bite image. This image can be segmented into individual teeth and/or other dental structures, and the distance between the left and right central incisors can be measured in pixels. A pixel size can be found (e.g., as described above) and the distance (if any exists) can be converted to a physical unit of measurement, such as millimeters. Such an approach can also be used in monitoring an orthodontic treatment intended to close a diastema.

Similarly, skeletal expansion can also be accompanied by changes to the palatine rugae, which can be monitored e.g., during progress monitoring. By monitoring the rugae, the proposed virtual care system can differentiate between palatal widening and dental expansion (i.e., determine if the palate is widening or if the dentition is moving/tilting). To assess the rugae, an upper occlusal image can be processed by the system. The outlines and/or contours of the rugae can first be identified and defined (e.g., using segmentation, edge detection, contour detection, color references, etc.). After, the rugae contours can be aligned to an initial, or previous, set of rugae contours. Once the current rugae contours have been aligned to the initial or previous set of contours, the contour length, distance between contours, contour shape, distance between a point on one or more palatine rugae and one or more other oral features, etc. can be compared between the current and prior images. Contour distance (distance between two contours of the palatine rugae) can increase with the amount of palatal (skeletal) expansion, and can remain constant should expansion be dental, as opposed to skeletal, for example.

Multiple approaches can be used to align rugae contours. Such approaches include identifying the incisive papilla (e.g., through object detection) and using it as a reference point to align the rugae contours. Other approaches can include computing and/or optimizing a transform that best aligns the current and previous and/or initial rugae contours. In one embodiment, homography is used for the transform. The transform can also be an affine, or other, transform. The transformation can be found using matching points or by optimizing over a distance metric between the two or more rugae contours. A variety of distance metrics can be used, both for assessing the change in rugae and for computing an optimal transform. In one embodiment, a Hausdorff distance or a modified (one-way) Hausdorff distance can be found. Other options include identifying matching points (e.g., using biological reference points or image processing techniques such as SIFT, SURF, or ORB) and computing a mean distance between the matching points. In some embodiments, a subset of the rugae contours can be used to optimize the transform between the images (e.g., only those lying on a particular plane). Additional variations include using a subset of the rugae contours to measure the contour distance.

Another approach to monitoring treatment (e.g., during progress monitoring) can be to monitor the fit of the palatal expander as the patient wears it. In such, images of a patient wearing the palatal expansion device can be collected. From such, fit issues such as gaps between occlusal surfaces of the molars and device can be detected. One potential cause of a device that is not fitting may be a missing retention attachment.

In some embodiments one or more images of the patient wearing the palatal expansion device (i.e., palatal expander) are processed to determine a state of the palatal expansion device. The palatal expansion device may become degraded or damaged from wear based on a patient biting, grinding their teeth, etc., while wearing the palatal expansion device. In embodiments, the one or more images of the patient wearing the palatal expansion device may be processed to determine whether the palatal expansion device has experienced excessive wear. Such an assessment may be made, for example, by processing the image(s) using a trained machine learning model that may output an indication of an amount of wear of the palatal expansion device and/or an indication of whether the palatal expansion device has experienced excessive wear. If the palatal expansion device has experienced excessive wear, then a notification to replace the palatal expansion device may be generated in embodiments.

As discussed, during the course of a palatal expansion treatment, several adverse or unusual events can occur. These events should be brought to a doctor's attention, and may occur at any point or stage of the palatal expansion treatment plan. By way of example, such events can include soft tissue damage, intrusion of supporting teeth, excessive buccal tipping of the molars, anterior or posterior tissue impingement, tooth eruption or exfoliation, and so on. Such issues may be identified based on analysis of 2D images taken of the upper palate of the patient during treatment.

Soft tissue damage can be detected from one or more of an occlusal, anterior, and/or lateral image. To begin, object detection and/or classification can identify images or regions of images exhibiting soft tissue damage such as ulceration on the gingiva, palatal surface or buccal mucosa. Ulceration on the gingiva may be apparent in lateral images. Ulceration of the palatal surface may be apparent from occlusal images, and ulceration of the buccal mucosa can be visible on anterior images. In embodiments, object detection and classification to accomplish such can be based on various factors, ranging from simple color deviations to more complicated computations. In some cases, such can be accomplished through trained machine learning models (MLMs) and large numbers of samples.

Intrusion (e.g., from supporting teeth) can be identified by segmenting an image (as described) and measuring the height of the clinical crowns of the intruding teeth in the image. Such a measurement can be performed using pixels and then converted to physical units (e.g., as described).

Buccal tipping of molars can be monitored. Using segmentation of an anterior image (as described), the amount of buccal tipping of the maxillary molars can be found by examining the angle of one or more teeth in the image. The segmentation of a single molar may be indicated by a curved surface that is generally pointing in the positive-y direction (e.g., in image space), but a buccally tipped molar will be less downward pointing. In some embodiments, buccal tipping of molars may be detected using one or more sensors (e.g., accelerometers, gyroscopes and/or rotational sensors) that measure an amount of torque on the one or more molars.

Tissue impingement can be found in the image as a line at the posterior of the palate. Such may be detectable in an occlusal image. Detection can be performed by means of object detection, semantic segmentation, and/or standard edge detection in the image (as described).

In embodiments, eruption of missing teeth and/or exfoliation of current teeth may occur throughout a palatal expansion treatment plan. This may be detected through image segmentation from any of the standard intraoral images. Exfoliation may be detected as a tooth that is no longer present in image data although it remains present in the treatment plan. Eruption can be detected as a “new” tooth in the image without a corresponding tooth in the treatment plan. In addition to direct segmentation, eruption and/or exfoliation can be identified by comparing an initial or previous image to the current image, potentially through the use of an image transformation, as described above regarding changes in the palatal rugae.

Once an expansion has been completed, the patient may be put into a retention stage, where the expansion gains are consolidated. During the retention stage, several features and/or aspects of a patient's dentition can be monitored (i.e., during retention monitoring). For instance, in embodiments, a patient's arch width, molar tipping, aspects of the retention device, etc. can be monitored.

Arch width retention can be monitored during a retention stage using similar techniques to those summarized above with reference to palatal expansion stages. Molar tipping can be monitored during a retention stage. For instance, molar tipping can occur if there is a relapse of the palatal expansion, but the retention device is holding the molars in place. This can be monitored in the same way that the excessive buccal tipping of molars is monitored (e.g., in the adverse and/or unusual event monitoring described above).

Discoloration of the retention device can be monitored in a retention stage and/or palatal expansion stage. Discoloration can indicate that the device is being stressed or strained in an unexpected manner. In an example, by comparing the color of a retention device in a current occlusal image with the retention device on an older or initial image, color changes can be detected. Image color and white balance may play a role in the absolute color of the device. Image processing logic can establish a color comparison, e.g., by using the tooth color and/or the gingival color as a reference. By aligning the reference colors to be equivalent, image processing logic can compare the device color. An alternative approach is to use a portion of the device that is less subject to discoloration as the reference color for computing the color balance transformation. When the device seems to be changing colors, there is an indication that the patient's expansion is relapsing. Note that such an approach to comparing color can also be used during progress monitoring to identify unusual stresses or strains on the device. Such forces can imply that staging may be outpacing skeletal changes of the patient, and that the staging of the palatal expansion treatment plan should be slowed.

In some embodiments, the above monitoring mechanisms can be enabled for palatal expander by the use of electronic sensors (e.g., force sensor, pressure sensor, displacement sensor, etc.) embedded on or within the palatal expander. Sensors can be used for two main categories of actions in embodiments. First, sensor data may be used during treatment of an individual patient to make live adjustments to that patient's treatment plan (e.g., to perform progress monitoring, event monitoring, retention monitoring). In some embodiments, through holistic monitoring, a feedback loop for adjusting a treatment plan can be provided. Second, data from many patients can be analyzed and personalized treatment plans can be developed in advance for future patients.

A force sensor (or pressure sensor) within the palatal region of the palatal expander can be used to measure force build-up. It should be understood that any discussion of force sensors herein also applies to pressure sensors. Additionally, any discussion of pressure sensors should be understood to also apply to force sensors. If force builds-up from stage to stage to a sufficient level, then the patient should stay in their current expander until the force drops. If this happens multiple times, the rate of expansion can be slowed down for that patient. Note that any discussion herein with reference to force sensors applies equally to pressure sensors. Accordingly, it should be understood that embodiments relating to the use of force sensors may equally be applied with the user of pressure sensors.

A force sensor or pressure sensor within the palatal region of the palatal expander can be used to measure force reduction during the holding period. Once the force drops to a low enough level, the patient can proceed to the next stage of treatment, such as a stage 1 to 2 retainer, aligners, or other treatment.

A force sensor or pressure sensor within the buccal region of the palatal expander can be used to measure the force to insert or remove the palatal expander from an upper dental arch of a patient. Should insertion or removal force be excessively high, a reminder can be sent to the patient on the insertion and removal technique.

A displacement sensor on the occlusal region of the palatal expander can be used to assess fit of the palatal expander. If the palatal expander is not fully seated, the patient can be instructed to use chewies to fully seat the palatal expander on the dentition.

A colorimetric sensor on the palatal expander can be used to assess staining on the device and encourage the patient to avoid certain high-staining-risk foods or drinks.

A biofilm sensor on the palatal expander can be used to assess biofilm build-up on the device and encourage the patient to avoid certain high-biofilm risk foods or drinks.

A temperature sensor on the palatal expander can be used to detect if the patient is consuming excessively hot food or drink which can negatively affect the function of the device.

A rotational sensor in a palatal region of the palatal expander can be used to detect rotation of one region of the palate relative to another region of the palate (e.g., of a left side of the palate relative to a right side of the palate). Such rotation of the palate may be caused by the palatal expansion in embodiments.

One or more rotational sensors in tooth regions of the palatal expander can be used to detect rotation of teeth (e.g., of molars). Such detected rotation may be separate from rotation of the palatal regions relative to one another, and may indicate tooth tipping in embodiments.

One or more force (or pressure) sensors may be disposed in one or more regions (e.g., tooth regions) of the palatal expander, and may be used to detect bite force on the palatal expander. Measurements of bite force may be used to detect how many bites the palatal expander has been subjected to, whether the patient is experiencing bruxism, whether the patient grinds/clenches their teeth at night, etc. Additionally, bite force may be measured at multiple different locations (e.g., at multiple different teeth), and such data may be used to perform a bite analysis (e.g., to determine bite contact points). Improper bite contact points may cause temporomandibular joint disfunction (TMD), jaw pain, and so on. In embodiments, palatal expanders as well or orthodontic appliances may be modified to change bite contact points and improve a patient bite during treatment. An output of the bite analysis may be output to a display of, for example, an output device such as a patient device or a doctor device.

As discussed, sensor data can be used to inform changes to the treatment plan (e.g., for determining midcourse corrections). The sensor data can be used to capture milestones (e.g., suture breakage), fit, progress tracking, and so on. For example, a dental practitioner may want to keep forces within an ideal range over the course of the entire treatment plan, over particular treatment stages, and so on. Alternatively, a treatment plan can keep the force adhering to particular force profiles within each stage of treatment.

In some embodiments, the feedback loop and sensors can measure insertion and/or removal force of a device for attachment debonding. In one embodiment, if measurements are too high, a next set of palatal expanders can be adjusted to prevent debonding. Such adjustment can also balance forces along the attachments. Such measurements can also be used to suggest rebonding if the attachment is causing an issue with retention and/or with engagement.

In the case where measurements indicate inadequate levels of biting, clenching, and/or fitting, an action can be recommended, such as recommending use of a chewie to the patient. This can also prompt course correction and/or plan modification. For example, in embodiments, a force sensor (or any other suitable sensor) can be used around the crown region for determining when the palatal expander is fully engaged. In embodiments, this can track engagement over time, and can send a notification (e.g., “your palatal expander is not fully engaged”) to a patient and/or HCP. Such a sensor can be used to send additional data to the patient and/or HCP. For instance, such a sensor can be used to alert when expansion force exceeds a maximum allowable threshold. This can also include alerts for when an attachment is debonded or is likely to be debonded.

In embodiments, force balancing can be implemented. When implementing such, the system can have sensors on each of the three segments and/or bands of a palatal expander and make sure the force is balanced.

In some embodiments, a data-driven, personalized treatment mechanism can be provided. In some embodiments, based on known parameters about a given patient (age, gender, arch width, crown height, palatal vault depth, etc.) the design of the palatal expander or attachments, or the treatment plan (rate of expansion, etc.), can be customized based on data collected and analyzed from previous patients using palatal expander with sensors. For example, in younger patients, the suture can be more compliant, and less force can be required. This information may be used in treatment planning to determine an amount of palatal expansion to be applied at each stage of treatment, a number of stages of treatment to use, and so on.

The forces and movement experienced by patients can vary from patient to patient (e.g., based on demographics such as biological age, dental age, sex, ethnicity, genetic profiles). Accordingly, it may not be ideal to interpret sensor data uniformly across all patients. For example, in an older patient, tissues can be less compliant and forces can build up more quickly than in a younger patient. Accordingly, a force measurement of X in an older patient can be equivalent to a force measurement of 2X in a younger patient. By calibrating for the individual patient, processing logic can make more accurate assessments about treatment progress and better recommendations for when to move on to the next stage (or when to have a mid-treatment course correction). The calibration can be based on demographics and historical information associated with demographics. In embodiments, the design and aspects of a dental treatment plan can be proactively changed.

In some embodiments, a system can optimize a palatal expansion treatment plan. The system can access a palatal expansion treatment plan for a patient comprising a series of sequential treatment stages, each treatment stage associated with a particular dental appliance in a series of palatal expanders. The system can receive patient data comprising one or more progress indicators associated with the palatal expansion treatment plan. The patient data can include, for example, images of a patient's dentition (e.g., from an intraoral scanner; a smartphone or other camera device), biomarker data indicative of changes in the patient's dentition, pressure data indicative of a level of pressure exerted by the patient's dentition on an orthodontic expander, or data indicative of an electrical parameter associated with a position of a tooth with respect to an orthodontic aligner, and so on.

The system can determine, based on the one or more progress indicators, a level of progress associated with the palatal expansion treatment plan. Based on the determined level of progress, the system can determine a treatment modification for the patient. The system can decide to advance the patient to a subsequent treatment stage in the series of sequential treatment stages before a preplanned advancement time, or to retain the patient in a current stage of the series of sequential treatment stages beyond a preplanned advancement time. The system can generate a notification indicating the determined treatment modification.

In some cases, widening a patient's palate can cause the patient's face to widen or cause the base of the patient's nose to broaden. In some limited cases, it may even introduce an asymmetry to the patient's face. Some patients may not wish for their face to be widened beyond a certain amount, may not wish for their nose breadth to be increased, etc. In embodiments, one or more images of a current face of a patient at an intermediate stage of treatment of a dental treatment plan may be captured and received. The one or more images may be processed (e.g., using a trained machine learning model such as a generative model) to generate one or more simulated images of a post-treatment face of the patient. In embodiments, the one or more current images, one or more prior images of the patient's face, a current treatment stage and/or a total number of treatment stages may be input into a machine learning model, which may generate the one or more simulated images. One or more observations may be determined of the post-treatment face of the patient in the one or more simulated images. A determination may then be made, based on the one or more observations, of whether to adjust the dental treatment plan. For example, a patient may be shown their predicted post-treatment facial appearance, and may make an informed decision as to whether to shorten or stop palatal expansion treatment.

Some embodiments are discussed herein with respect to palatal expanders and a palatal expansion treatment plan. It should be understood that such embodiments also apply to other dental appliances that may be worn by a patient, such as orthodontic aligners. For example, an orthodontic aligner may include any of the sensors described herein, which may be used to assess a state of orthodontic treatment in embodiments.

Aspects and implementations of the present disclosure address the above challenges of palatal expansion treatment plans by providing systems and methods to monitor and further personalize and optimize a palatal expansion treatment plan, while limiting lifestyle invasiveness. The proposed systems and methods provided herein can include an optimization module for analyzing and quantifying a patient's treatment progress, and optimizing the stages of a treatment plan, according to some embodiments of the present disclosure. For instance, in some embodiments, an initial treatment plan can be generated (e.g., based on patient information such as patient demographics, type of treatment required, a patient's particular teeth arrangement, data gathered from a dental professional system used to scan the patient, etc.), after a patient has visited a dentist and/or orthodontist office. One or more palatal expanders can be made for the patient, in accordance with scheduled stages and checkpoints of the generated, initial treatment plan. As the treatment plan is on-going, the patient can use an at-home imaging method (e.g., via use of a smartphone, a case for dental device(s), or a similar data collection device, etc.), to monitor progress at predetermined checkpoints determined in the initial treatment plan. Sensor data may additionally or alternatively be automatically generated by integrated sensors in the palatal expander(s). Data can thus be collected at the checkpoints and/or continuously. For example, electronic sensors (e.g., force sensor, displacement sensor, etc.) embedded on or within palatal expander can capture data indicative of palatal expansion (and tooth or jaw movement), patient compliance data can be captured by sensors that monitor how long the device is being worn, sensors within a dental device can capture force or displacement data, and so on. Afterwards, the optimization system and/or module can determine that the initial treatment plan should be updated based on the data. For example, the patient can be instructed to stay at the current stage for a specified number of days, the patient can be advanced earlier than scheduled to a subsequent stage, the checkpoint intervals can be updated, or a similar modification to the initial plan can be made. Furthermore, based on the captured data, more or less monitoring can be determined as necessary. For example, more monitoring can be planned in cases where a patient is less compliant, more monitoring can be planned for a patient who appears to be presenting with some oral health risk (e.g., gingival recession, potential occlusion issues, potential tooth root fenestration), more monitoring can be planned for a patient whose palate, or teeth, movement is slower than according to plan, etc. Thus, an updated plan and/or updated checkpoint intervals can be generated based on collected data and sent to the patient. Thus, a patient can receive and follow an updated plan including updated checkpoint intervals.

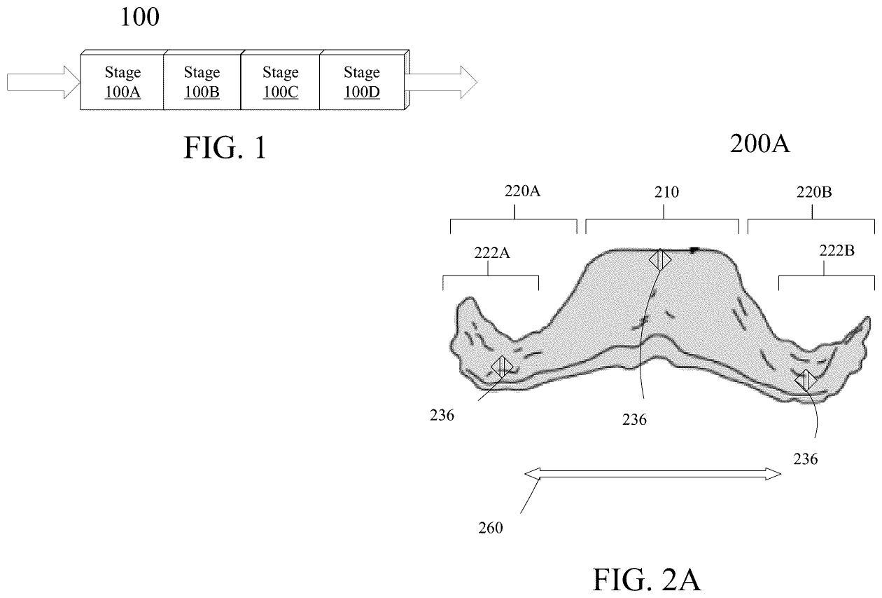

Such a system will be illustratively described throughout the specification set forth below (i.e., with respect to FIGS. 1-11). Furthermore, exemplary embodiments of such a system will follow throughout the specification set forth below (i.e., with respect to FIGS. 1-11). Embodiments described herein provide significant advantages over traditional palatal expansion treatment plans. Such embodiments can provide a system that notifies a health care provider (HCP) and/or patient of a level of progression with respect to a dental treatment plan, notifies an HCP of problems arising during palatal expansion, provides a doctor and/or patient with simulated images of a predicted post-treatment facial appearance, and so on. Such embodiments can provide a system that determines modifications to the palatal expansion treatment plan based on the level of progression and suggest actions to perform to ensure that a planned treatment outcome is achieved. Accordingly, treatment plan efficacy is improved and optimized, in embodiments. Such improvements in treatment plan efficacy are likely to result in increased patient satisfaction as well as reduced costs by reducing the number of consecutive refinements that are made to a treatment plan (and associated orders of additional aligners) during treatment while also minimizing a number of patient visits to an HCP.

Furthermore, some embodiments are discussed herein with reference to orthodontic treatment plans that include generation and use palatal expanders. A palatal expander, in this context, is an orthodontic device specifically designed to widen the upper jaw. In certain implementations, these expanders apply pressure at strategic points across the palate. This pressure is often exerted through components like expansion screws, which can be adjusted to vary the force applied to the palate. The magnitude of each of these pressures and/or their distribution on a patient's upper jaw can determine the type of movement, or palatal expansion, that occurs.

The movements facilitated by palatal expanders can vary in direction and occur in different planes of space, encompassing a range of adjustments. Such adjustments can thus include the widening of the upper jaw, correction of crossbites, improvement of overall jaw alignment, and so on. Such adjustments can be horizontal, focusing on the lateral expansion of the jaw. Such adjustments can lead to a more balanced alignment of the upper and lower jaws.

FIG. 1 illustrates an example treatment plan 100, according to some embodiments of the present disclosure. In some embodiments, treatment plan 100 can be an example initial palatal expansion treatment plan, and can include separate stages 100A-D of the treatment plan. Accordingly, the four stages 100A-D seen in FIG. 1 should be seen as exemplary. One of ordinary skill in the art, having the benefit of this disclosure, will appreciate that numerous stages (e.g., more, or fewer stages than four) can be included in a treatment plan, including an initial treatment plan.

In embodiments, the initial treatment plan 100 can be crafted by an HCP, such as an orthodontist or dentist specialized in orthodontic treatment and/or by treatment planning software. The design of this plan can take into account a multitude of factors, including but not limited to the current state of a patient's palate, a level of desired expansion of the palate, alignment of the patient's teeth, the complexity of the patient case, patient demographics, medical history, the desired end result, etc. Furthermore, specific goals and targets can be associated with each stage of the initial treatment plan, and a dental treatment plan in general. As will be further discussed with respect to FIG. 3, initial diagnostic tools such as X-rays, digital scans, physical molds, virtual 3D models, etc., can be employed by the HCP to generate an initial treatment plan to meet the unique needs of a patient. In alternate embodiments, the initial treatment plan can be entirely, or partially, computer-generated.

In embodiments, the initial dental treatment plan 100 can be divided into multiple stages, serving as a roadmap for the treatment. As an example, stages 100A-D can represent four sequential stages within treatment plan 100. Each stage can correspond to specific goals and targets in the treatment process and can be time-bound. For example, the length of each stage within 100A-D can vary depending on patient case details, treatment goals, patient physiology, methods of therapy, etc. For example, in a treatment plan for a palatal expansion process, each stage can involve a distinct palatal expander to be worn for the duration of a stage. In some cases, one or more stages of the treatment plan can share a palatal expander.

In embodiments, each stage can be progressed through once a target or goal for palatal expansion has been reached for the stage. Thus, in embodiments each stage can be associated with a target or goal of the associated palatal expansion treatment plan. In embodiments, the duration of different stages and/or number of stages can be set based on the difficulty of treatment and/or strategies associated with achieving treatment goals. Thus, in embodiments one or more of the stages can have different durations planned in the initial treatment plan. In alternate cases, duration for all stages can be uniform. As discussed above, in embodiments, the duration of a stage can vary based on treatment complexity. In embodiments, a length of each stage can be anywhere from 1 day, to 3 days, to 10 days, to 3 weeks, in duration.

Continuing with the above example, initial treatment plan 100 can be oriented towards a palatal expansion process, involving the use of a palatal expander (or series of palatal expanders) worn by the patient. The stages 100A-D of the initial palatal expansion treatment plan 100 can involve the use of a sequence of palatal expanders (or a single adjustable palatal expander) to be worn by a patient. Such palatal expanders can exert specific forces that gradually move the palate and/or teeth into a target position. The amount of palatal expansion can be granularized, or discretized, into discrete stages corresponding to stages 100A-D. Subsequent stages (e.g., later stages of the treatment plan, including those not shown in FIG. 1) can continue in a similar manner. These stages can further expand the palate, reposition teeth, and/or introduce further refinements. In embodiments, the subsequent stages 100C-D (and any stages of a treatment process, including those not shown) can introduce further nuanced designs and produce final refinements for palatal expansion and/or orthodontic alignment.

In embodiments, a palatal expander associated with one or more stages of the palatal expansion treatment plan can be 3D printed. In some cases, the palatal expander can be adjustable in width. In some embodiments, the palatal expander can be of a fixed length, and be part of a set of palatal expanders of varying widths. These can be intended to be used in a specific order. In some cases, a palatal expander can be of an adjustable width (e.g., via a set screw, ratcheting mechanism, etc.). An adjustable palatal expander can be used over multiple stages corresponding to multiple widths of the palatal expander. In some instances, a width of the palatal expander can be adjusted to accommodate the changing needs of the patient's palate during the treatment.

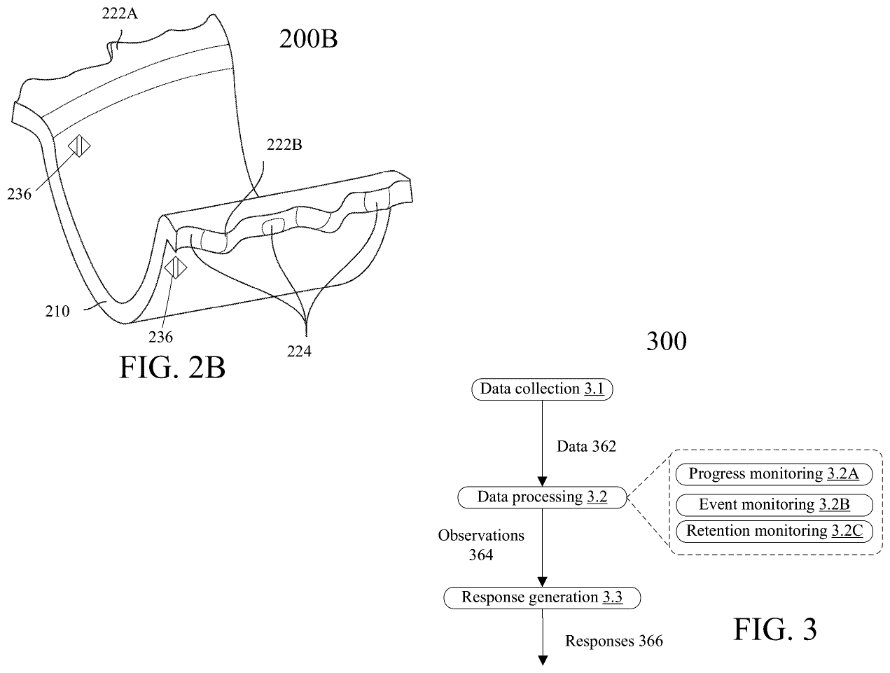

FIG. 2A illustrates an example palatal expander 200A, that can be used during a palatal expansion treatment plan of FIG. 1, according to some embodiments of the present disclosure. FIG. 2B illustrates an additional example palatal expander 200B, that can be used during a palatal expansion treatment plan of FIG. 1, according to some embodiments of the present disclosure.

With reference to FIGS. 2A-B, the structural design of a palatal expander 200A-B can include at least two bilateral arms 220A-B, extending from a palatal region 210. Alternatively, the bilateral arms 220A-B may be omitted (e.g., as shown in FIG. 2B). The bilateral arms 220A-B or palatal region 210 can terminate in connecting portions (referred to herein as a first tooth engagement region 222A and a second tooth engagement region 222B) that connect to one or more teeth of the patient (e.g., to one or more of the molars and/or premolars of the patient). In some embodiments, the palatal region 210 can press against the roof, or palate, of the oral cavity. Alternatively, the palatal region 210 may not contact the palate or roof of the patient's mouth.

First and second tooth engagement regions 222A-B can be at the terminus of bilateral arms 220A-B or palatal region 210. In some embodiments, first and second tooth engagement regions 222A-B each include a lingual portion, an occlusal portion, and/or a buccal portion. In some embodiments, first and second tooth engagement regions 222A-B include a lingual portion that engages with lingual portions of one or more teeth (e.g., without including a buccal portion or occlusal portion). First and second tooth engagement regions 222A-B can be form fitted to a patient's teeth (e.g., molars and/or premolars) in embodiments. Alternatively, first and second tooth engagement regions 222A-B may be formed to engage with the lingual region of an orthodontic aligner to be worn by the patient. When the palatal expander 200 is inserted onto a patient's upper dental arch, the first and second engagement regions 222A-B can securely attach the palatal expander 2000 to the patient's molars or premolars (or to one or more portions of an orthodontic aligner).

In embodiments, retention attachments may be bonded to a patient's teeth, and palatal expander may include one or more receiving wells 224 that are shaped to engage with the retention attachments to secure the palatal expander to the patient's palate. In embodiments, the palatal region of the palatal expander is configured to apply a lateral force or transverse force across a palate of a patient when the first tooth engagement region is secured to one or more first teeth and the second tooth engagement region is secured to one or more second teeth of the patient. The lateral force may cause the patient's palate to expand over time.

In embodiments, palatal expander 200 can include one or more integrated sensors 236. The one or more integrated sensors 236 may be located at various regions of the palatal expander, and may include force sensors, pressure sensors, temperature sensors, contact sensors, capacitive sensors, displacement sensors, colorimetric sensors, biofilm sensors, touch sensors, motion sensors (e.g., accelerometers, rotational sensors, gyroscopes, etc.), and/or other types of sensors. In some examples, bilateral arms 220A-B can include one or more sensor(s) 236. In some examples, palatal region 210 may include one or more sensors 236. Sensor(s) 236 can be integrated into the material of the palatal expander 200A-B and/or disposed at a surface of palatal expander 200A-B.

In embodiments, tooth engagement regions 222A-B can each include one or more integrated sensor(s) 236. In alternate or additional embodiments, sensor(s) 236 can additionally or alternatively be placed or integrated within any other portion or region of palatal expander 200 (e.g., at portions 220A-B, at palatal region 210, etc.). In some cases, any number of sensors can be integrated into any combination of the above portions, as is feasible or appropriate.

In embodiments, sensors 236 can be or include a force sensor. A force sensor can be capable of detecting and quantifying the magnitude of forces applied to or by the palatal expander 200 to or by dentition (e.g., molars or premolars) of the patient. Such a sensor can be or include a strain gauge, a piezoelectric sensor, a capacitive sensor, an optical force sensor, a magnetic force sensor, a strain gauge transducer, a flexure sensor, and so on. In embodiments, one or more force sensors as configured and positioned to measure a transverse or lateral force on the palatal expander 200A-B. For example, some force sensors may be disposed in tooth engagement regions 222A-B to measure bite force.

In embodiments, sensors 236C can be or include one or more pressure sensors. Such a sensor can be capable of detecting and quantifying the pressure applied to or by the palatal expander 200 to dentition (e.g., molars or premolars) of the patient. Such a sensor can be or include a piezoresistive pressure sensor, an optical pressure sensor, a capacitive pressure sensor, a strain gauge pressure sensor, a piezoresistive pressure sensor, a resonant pressure sensor, a thermal pressure sensor, a potentiometric pressure sensor, and so on. In some examples, the sensor may be a deformable pressure sensor module that encapsulates circuitry for measuring a pressure within a fluid-filled cavity surrounded by a deformable membrane, wherein the pressure within the cavity is based on an intraoral pressure that deforms the membrane. More information about a deformable pressure sensor module can be found in U.S. patent application Ser. No. 18/624,107, which is incorporated by reference herein in its entirety.

In embodiments, sensors 236 can be or include one or more displacement sensors. Displacement sensors can be capable of detecting and quantifying the amount of movement or displacement of teeth, an upper palate of a patient, etc. from the palatal expander or of parts of the palatal expander 200 from other parts of the palatal expander. Such a sensor can be or include an inductive displacement sensor, an ultrasonic displacement sensor, and optical displacement sensor, a laser sensor, a potentiometric displacement sensor, a linear variable differential transformer (LVDT), a magnetoresistive displacement sensor, a hall effect sensor, a piezoelectric displacement sensor, and so on.

In embodiments, sensors 236 can be or include one or more colorimetric sensors. Such a sensor can be capable of detecting and quantifying changes in color of the palatal expander 200, or the surrounding tissue and environment of the oral cavity. Such a sensor can be or include an image sensor, or any other similar sensor. In some embodiments one or more colorimetric sensors change colors responsive to conditions of the palatal expander, such as a pH, a temperature, an amount of force or pressure applied by the palatal expander (which may vary, e.g., as the palatal expander experiences stress relaxation, due to manufacturing defects, etc.), an amount of force or pressure exerted against the palatal expander (e.g., when the palatal expander experiences compression as it is worn by a patient), and so on. Such a color change may be captured by a patient taking an image of the palatal expander, and through color analysis of the image in some embodiments.

In embodiments, sensor(s) 236 can be or include one or more chemical or biological sensors. For example, the sensor may be a biofilm sensor capable of detecting and quantifying the growth of biofilm on the palatal expander 200, and/or on the surrounding tissue and environment of the oral cavity (e.g., teeth, gingiva, palate, tongue). Such a sensor can be or include an electrochemical sensor, a microbial fuel cell (MFC), an impedance-based sensor, an optical sensor, an acoustic sensor, a microbial biosensor, a microfluidic sensor, or any other similar sensor.

In embodiments, sensor(s) 236 can be or include one or more temperature sensors. Such a sensor can be capable of detecting and quantifying the temperature of the palatal expander 200, and/or the surrounding tissue and environment of the oral cavity. Such a sensor can be or include a thermocouple sensor, a resistance temperature detector (RTD), an infrared sensor, an optical sensor, and so on.

In embodiments, sensor(s) 236 can be or include one or more sensors capable of sensing rotational forces/pressures (e.g., torque or a rotational moment). The sensor(s) 236 may include, for example, torque sensors. In some embodiments, sensor(s) 236 are capable of measuring linear force/pressure along 3 axes as well as rotational force about three axis. Accordingly, in embodiments the sensor(s) 236 are force sensors that measure forces along 6 degrees of freedom. In some embodiments, one or more such sensors 236 are located at palatal region 210 of palatal expander 200A-B, and measure rotational forces between a first side of palatal expander 200A-B and a second side of palatal expander 200A-B. For example, a sensor 236 such as a torque sensor may measure a rotational force between a first palatal region of the palatal expander 200A-B and a second palatal region of the palatal expander 200A-B. Such sensor(s) 236 may be used to detect, for example, an amount of rotation of a left side of a patient's palate relative to a right side of the patient's palate in embodiments. In one embodiment, one or more force sensors or pressure sensors capable of measuring rotational force 236 (or rotational pressure) are located at tooth engagement regions 222A-B (also referred to simply as tooth regions). Such sensor(s) 236 may detect rotational force on one or more teeth (e.g., molars), and may be used to detect tipping of teeth in embodiments.

In embodiments, sensor(s) 236 can be or include one or more force sensors or pressure sensors at tooth engagement regions 222A-B that measure a force and/or pressure associated with patient bite (e.g., a bite force, which may be a vertical force or a force that is normal to a surface of the tooth engagement regions 222A-B in some embodiments). Such sensor(s) 236 may be used to count a number of bites experienced by palatal expander 200A, measure a bite force experienced by palatal expander 200A, and so on. In some embodiments, multiple force sensors 236 or pressure sensors are positioned at different tooth positions of both tooth engagement region 222A and tooth engagement region 222B, and the bite forces measured by the multiple sensors 236 may be used to perform a bite analysis of a patient and to determine bite contacts. Such information may be compared against ideal bite contacts for the patient. If detected bite contacts deviate from ideal bite contacts, then one or more new palatal expanders may be manufactured that have changed thicknesses in one or more portions of tooth engagement regions 222A-B to correct the bite contacts of the patient. A report of the bite analysis (e.g., indicating the bite contacts, comparison to ideal bite contacts, recommendations to correct bite contacts, etc.) may be output to a display of an output device (e.g., a client device of a doctor and/or patient).

Any one or more of the aforementioned sensors may also be included in other dental appliances such as an orthodontic aligner in embodiments, and may be used to measure similar properties to those discussed above and below.

In embodiments, the palatal expander 200A-B can be constructed of one or more materials that are biocompatible and capable of exerting a lateral or transverse force to expand the patient's palate. For instance, in embodiments, the palatal expander 200A-B can be made from metals (e.g., stainless steel, nickel-titanium alloys, etc.), or medical medical-grade plastics (e.g., medical-grade acrylics, thermoplastics, etc.). In embodiments, palatal expander 200A-B can be made from any material that is capable of being 3D printed (e.g., biocompatible resins, thermoplastics, etc.). In embodiments, palatal expander 200 and its specific portions can be any combination of the above or similar materials. For instance, in embodiments, palatal region 210 can be made from a metal (e.g., when designed to include a clearance so as to not contact the palate or other patient tissue), while connection portions 222A-B can be made of a softer material (e.g., a thermoplastic) due their direct contact with patient tissue (e.g., teeth, gingiva). In another example, palatal expander 200A-B can include an inner portion formed of a material with high structural rigidity and a softer outer portion formed of a second material with a lower structural rigidity. In embodiments, the inner portion and the outer portion are each polymer-based materials. In embodiments, the inner portion and the outer portion are 3D printed.

In embodiments, the palatal expander corresponds to a palatal expander of U.S. Pat. No. 11,576,750, issued Feb. 14, 2023, which is incorporated by reference herein in its entirety. In further embodiments, the palatal expander corresponds to a palatal expender of U.S. Patent Publication No. 2023/0172692, published Jun. 8, 2023, which is incorporated by reference herein in its entirety.

In embodiments, the bilateral arms 220A-B and/or palatal region 210 can exert a transverse force onto the molars and/or premolars of a patient. The bilateral arms 220A-B and/or palatal region 210 can ensure a balanced distribution of force across the palate in the transverse direction (e.g., across the palate of the patient, as seen by functional width 260). Functional width 260 can be the distance between first and second tooth engagement regions 222A-B in some embodiments (e.g., the distance between the left and right molars of the patient). This can correspond to an arch width of the patient.

In embodiments, the functional width 260 can be adjustable (as previously discussed with respect to FIG. 1). In cases, the palatal expander 200A-B can be a fixed-length palatal expander. In either case, one or more of such or similar palatal expanders can be used in a series of palatal expanders and corresponding stages to allows for a stepwise expansion (and/or retention) of the palate.

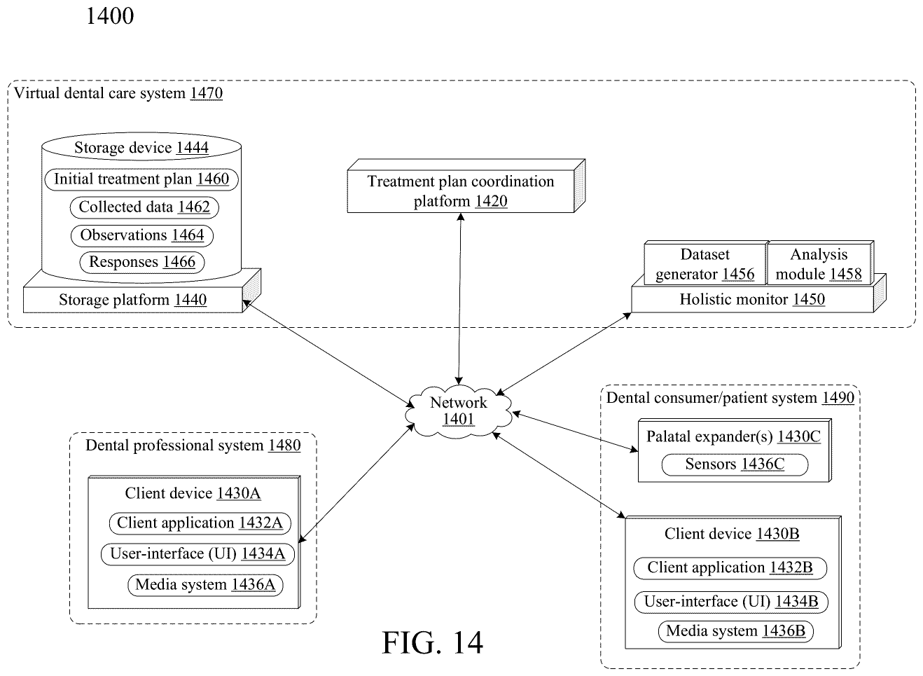

In embodiments, the palatal expander 200A-B can wirelessly transmit data collected via the integrated sensors 236 as it is being used within a palatal expansion treatment plan. In embodiments, the integrated sensor(s) 236 can be wirelessly connected to a device of the user (e.g., a phone). In embodiments, the palatal expander 200 can wirelessly connect to a storage container for the palatal expander 200A-B. In embodiments, the palatal expander 200A-B includes a wireless communication module, and the wireless communication module wirelessly connects to an external device (e.g., mobile device, storage container, stationary device, etc.) via a first wireless communication protocol, such as Bluetooth, Zigbee, Wi-Fi, and so on. The device or storage container may connect to a local network (LAN) and/or a wide area network (WAN) via a second wireless communication protocol such as Wi-Fi. Alternatively, the device or storage container may connect to a cellular network via a 3G, 4G, 5G, etc. communications protocol. In either case, the device or storage container may provide captured sensor data to a server that may process the sensor data to make determinations about the palatal expander and/or palatal expansion progress and/or a patient's oral health in embodiments. Alternatively, the palatal expander 200A-B may directly connect to a network (e.g., LAN, WAN, cellular network, etc.) and may send sensor data to a server via the network without first sending the sensor data to a local device (e.g., a mobile device or storage container that then forwards the data on). In embodiments, the sensor data is ultimately provided to a platform (e.g., platform 1420 as described with respect to FIG. 14) associated with a palatal expansion treatment plan.

Returning now to FIG. 1, in some cases, the stages 100A-D can involve progressing from an expansion stage to a retention stage. For instance, in embodiments, stages 100A-100C can be expansion stages, designed to expand the palate from a first width to successively larger widths. As mentioned, during such stage(s), a palatal expander can be adjusted and/or interchanged (e.g., such that functional width 260 of FIG. 2A is increased) at regular intervals to apply transverse or lateral forces to the upper molars and/or premolars. Once a target amount of palatal expansion is achieved, a retention stage 100D, may not expand the palate further, but may retain the palate at the current width (e.g., functional width 260 can cease to be increased, and remain static). For instance, after achieving a target level of expansion in stages 100A-C, stage 100D can be a retention stage. During this stage 100D, the functional width 260 of the palatal expander 200 can be identical to the functional width of the previous expansion stage 100C. This stage can allow new bone to form and solidify along an expansion suture. This stage can allow stabilization of the new width of the palate. In some embodiments, one or more retention stages are provided between expansion stages in addition to at the completion of expansion.

In embodiments, the width of the palate can be measured by an arch width of the patient, or the functional width of the palatal expander (e.g., functional width 260 of FIG. 2A). In embodiments, each expansion stage can be characterized by an expansion rate, which may be a change in arch width (and/or a change in functional width 260) over time (e.g., over the period in which an expansion stage lasts).

During palatal expansion, a progress of a palatal expansion treatment plan may be virtually monitored in accordance with embodiments of the present disclosure. The progress of palatal expansion may be monitored based on assessment of sensor data gathered by integrated sensors of one or more palatal expanders and/or based on image data captured by a patient. The image data may be of the patient's upper dental arch and/or of the palatal expander(s). In some embodiments, one or more images of a patient's face and/or smile may also be captured and used to perform an assessment associated with one or more stages of palatal expansion treatment and/or orthodontic treatment. In some embodiments, the patient is directed to take one or more images of their upper dental arch and/or face at set time periods. These images may be uploaded to a server that may process the images to make one or more determinations, as described herein below. Images may be processed using traditional image processing techniques and/or the application of machine learning in embodiments. Sensor data may additionally or alternatively be uploaded to the server, which may or may not use trained machine learning models to process the sensor data.

FIG. 3 illustrates an example monitoring process 300 that can be used during implementation of the treatment plan of FIG. 1, according to some embodiments of the present disclosure. The monitoring process 300 can include operations such as data collection 3.1, data processing 3.2 and response generation 3.3. Some operations (e.g., data collection 3.1) may be at least partially performed by palatal expanders, devices that include one or more cameras, and so on. Some operations (e.g., data collection 3.1, data processing 3.2, response generation 3.3) may be performed by a computing device such as a client device, a server computer device, and so on, which may receive and process data. One or more operations of monitoring process 300 (e.g., data collection 3.1, data processing 3.2 and/or response generation 3.3) may be performed by a processing logic, which may include software, firmware, or a combination thereof that executes on one or more processors.

Components, processes, and features as seen and described with respect to FIG. 3 can correspond, or be similar, to similar components as seen and described with respect to FIGS. 1-2B. Thus, embodiments discussed with respect to FIG. 3, can incorporate and augment at least the embodiments described with respect to FIGS. 1-2B.

At operation 3.1, the monitoring process 300 can begin with data collection. The system can collect data 362 through a sensor device and/or sensors. As discussed with respect to FIGS. 1-2B, the sensor device used to collect data 362 can be a palatal expander (e.g., such as palatal expander 200A-B of FIGS. 2A-B), an image sensor, or a separate imaging sensor (e.g., of a camera, mobile device, etc. external to the palatal expander) or 3D scanner (e.g., intraoral scanner) capable of capturing or generating image data including images of the oral cavity and/or palatal region of a patient.

Sensors used to collect data 362 can be or include a sensor integrated into a palatal expander. Sensors can be or include any of the sensors discussed with respect to FIGS. 2A-B, and incorporate and augment at least the embodiments described therein. Accordingly, data 362 can be or include force measurements, pressure measurements, displacement measurements, colorimetric measurements (e.g., measurements indicating a level of staining of the device or palatal expander), biofilm measurements (e.g., measurements indicating a level of biofilm build-up), movement measurements, acceleration measurements, rotation measurements, and/or temperature measurements, to name a few.

Sensors that collect data 362 can further be or include an image sensor (e.g., a camera) of a device external to the palatal expander, and data 362 can be or include image data of the patient's upper palate, dental arch, face, smile, etc. In embodiments, sensors that collect data 362 can belong to a personal client device (e.g., a phone, tablet computer, digital camera, etc.), for example, of the patient or doctor. In embodiments, the patient can themselves capture image data (e.g., self-take an image) of their oral cavity, of their face, etc. Accordingly, data 362 can be image data that can be or include images taken of the occlusal surfaces of the upper dental arch.

As discussed, such data 362 can be collected via transmission through a client device, or direct transfer, to a platform associated with the system and on to the holistic monitor (e.g., holistic monitor 1450 as described with respect to FIG. 14).

At operations 3.2 and 3.3, the process 300 can include performing data processing 3.2 on the collected data 362 to form observations 364. In embodiments, processing 3.2 can be performed via a holistic monitor (e.g., monitor 1450 of FIG. 14). Data processing 3.2 may be performed using application of machine learning and/or using traditional image processing techniques in embodiments. Following operation 3.2, at operation 3.3, the process 300 can include generating and/or executing responses 366 based on the received observations 364. In embodiments, responses 366 can also be formed via a virtual dental care system (e.g., virtual dental care system 1470 of FIG. 14).

In embodiments, data processing 3.2 can be further discretized into progress monitoring 3.2A, event monitoring 3.2B, and retention monitoring 3.2C. Such discretization can correspond to similar discretization of observations 364. For instance, in embodiments, progress monitoring 3.2A can include monitoring metrics and/or measurements associated with a progression of palatal expansion (e.g., arch-width expansion, force or pressure during an expansion stage, etc.). In embodiments, the observations 364 may include features and/or measurements characterizing a rate of palatal expansion. In embodiments, observations 364 can be or include progress indicators, indicating the rate of progress of a patient along a palatal expansion treatment plan. The event monitoring 3.2B portion of data processing 3.2 can include monitoring for the occurrence of events within the associated palatal expansion treatment plan (e.g., the eruption of teeth, the formation of a diastema as the palatal expansion treatment plan is progressing, and so on). The retention monitoring 3.2C portion of data processing 3.2 can include monitoring for metrics and data associated with a retention stage of an associated palatal expansion treatment plan. Accordingly, observations 364 can correspond to data 362 gathered during one or more stages (e.g., expansion stages and/or retention stages) of a palatal expansion treatment plan.

From a generalized viewpoint, such observations 364 can serve as tools to characterize a treatment plan. Observations 364 may be used to determine if movement or expansion of the palate is occurring as planned, if movement of teeth is happening, if certain events are occurring, a level of progress of the patient with respect to the palatal expansion treatment plan, and/or the rate of palatal expansion, whether a patient's facial appearance is changing in an acceptable manner, and so on. In embodiments, observations 364 can be formed by comparing collected data 362 against stored or historical data (e.g., previously collected data) taken from a prior checkpoint, by application of image processing algorithms, by application of machine learning or artificial intelligence, and so on. In some embodiments, observations 364 can be determined by comparing one or more of the collected data to projected or predicted targets and goals. For example, in some embodiments, an observation (within observations 364) can be or include the current width of the patient's dental arch. Such a condition and measurement can be within image data (e.g., included within collected data 362) that was generated based on an intraoral scan or 2D image(s) of the patient's dental arch. In embodiments, a comparison can be made between a patient's actual arch width and a target arch width. For example, in some cases, an arch width may be computed from an image of the patient's upper dental arch, and the arch width may be compared to an arch width measured from a 3D model of the patient's dental arch for a current stage of treatment in the treatment plan. In such a case, the comparison can produce deviations, or characterize a level of progression, for the current treatment stage and the multi-stage orthodontic treatment plan at large.

In some embodiments, a measured transverse force or pressure on one or more palatal expanders worn by the patient may be compared to one or more force or pressure thresholds. Decisions to extend the time of one or more treatment stages and/or reduce the time of one or more treatment stages may be made based on a result of the comparison.

Based on comparison of currently collected data, previously collected data, and/or a palatal expansion treatment plan, observations 364 can be made for a given treatment stage. Observations may include one or more metrics characterizing a level of expansion achieved by one or more palatal expanders. Observations may include, for example, observations that a patient's palate is expanding slower than anticipated, that a force buildup (e.g., increase in measured force over time between expander treatment stages) on a patient's dental arch is tooth high, that the patient's palate is expanding faster than anticipated, that a force applied to a patient's dental arch by a palatal expander is too low, and so on. Such observations may include observations made based on analysis of sensor data generated by integrated sensors of the palatal expander and/or based on images of the patient's upper dental arch and/or of the palatal expander(s). Observations may also include one or more identified events, such as formation of a diastema, tilting of one or more patient teeth, formation of lesions or lacerations, contact or force between the palatal expander and the upper palate of the patient, and so on (other examples described below). Observations may additionally or alternatively include a measured bite force, an identification of bruxism, a count of a number of bites experienced by a palatal expander or orthodontic aligner, determined bite contact points, a determination of how much time a patient wears a palatal expander or orthodontic aligner, a bite analysis, and so on. Observations made based on facial images may additionally or alternatively include predictions of a post-treatment facial width, a post-treatment nose breadth, a post-treatment facial asymmetry, and so on.

Response generation 3.3 can then be performed to determine any modifications for the palatal expansion treatment plan for achieving the treatment goals, which may be made based on a determined level of progress. For example, if an observation (within observations 364) indicates that the patient treatment is behind schedule or that a force on a patient's upper dental arch is too high, the current stage of treatment can be prolonged. If such a comparison indicates the patient treatment is advancing more rapidly than expected, or a force on the patient's upper dental arch is too low, in some cases the patient can be advanced to the subsequent treatment stage earlier than planned. In such a way, observations 364 based on collected patient data can be used to determine a patient's level of progression with respect to a stage of a palatal expansion treatment plan, and the treatment plan at large. Additionally, observations may be used to determine whether a patient's post-treatment facial appearance is acceptable to a patient. For example, a treatment plan may be altered to speed up treatment (e.g., by reducing the amount of time the patient is required to wear a palatal expander of an upcoming stage or a set of palatal expanders of a set of upcoming stages). As another example, a treatment plan may be altered to slow down treatment (e.g., by increasing the amount of time the patient is required to wear a palatal expander of an upcoming stage or a set of palatal expanders of a set of upcoming stages). These alterations may occur dynamically and may occur continually based on continued measurements as a patient's treatment progresses. It may be difficult to predict at the beginning of treatment how a patient responds to treatment; so continued monitoring and alteration of the treatment plan as disclosed herein allows for optimizing the patient's treatment for the duration of the treatment to allow for fast, safe, and effective treatment.

After generation of observations 364, responses 366 can be formed and/or executed in response to a particular observation (within observations 364). For example, should observations 364 include a specific observation that a measured force is surpassing a maximum threshold, a response (within responses 366) can be formed that is or includes an update to the palatal expansion treatment plan. Such an update can include extending the current stage of the treatment plan. In other cases a response can include an update to the palatal expansion treatment plan that can be or include advancing a patient to the subsequent stage. In embodiments, such can be indicated to a patient on a device associated with the patient, and/or corresponding HCP via a notification (which can be a response of responses 366 that is independent of the update to the palatal expansion treatment plan). For example, a notification may be pushed on a client device (e.g., a smartphone, a tablet, a PC) associated with the patient or HCP, and/or may be sent as a communication (e.g., an email, a text message). This notification may direct the patient or HCP to advance to the next stage by removing a current palatal expander and replacing it with the next palatal expander in a planned sequence.

Notifications may also be provided about identified risks of adverse effects. For example, based on the data processing 3.2, processing logic may identify one or more possible adverse effects associated with palatal expansion treatment that have occurred or that have an increased likelihood of occurring based on the collected data. For example, if a sensor on the palatal expander detects contact with the palate or a force against the palate that is above a threshold, processing logic may determine that the patient is likely to develop a lesion or irritation where the palatal expander is contacting the palate, and may output a warning. In another example, a notification may be provided indicating that a patient's predicted facial width, nose breadth, facial symmetry, etc. is deviating from acceptable levels.

Notifications may also indicate whether or not a treatment is proceeding as planned (e.g., that a treatment is not on track). In an example, such a notification may be based on a detected level of force being below a threshold. Such a low force may indicate that the palatal expander is not applying sufficient force to expand the patient's palate, for example.

Processing logic may provide suggestions for a patient, such as to floss or brush to prevent cavities, to clean the palatal expander to address biofilm buildup, to wear the palatal expander longer (e.g., to be compliant with suggested wear times for the palatal expander), and so on. A suggestion to clean the palatal expander may be output responsive to detecting that a biofilm level is above a biofilm level threshold, for example. A suggestion to wear the palatal expander longer may be output responsive to determining that the patient is not wearing the palatal expander for a recommended amount of time. Processing logic may determine when the palatal expander is being worn, for example, based on detecting forces on the palatal expander. When at least a threshold amount of force is not detected by one or more sensors of the palatal expander, processing logic may determine that the palatal expander is not being worn. If the palatal expander is not worn for a recommended amount of time (e.g., patient fails to comply with a designated amount of wear time for a palatal expander), a notification for the patient to wear the palatal expander longer may be generated.

In some embodiments, observations 364 and/or collected data are processed using one or more trained machine learning model to generate one or more responses 366. For example, the trained machine learning model may be trained to receive an input of image data, sensor data and/or observations about image data and/or sensor data, and to output recommendations (e.g., such as for changes to a treatment plan), notifications and/or predictions.

Given the rapidity and non-invasiveness associated with remote data collection, in embodiments, process 300 can be run at any routine, or predefined schedule. This can include any feasible level of granularity (e.g., hourly, daily, weekly, etc., as previously discussed) with respect to the overall treatment strategy. Thus, process 300 can be employed to continuously provide updates to further personalize and optimize the treatment plan for a specific patient. In some embodiments, the frequency with which to gather updated patient data and assess a patient's treatment progress based on the patient data can be updated by process 300. For example, if the patient is responding to treatment faster than anticipated and will be advanced to a subsequent treatment stage earlier than anticipated, a response (within responses 366) can include a recommendation for more frequent collection of patient data. For instance, an initial plan can include an initial checkpoint schedule, that may or may not have been personalized for the patient based on patient data (e.g., such as patient demographics including age, gender, medical history, and other relevant characteristics). In embodiments, these checkpoint schedules can be modified and updated dynamically based on the patient's response to the treatment, adherence to prescribed routines, and other relevant indicators of progress (as indicated within observations 364 and/or responses 366).

Thus, the process 300 can allow patients to accelerate, or skip stages, to prolong stages, etc. based on analysis of the collected data 362, and further personalize a treatment plan. Thus, the length of each stage of a treatment plan can be optimized, or personalized, with further precision and granularity. In embodiments, this can save a patient, and an HCP, valuable time and resources, and overall increase the effectiveness of a treatment plan.

In some embodiments, the rationale and/or process for arriving at a plan update and/or level of progression (e.g., within responses 366) can be provided to the patient, as a method of incentivization for the patient to increase or maintain compliance. In an example, in cases where a response (of responses 366) is to retain the patient at the current stage, either due to non-compliance or some other factor, such underlying factors can be presented to the patient. Such a presentation can function to link patient behaviors, biology, genetics, etc., to the plan updates or outcomes. For example, in a case where a patient's consumption of certain food or materials is negatively impacting the progress of the palatal expansion treatment plan, data indicating such can be presented to the patient. This can “link” such behavior to outcome, and further incentivize the patient to enhance their rate of compliance. Similarly, if certain efforts or actions of the patient are resulting in accelerated or quickened treatment, such a positive link can be presented to the patient so as to further incentivize such positive behaviors. Thus, through such transparency, process 300 and system at large can incorporate an additional factor for increasing efficiency of the treatment plan.

In embodiments, data processing 3.2 can be performed to determine a level of expansion force at various stages of a palatal expansion treatment plan. Forces between stages may be compared in embodiments to determine force changes (e.g., force buildup in the instance that forces are increasing with subsequent stages of treatment). Force build-up between stages is the increase in force that is detected between palatal expansion stages. For example, if a first palatal expander measured a first force and a second palatal expander measured a second higher force, then delta between the second and first forces may constitute a force buildup between stages. In embodiments, a force sensor can measure expansion force from molars or premolars exerted onto the sensor device. Data processing 3.2 can be performed to analyze historical, or time-series, values of such force measurement data to determine a force profile based on the transverse force (or pressure) measured by one or more palatal expanders in a series of palatal expanders. Data processing 3.2 may be performed to determine a change in forces measured by the force sensor(s) of one or more palatal expanders over time. The force profile may be compared to one or more force profile criteria (such as force profile criteria for advancing to a next treatment stage). If the force profile satisfies the one or more force profile criteria (e.g., an expansion force criterion and/or force buildup (e.g., force change) criterion), processing logic may determine to take one or more actions (such as advancement to a next treatment stage). Should the expansion force from one or more treatment stages reach a sufficient level or threshold (e.g., surpass a maximum allowable level of force), an observation of such can be formed (within observations 364).

In cases where observations 364 include an observation that the expansion force has surpassed a maximum allowable threshold, operation 3.3 can generate a response (within responses 366) that includes an update to the current palatal expansion treatment plan. In embodiments, the update can be that the patient should be held at the current treatment stage and/or current palatal expander, or that the patient should revert back to a previous treatment stage and hold it there (e.g., until the patient is determined ready to proceed, until a new palatal expander with an acceptable expansion force can be manufactured). This holding, or pausing, can continue, until an observation indicates that the force is once again within an acceptable range (e.g., that the force drops). Should the expansion force surpass a threshold multiple times, observations 364 can include an indication of such. In response, response generation 3.3 can be performed to alter a rate of palatal expansion for the patient. Thus, data processing 3.2 and response generation 3.3 can be used to respond to multiple events, or occurrences, appropriately. Thus, updates to the palatal expansion treatment plan produced at response generation 3.3 can be performed to alter any number of stages of the palatal expansion treatment plan.

Data processing 3.2 can be used to further determine a level of force reduction between stages (e.g., between sequential retention stages) of a palatal expansion treatment plan. For instance, in embodiments, a force sensor can measure forces over time, from molars or premolars exerted onto the sensor device of one or more retention devices. A force profile may be determined based on measurements taken over a time period. Processing logic may determine whether the force profile satisfies one or more force profile criteria. The criteria may include, for example, a criterion that the force is decreasing over time (e.g., due to stress relaxation) and/or a criterion that the force is below a force threshold. Data processing 3.2 can thus be used to analyze historical, or time-series, values of such force measurement data. Should the force reduce below a threshold (e.g., fall below a minimum allowable level of force), for example, data processing 3.2 can be performed to indicate such for processing via response generation 3.3 operations as an observation (within observations 364).

In cases where observations 364 include an observation that the force reduction has fallen below a minimum threshold during a retention stage, response generation 3.3 can be performed to generate a response (within responses 366) that includes an update to the current palatal expansion treatment plan. The update can be that the patient has completed a retention stage and/or that the patient should be advanced to the next stage of the treatment (e.g., that one or more orthodontic alignment stages of treatment should begin). A subsequent treatment stage can include application of a stage 1 to 2 retainer, application of orthodontic aligners, or other treatment.

Data processing 3.2 can be performed to determine a level of insertion and/or removal force required for inserting and/or removing a palatal expander from a patient's oral cavity. For instance, in embodiments, a force sensor can be placed within the buccal region of the palatal expander. The force sensor can measure insertion and/or removal force exerted onto the palatal expander as the palatal expander is being inserted or removed. Data processing 3.2 can be performed to analyze force measurement data gathered during insertion and/or removal. Should the insertion and/or removal force reach a sufficient level or threshold (e.g., surpass a maximum allowable level of force), data processing 3.2 can be performed to indicate such as an observation (within observations 364).

In cases where observations 364 include an observation that the insertion and/or removal force has surpassed a maximum allowable threshold, response generation 3.3 can be performed to generate a response (within responses 366) that includes sending a notification to the patient. The notification can include instructions for proper insertion and removal of the palatal expander.

Data processing 3.2 can be performed to determine a level of fit for a palatal expander (e.g., whether a palatal expander is properly seated) into a patient's palatal region and/or oral cavity. For instance, in embodiments, a displacement sensor can be positioned within the occlusal region of the palatal expander, at one or more tooth engagement portions of the palatal expander, etc. The displacement sensor(s) can measure a distance between the palatal expander and the patient's upper palate and/or contact between the palatal expander and the patient's upper palate. Displacement sensor(s) and/or contact sensors may also measure a distance between the palatal expander and one or more teeth (e.g., molar and/or premolar) of the patient and/or contact between the palatal expander and the patient's teeth.

Data processing 3.2 can be performed to analyze distance and/or contact measurement data gathered by the sensor device. Should the displacement measurement data for distance between the palatal expander and the patient's teeth lie outside of a pre-determined threshold range (e.g., surpass a minimum or a maximum allowable level of displacement), data processing 3.2 can indicate such as an observation (within observations 364). Similarly, where the distance and/or contact measurement data indicates a distance between the palatal expander and the upper palate of the patient is below a threshold, an observation may be generated.

In cases where observations 364 includes an observation that the level of displacement has surpassed a minimum or a maximum allowable level of displacement with respect to a distance between the palatal expander and the patient's teeth, response generation 3.3 can be used to generate a response (within responses 366) that includes delivering a notification to a patient. The notification can include an indication of the level of fit. The notification can include instructed to use chewies to fully seat the palatal expander on the dentition. In embodiments, the notification can be provided to a client device of the patient, and can be displayed on the client device of the patient.

In cases where observations 364 includes an observation that the level of displacement is below a minimum allowable level of displacement with respect to a distance between the palatal expander and the upper palate (e.g., palatal expander is impinging on the upper palate), response generation 3.3 can be used to generate a response (within responses 366) that includes a suggestion to replace the palatal expander with a new palatal expander having an altered shape that will not impinge on the patient's upper palate. This may be done to reduce damage to the patient's upper palate and/or patient discomfort.

Data processing 3.2 can be used to determine a level of staining to palatal expander. For instance, in embodiments, a colorimetric sensor can measure a level, or change in staining on the palatal expander. Data processing 3.2 can be used to analyze colorimetric or staining measurement data. Should the colorimetric or staining measurement data lie outside of a pre-determined threshold range (e.g., surpass a maximum allowable level of staining), data processing 3.2 can indicate such as an observation (within observations 364).

In cases where observations 364 include an observation that the level of staining has surpassed a maximum allowable threshold, response generation 3.3 can generate a response within responses 366 that includes delivering a notification to a patient. The notification can include an indication of the level of staining. The notification can include a message that the patient should avoid certain high-staining materials (e.g., such as risky foods or drinks).

Data processing 3.2 can be performed to determine a level of biofilm build-up on the palatal expander. For instance, in embodiments, a biofilm sensor (or other chemical or biological sensor) can measure a level, or change in biofilm on the palatal expander. Data processing 3.2 can be used to analyze such biofilm measurement data. Should the biofilm measurement data lie outside of a pre-determined threshold range (e.g., surpass a maximum allowable level of biofilm), data processing 3.2 can indicate such as an observation (within observations 364).

In cases where observations 364 includes an observation that the level of biofilm has surpassed a maximum allowable threshold, response generation 3.3 can generate a response within responses 366 that includes delivering a notification to a patient. The notification can include an indication of the level of biofilm build-up. The notification can include a message that the patient should avoid certain materials that induce biofilm buildup (e.g., such as risk foods or drinks).

Data processing 3.2 can be used to determine if a temperature level within a region of the a palatal expander is acceptable. For instance, in embodiments, a temperature sensor can measure a level, or change, in the temperature of the patient's palatal region, the palatal expander, and/or oral cavity of the patient while it is inserted. Data processing 3.2 can be used to analyze such temperature measurement data. Should the temperature measurement data lie outside of a pre-determined threshold range (e.g., a maximum allowable temperature), data processing 3.2 can be performed to indicate such as an observation (within observations 364).

In cases where observations 364 include an observation that the temperature has surpassed a maximum allowable threshold, response generation 3.3 can generate a response within responses 366 that includes delivering a notification to a patient. The notification can include an indication of the temperature. The notification can include a message that the patient is consuming excessively hot food or drink which can negatively affect the function of the palatal expander.

Data processing 3.2 can be used to determine if an amount of rotational force between a first palatal region (e.g., left side) and a second palatal region (e.g., right side) of a palatal expander is within an acceptable range. For example, rotational force may be measured and compared to planned rotational forces in a similar manner as discussed above and below with reference to transverse or lateral forces. Such force assessments may include assessment of force buildup between or across multiple palatal expansion treatment stages, assessment of force reduction between treatment stages, and so on. In some embodiments, it may be determined that an amount of rotational force deviates from a planned rotational force by more than a threshold amount. In response to this determination, a treatment plan may be altered in embodiments. For example, a treatment plan may be altered to speed up treatment (e.g., by reducing the amount of time the patient is required to wear a palatal expander of an upcoming stage or a set of palatal expanders of a set of upcoming stages). As another example, a treatment plan may be altered to slow down treatment (e.g., by increasing the amount of time the patient is required to wear a palatal expander of an upcoming stage or a set of palatal expanders of a set of upcoming stages). These alterations may occur dynamically and may occur continually based on continued measurements as a patient's treatment progresses. It may be difficult to predict at the beginning of treatment how a patient responds to treatment; so continued monitoring and alteration of the treatment plan as disclosed herein allows for optimizing the patient's treatment for the duration of the treatment to allow for fast, safe, and effective treatment.

In cases where observations 364 include an observation that the rotational force has surpassed a maximum allowable threshold or that is below a minimum allowable threshold, operation 3.3 can generate a response (within responses 366) that includes an update to the current palatal expansion treatment plan. In embodiments, the update can be that the patient should be held at the current treatment stage and/or current palatal expander, or that the patient should revert back to a previous treatment stage and hold it there (e.g., until the patient is determined ready to proceed, until a new palatal expander with an acceptable expansion force can be manufactured), for example. This holding, or pausing, can continue, until an observation indicates that the force is once again within an acceptable range (e.g., that the force drops). Should the rotational force surpass a threshold multiple times, observations 364 can include an indication of such. In response, response generation 3.3 can be performed to alter a rate of palatal expansion for the patient.

Data processing 3.2 can be used to determine if a rotational force on one or more teeth (e.g., molars) is within an acceptable range. For example, rotational force exerted on one or more teeth may be measured and compared to one or more rotational force (i.e., torque) thresholds.

In cases where observations 364 include an observation that the rotational force on one or more teeth has surpassed a maximum allowable threshold, operation 3.3 can generate a response (within responses 366) that includes an update to the current palatal expansion treatment plan. In embodiments, the update can be that the patient should be held at the current treatment stage and/or current palatal expander, or that the patient should revert back to a previous treatment stage and hold it there (e.g., until the patient is determined ready to proceed, until a new palatal expander with an acceptable expansion force can be manufactured), for example. In embodiments, the update can be a modification to a design of one or more palatal expanders to reduce the rotational force exerted on one or more teeth.

Data processing 3.2 can be used to determine a bite force measured by one or more force sensors and/or a number of bites measured by one or more force sensors. Determined observations 364 may include the bite force and/or number of bites, for example. Data processing 3.2 may also be used to perform a bite analysis of the patient to identify one or more bite contact points. The bite contact points may be compared to ideal bite contact points. If the identified bite contact points deviate from ideal bite contact points by more than a threshold amount, then one or more actions may be taken. Data processing 3.2 may also be performed to assess whether a patient suffers from bruxism and/or whether the patient performs nighttime clenching or grinding of their tooth (e.g., while sleeping).

In cases where observations 364 include an observation that the number of bites experienced by a palatal expander exceeds a threshold number of bites and/or that the bite force exceeds a bite force threshold, then the patient may be advised to change to a new palatal expander (e.g., a next palatal expander in a series of palatal expanders), such as by outputting a notification. In cases where the patient is identified as having bruxism, the patient and/or doctor may be notified of the bruxism and/or the patient may be advised to have a doctor visit. In some instances, the patient and/or doctor may be notified of a timing of the bruxism (e.g., that the bruxism occurs while the patient sleeps). In cases where the patient is identified as having bruxism, one or more new palatal expanders may be designed and manufactured that are thicker and/or composed of a harder material that is more resistant to wear caused by the bruxism. In cases where the patient is identified as having non-optimal bite contact points, one or more new palatal expanders may be designed and manufactured that adjust the thickness at one or more regions to modify the bite contact points and to cause the bite contact points to be closer to ideal.

In embodiments, data processing 3.2 can include performing analysis of image data (e.g., image processing) within data 362. For instance, in embodiments, data processing 3.2 can include performing image processing (e.g., optionally via a machine learning and/or AI model) to perform segmenting, feature extraction/recognition, tooth recognition, etc., on images or image data within data 362. Accordingly, data processing 3.2 can be capable of extracting or determining tooth positions, tooth orientations, spatial placements of teeth, spatial arrangements of various segments (e.g., dental structures identified from segmentation), placement and orientations of a palatal expander, a width of the patient's palate (e.g., arch width), orientations and identification of features (e.g., tissues, teeth, diastemas, crossbite characterization, discoloration, etc.), a facial width, a nose breadth, a facial asymmetry, and so on. Spatial arrangements of one or more segments (e.g., of teeth and/or other oral structures such as palatine rugae) may be determined based on segmentation of a current image. Similar segmentation may be performed of one or more prior images and/or a 3D model of a dental arch from a treatment plan. Comparisons of the spatial arrangements of the one or more dental objects may be made between the segmentation information of the current image and the segmentation information from the past image(s) and/or 3D model(s) from the treatment plan. Determined changes in spatial arrangements may be recorded as observations 364. Thus, through observations 364, data processing 3.2 can perform monitoring of at least the progress of palatal expansion of a patient during a palatal expansion treatment plan (e.g., progress monitoring 3.2A), to identify the occurrence of any adverse events during stages of the plan (e.g., event monitoring 3.2B), to determine the level of retention within a retention stage of the plan (e.g., retention monitoring 3.2C), to determine the effectiveness of the palatal expansion treatment plan and procedure at large, and/or to determine the amount of facial widening, nose widening, introduced facial asymmetry, and so on.

Progress monitoring 3.2A can be used to monitor the progress of a patient through the palatal expansion treatment plan. In embodiments, this can include monitoring an arch width of the patient. To monitor the arch width, data processing 3.2 can process an upper occlusal image of data 362 in tandem with the patient's palatal expansion treatment plan. Data processing 3.2 can be used to segment the occlusal image (e.g., via a feature extraction, or image segmentation model and/or algorithm), to segment the occlusal image into individual teeth. To determine the arch width between a set of teeth (e.g., between the first molars and/or the canines), the occlusal width of the relevant teeth of the treatment plan (e.g., in millimeters, as measured from a 3D model of the patient's upper dental arch) can be compared to their size in the image (e.g., in pixels). This comparison can be performed to produce a conversion factor F_{CON}, for converting between millimeters and pixels (or vice versa). For instance, in embodiments, the conversion factor F_{CON}, can indicate the real-world, or physical width of a pixel (e.g., in millimeters).

Since each image taken within image data of data 362 can be taken from a different camera angle, or perspective, in embodiments surveillance module can calculate F_{CON} individually for each image, and/or for different regions of each image. For instance, in embodiments, the conversion factor F_{CON} can be calculated via the equation

$${F}_{CON} = \frac{{RW}_{T}}{{PW}_{T}} ,$$

where RW_{T} is the real-world or physical occlusal width of a tooth in millimeters. PW_{T} can be the same occlusal width in pixels as seen in the image data. Thus F_{CON} can be generated to determine a size of a pixel in real-world measurements (e.g., in mm).

Once the conversion factor F_{CON} is computed for an image, the total arch width at the relevant tooth can then be determined using the pixel distance (e.g., number of pixels between two points as extracted from the image data), and multiplied by the conversion factor F_{CON}. In embodiments, conversion factor F_{CON} can then be used to generate distances and/or measurements from any feature within the image data, as desired. Alternatively, F_{CON} can be individually computed for each tooth as desired. In embodiments, other features, including any known distances apparent in the image data, can be used to generate F_{CON}. These can include, for example, size of teeth in one or more dimensions, size of the palatal rugae, etc.

To introduce further accuracy to measurements taken from image data of data 362, the angle at which an image sensor (e.g., a camera) is placed with respect to the teeth can be accounted for. For instance, in addition to, or alternatively to, conversion factor F_{CON}, a correction factor F_{COR} can be computed to account for the changes in physical size attributable to an angle that the capturing image sensor (e.g., a camera) was held at. In embodiments, this correction factor F_{COR} can be used for improving the conversion factor F_{CON}, the pixel size estimate, the arch width measurement, other measurements produced from the image data, and so on. Conversion factor F_{CON} and/or correction factor FOR can be determined and applied to any and all image data processed by data processing 3.2 operations.

Should an expansion of an arch width of the patient advance to such a level (e.g., to a predetermined threshold as defined by the palatal expansion treatment plan), data processing 3.2 can indicate such for response generation 3.3 as an observation (within observations 364).

Determined arch width in physical units of measurement may be compared to a planned arch width in physical units of measurement (e.g., mm, inches, etc.). A difference between planned and actual arch width can be determined based on the comparison. In cases where observations 364 includes an observation that the arch width has expanded to a sufficient level or threshold, response generation 3.3 can generate a response (within responses 366) that includes delivering a notification to a patient and/or HCP. The notification can include an indication of the level of the expanded arch width.

To monitor the progress of a patient through the palatal expansion treatment plan (e.g., progress monitoring 3.2A), data processing 3.2 can be performed to monitor a posterior crossbite of the patient (i.e., the occlusion of the buccal cusps of the upper molars is on the central fossae of their opposing lowers). Data processing 3.2 can process image data (of data 362), in tandem with the patient's palatal expansion treatment plan. In embodiments, one or more anterior images, and/or lateral images of the patient's dentition can be used. Data processing 3.2 can begin such a process by segmenting the image data (e.g., via a feature extraction, or image segmentation model and/or algorithm) into individual teeth.

When using the anterior image, (e.g., after image segmentation to identify teeth), individual teeth (e.g., molars or premolars) can be compared to characterize a level of posterior crossbite. For instance, the buccal edge of the maxillary molars can be compared to the buccal edge of the mandibular molars. Where the mandibular molars are more buccal than the maxillary molars, the patient can be exhibiting posterior crossbite. When maxillary molars are more buccal than the mandibular molars over a series of images taken at different times during treatment, this can indicate that the crossbite has been corrected. Based on the comparative sizes of the visible portions of the maxillary molars and the mandibular molars, processing logic may determine whether a posterior crossbite has improved and/or has been corrected.

In some embodiments, correction of posterior crossbite of a patient can be an indication that the expansion has sufficiently widened the arch and that further stages of the palatal expansion treatment plan can be forgone. Should the correction of posterior crossbite advance to such a level (e.g., to a predetermined threshold for posterior crossbite correction), data processing 3.2 can indicate such for response generation 3.3 as an observation (within observations 364).

In cases where observations 364 includes an observation that the correction of posterior crossbite advance to such a level, response generation 3.3 can be performed to generate a response (within responses 366) that includes delivering a notification to a patient and/or HCP. The notification can include an indication of the level of correction of posterior crossbite.

To monitor the progress of a patient through the palatal expansion treatment plan (e.g., in one or more expansion stages of the plan), data processing 3.2 can be performed to monitor the creation and/or widening of a diastema of the patient. For example, skeletal expansion may be accompanied by the creation and/or widening of a diastema between the maxillary central incisors. Data processing 3.2 can accordingly include monitoring for the presence and/or size of the diastema using anterior image data (e.g., open or closed bite anterior images) of data 362. For instance, such image data can first be segmented into individual teeth and the distance between the left and right central incisors measured in pixels. The pixel size of the distance can be found (e.g., via edge detection algorithms) and converted from pixels to millimeters (e.g., via the methods described above with respect to arch width monitoring). In embodiments, this approach can further be used in monitoring an orthodontic treatment intended to close a diastema.

Should a diastema be created, or an existing diastema advance to a threshold level (e.g., to a predetermined threshold as established by the palatal expansion treatment plan), data processing 3.2 can indicate such to response generation 3.3 as an observation (within observations 364).

In cases where observations 364 includes an observation that a diastema be created, or an existing diastema advance to such a level (e.g., to a predetermined threshold as established by the palatal expansion treatment plan), response generation 3.3 can generate a response (within responses 366) that includes delivering a notification of such to a patient and/or HCP.

Data processing 3.2 can be performed to monitor the palatine rugae of a patient's oral cavity. For example, frequently, skeletal expansion is accompanied by changes to the palatine rugae. By monitoring changes in the palatine rugae of a patient, the processing logic can differentiate between palatal widening and dental expansion (i.e., determine if the palate is widening or if the dentition is moving/tilting). If the separation between molars is increasing, but the palatal rugae are not changing, this may indicate that the teeth are tipping outward and that the patient's palate is not expanding. However, if the separation between molars is increasing and the palatal rugae are changing, this may indicate that separation between molars is caused by skeletal changes (e.g., that the palate is expanding).

To monitor the palatine rugae of a patient, data processing 3.2 can include first processing an upper occlusal image of data 362. The outlines and/or contours of the rugae can be identified (e.g., using segmentation, edge detection, contour detection, color references, etc.), optionally together with one or more features of the patient's upper dental arch. The identified rugae contours can then be aligned, and compared, to an initial, or previous, set of rugae contours. Changes in the rugae contours can indicate that palatal expansion is occurring, while lack of changes, or static, rugae contours can indicate that dental expansion is occurring. Changes in the rugae contours can include changes in distance between rugae contours, changes in distance between one or more points on one or more rugae contours and one or more other features (e.g., teeth) of the dental arch, and so on.

In some embodiments, comparison of image data of rugae contours can be accomplished by comparing a contour distance, or a longitudinal distance of the rugae contours to one another. For example, once the current rugae contours have been aligned to the initial, or previous, set of contours, the contour distance between rugae contours can be found. As mentioned, contour distance can increase with the amount of palatal (skeletal) expansion.

Multiple approaches can be used to align the rugae contours, including, but not limited to, identifying the incisive papilla through object detection and using it as a reference point to align the rugae contours. Other approaches can include computing and/or optimizing an affine transform (e.g., a homography) that best aligns the current and previous/initial rugae contours. In embodiments, the transformation can be found using matching points from different sets of image data, or by optimizing over a distance metric (e.g., between two rugae contours of different sets of image data).

A variety of distance metrics can be used both for assessing the change in rugae and/or for computing an optimal transform. In one embodiment, a Hausdorff distance or a modified (one-way) Hausdorff distance can be found. Other options can include identifying matching points (e.g., using biological reference points or image processing techniques such as SIFT, SURF, or ORB, etc.) and/or computing a mean distance between the matching points.

In embodiments, a subset of the rugae contours are used to optimize the transform between the images (e.g., such as a subset of points lying on a particular plane). In embodiments, additional variations can include using a subset of the rugae contours to measure the contour distance.

Should changes in the palatine rugae be detected to a sufficient level (e.g., to a predetermined threshold as established by the palatal expansion treatment plan), or should palatine rugae changes fail to meet such a sufficient level, data processing 3.2 can indicate such to response generation 3.3 as an observation (within observations 364).

In cases where observations 364 include an observation that sufficient changes (or lack thereof) in the palatine rugae have been detected, response generation 3.3 can generate a response (within responses 366) that includes delivering a notification of such to a patient and/or HCP.

Data processing 3.2 can include monitoring a fit of a palatal expander through image data of data 362. To monitor the device fit of a palatal expander, image data of a patient wearing the palatal expander can be processed. From such images, fit issues such as gaps between the occlusal surfaces of the molars and the palatal expander can be detected. For example, in embodiments, a potential cause of low or insufficient device fit can be a missing retention attachment. This can be monitored through the processed image data.

Should the fit of a palatal expander be deemed insufficient (e.g., to a predetermined threshold as established by the palatal expansion treatment plan), data processing 3.2 can indicate such to response generation 3.3 as an observation (within observations 364). In cases where observations 364 include an observation that a fit of a palatal expander is deemed insufficient, response generation 3.3 can generate a response (within responses 366) that includes delivering a notification of such to a patient and/or HCP.

Data processing 3.2 can be used to determine if an amount of rotation between a first palatal region (e.g., left side) and a second palatal region (e.g., right side) of a patient's palate is within an acceptable range based on processing of image data. In an example, an amount of rotation or angle of a left side of the patient's palate relative to a right side of the patient's palate may be measured and compared to planned rotation/angle. In some embodiments, it may be determined that an amount of rotation between a first palate region and a second palate region deviates from a planned amount of rotation by more than a threshold amount. In response to this determination, a treatment plan may be altered in embodiments. For example, a treatment plan may be altered to speed up treatment (e.g., by reducing the amount of time the patient is required to wear a palatal expander of an upcoming stage or a set of palatal expanders of a set of upcoming stages). As another example, a treatment plan may be altered to slow down treatment (e.g., by increasing the amount of time the patient is required to wear a palatal expander of an upcoming stage or a set of palatal expanders of a set of upcoming stages). These alterations may occur dynamically and may occur continually based on continued measurements as a patient's treatment progresses. It may be difficult to predict at the beginning of treatment how a patient responds to treatment; so continued monitoring and alteration of the treatment plan as disclosed herein allows for optimizing the patient's treatment for the duration of the treatment to allow for fast, safe, and effective treatment.

Event monitoring 3.2B can be performed by and/or during data processing 3.2. This can include monitoring adverse events via processing of image data and/or 2D images (e.g., to detect soft tissue damage, intrusion of support teeth, etc.). For instance, in embodiments, processing logic can monitor soft tissue damage from one or more of an occlusal, anterior, and/or lateral image of data 362. As described, data processing 3.2 can perform processes such as object detection and/or classification to identify images or regions of images exhibiting soft tissue damage (e.g., such as ulceration on the gingiva, palatal surface, or buccal mucosa, etc.). Such damage (e.g., ulceration on the gingiva) can be apparent in image data such as lateral images. For example, ulceration of the palatal surface should be apparent from occlusal images, and ulceration of the buccal mucosa can be visible on anterior images.

In some embodiments, an adverse event is determined based on an observation of a change in spatial arrangement of one or more segments (e.g., of teeth) between a current treatment stage and a past treatment stage. In some embodiments, a change or difference in a spatial arrangement of one or more dental objects is determined based on a comparison of a spatial arrangement of a plurality of segments (e.g., dental objects) of a current image to an expected spatial arrangement of the plurality of segments for the current stage of treatment. The change or difference in the spatial arrangement may be unexpected and/or unplanned, and may not be a desired outcome. For example, the change in spatial arrangement may indicate a buccal tipping of one or more molars, a formation of a malocclusion, and so on.

In embodiments, object detection and classification can be based on simple color deviations or more complicated computations (e.g., feature detection), including pre-trained machine learning models that output detections of soft tissue damage.

Should a sufficient level of soft tissue damage be detected, data processing 3.2 can indicate such for response generation 3.3 as an observation (within observations 364). In cases where observations 364 include an observation that a sufficient level of soft tissue damage has occurred, response generation 3.3 can generate a response (within responses 366) that includes delivering a notification of such to a patient and/or HCP.

Data processing 3.2 can include monitoring for the intrusion of supporting teeth from one or more images within data 362. In embodiments, data processing 3.2 can be performed to detect intrusion by measuring the height of the clinical crowns of the supporting teeth in a processed image. Such measurements can be determined using as has been previously described.

Should a sufficient level of intrusion of supporting teeth (e.g., to a predetermined threshold as established by the palatal expansion treatment plan) be detected, data processing 3.2 can be performed to indicate such to response generation 3.3 as an observation (within observations 364). In cases where observations 364 include an observation that a sufficient level of intrusion of supporting teeth has occurred, response generation 3.3 can be performed to generate a response (within responses 366) that includes delivering a notification of such to a patient and/or HCP.

Data processing 3.2 can be performed to monitor buccal tipping of molars from one or more images within data 362. In embodiments, processing logic can detect excessive buccal tipping of molars. For instance, using the segmentation of an anterior image, the amount of buccal tipping of the maxillary molars can be found by determining the angle of the teeth in the image. The image segmentation of a single molar can be indicated by a curved surface that is generally pointing in the negative-y direction (in anterior image space), but a buccally tipped expander molar will be less downward pointing. Buccally-tipped molars can be more outward pointing, with respect to a center of the oral cavity.

Should a sufficient level of buccal tipping of molars (e.g., to a predetermined threshold as established by the palatal expansion treatment plan) be detected, data processing 3.2 can be performed to indicate such to response generation 3.3 as an observation (within observations 364). In cases where observations 364 include an observation that a sufficient level of buccal tipping of molars has occurred, response generation 3.3 can generate a response (within responses 366) that includes delivering a notification of such to a patient and/or HCP.

Data processing 3.2 can include monitoring for anterior and/or posterior tissue impingement from one or more images within data 362. For instance, data processing 3.2 can be performed to detect tissue impingement in image data as a line at the back of the palate. This can be detectable in an occlusal image. Detection can thus be performed by means of object detection, semantic segmentation, or standard edge detection (and so on) in the image.

Should a sufficient level of anterior and/or posterior tissue impingement (e.g., to a predetermined threshold as established by the palatal expansion treatment plan) be detected, data processing 3.2 can be performed to indicate such to response generation 3.3 as an observation (within observations 364). In cases where observations 364 include an observation that a sufficient level of anterior and/or posterior tissue impingement has occurred, response generation 3.3 can generate a response (within responses 366) that includes delivering a notification of such to a patient and/or HCP.

Data processing 3.2 can include monitoring for the eruption of missing teeth and/or exfoliation of current teeth from one or more images within data 362. For instance, for patients in their later dentition, both eruption of missing teeth and exfoliation of current teeth is possible. Data processing 3.2 can be performed to detect such through image segmentation from any of the standard perspective of intraoral images (e.g., within data 362). In embodiments, exfoliation can be detected as a tooth that is no longer present in the image when compared to historical, or previous, images. Such a tooth can be present in the treatment plan. In embodiments, eruption can be detected as a “new” tooth in an image without a corresponding tooth in the treatment plan. In addition, or in alternative, to direct segmentation, eruption and/or exfoliation can be identified by comparing an initial or previous image to the current image, potentially through the use of an image transformation, as described above (e.g., regarding changes in the palatal rugae).

Should a sufficient level of eruption of teeth, “new” teeth, exfoliation of current teeth be detected, data processing 3.2 can indicate such for response generation 3.3 as an observation (within observations 364). In cases where observations 364 include an observation that a sufficient level of eruption teeth or exfoliation of current teeth has occurred, response generation 3.3 can generate a response (within responses 366) that includes delivering a notification of such to a patient and/or HCP.

Retention monitoring 3.2C can be performed by and/or during data processing 3.2. During such, observations (within observations 364) that can be monitored through the processing of image data by data processing 3.2 can include aspects and events of a retention stage (e.g., following an expansion stage of the palatal expansion treatment plan). For instance, as previously discussed, once the expansion has completed, the patient can enter into a retention or holding stage where the expansion gains are consolidated. During the retention stage, several aspects of the patient's dentition can be monitored.

Data processing 3.2 can include monitoring the retention of arch width (e.g., arch width retention) from one or more images within data 362. For instance, in embodiments, data processing 3.2 can be performed to monitor arch width retention in the same or similar way that arch width is monitored in the expansion progress tracking.

Should arch width during a retention stage change or alter to a sufficient level (e.g., to a predetermined threshold as established by the palatal expansion treatment plan), data processing 3.2 can be performed to indicate such to response generation 3.3 as an observation (within observations 364). In cases where observations 364 include an observation that a sufficient level of change in arch width has occurred, response generation 3.3 can generate a response (within responses 366) that includes delivering a notification of such to a patient and/or HCP.

Data processing 3.2 can include monitoring the tipping of the molars from one or more images within data 362. For instance, molar tipping can occur if there is a relapse of the palatal expansion, but the retention device can continue to hold the molars in place. In embodiments, molar tipping can be monitored in same way that excessive buccal tipping of molars is monitored (e.g., as described above).

Should molar tipping during a retention stage occur to a sufficient level (e.g., to a predetermined threshold as established by the palatal expansion treatment plan), data processing 3.2 can be performed to indicate such to response generation 3.3 as an observation (within observations 364). In cases where observations 364 include an observation that a sufficient level of molar tipping has occurred, response generation 3.3 can be performed to generate a response (within responses 366) that includes delivering a notification of such to a patient and/or HCP.

Data processing 3.2 can include monitoring a discoloration of the palatal expander from one or more images within data 362. Excessive amount of strain on the palatal expander may cause a color or transparency level of the palatal expander to change. For instance, discoloration of the palatal expander can indicate that the device is being stressed or strained in an unexpected manner. Through comparison of a current image of the retention device to a prior image of the retention device and/or to a known color palate (e.g., indicating forces/pressures associated with different colors or other visual indications), color changes can be detected. Recognizing that image color and white balance can play a role in the absolute color of the palatal expander in an image, in embodiments, a color comparison can be performed by using tooth color and/or the gingival color as reference. By aligning reference colors to be equivalent between images, a comparison can be made of the device color. Alternatively, reference color and/or point can be taken using a portion of the palatal expander that is less subject to discoloration. In some cases, changes in device color (e.g., discoloration) can indicate that the patient's expansion is relapsing. In embodiments, such an approach to characterizing device discoloration can also be used during expansion progress monitoring (e.g., as described above) to identify unusual stresses or strains on the palatal expander. These forces can imply that the skeletal changes are not keeping up with the staging and that the staging should be slowed.

Should device discoloration during a retention stage occur to a sufficient level (e.g., to a predetermined threshold as established by the palatal expansion treatment plan), data processing 3.2 can indicate such to response generation 3.3 as an observation (within observations 364). In cases where observations 364 include an observation that a sufficient level of device discoloration has occurred, response generation 3.3 can generate a response (within responses 366) that includes delivering a notification of such to a patient and/or HCP.

Data processing 3.2 can include synthesizing one or more images showing a predicted future face of a patient. One or more 2D images of a face of the patient may be captured before dental treatment and during one or more intermediate stages of the dental treatment. The one or more 2D images of the current face of the patient may be captured at a current treatment stage, and may be input into a machine learning model (e.g., a generative model such as a generator of a generative adversarial network (GAN)) trained to output predicted future facial images. In embodiments, the one or more 2D images of the current face are input into the machine learning model together with a stage number of the current stage of treatment. Additionally, one or more prior 2D images of the patient's face may be input into the machine learning model with indications of stage numbers associated with the one or more prior 2D images. Additionally, or alternatively, a total number of stages of the treatment plan may be input into the machine learning model. Based on the input data, the machine learning model may output the one or more synthesized predicted images of the predicted future face of the patient. Image processing (optionally using machine learning) may be performed on the one or more synthesized images to determine one or more observations of the post-treatment face of the patient in embodiments. In some embodiments, the machine learning model that outputs the predicted images also determines the one or more observations. Alternatively, a separate machine learning model may process the predicted images to generate the one or more observations. The observations may include a measurement of a facial width, a measurement of a nose breadth and/or a measurement of a facial asymmetry in embodiments. These measurements may be compared to pre-treatment measurements and/or measurements of the current or past facial images captured during treatment for facial width, nose breadth and/or facial asymmetry in embodiments. Such measurements may be determined by processing the captured facial images using the same machine learning model that processes the predicted future facial images in some embodiments.

In some embodiments, the one or more images are annotated based on one or more observations (e.g., by drawing lines and/or measurements for a facial width, nose breadth, facial asymmetry, etc. on the image(s). In some embodiments, one or more image overlays may be generated that are added to the predicted facial image(s) of the patient that show an amount of change in the facial width, nose breadth and/or facial asymmetry. In some embodiments, a video is generated that shows the progression of the patient's face from pre-treatment to post-treatment. The video may be generated by adding the pre-treatment, intra-treatment and predicted post-treatment images together. In some embodiments, one or more of the images may be modified to cause the patient's face to have the same pose, size, shading, etc. in each of the images. In some embodiments, one or more additional facial images is generated for time periods between existing facial images. Such additional facial images may be generated by interpolating between existing facial images in embodiments. The video showing the progression of the facial shape change of the patient may be sent to the patient and/or doctor for display. In some embodiments, the video is generated using the technology set forth in U.S. application Ser. No. 18/525,530, entitled “Augmented Video Generation with Dental Modifications”, filed Nov. 30, 2023, which is incorporated by reference herein in its entirety.

An image of the predicted-post treatment face of the patient, measurements of the predicted post-treatment face, and/or a video showing a gradual change of the patient's face between a pre-treatment condition and a predicted post-treatment condition may be generated and sent to a device of a patient and/or a doctor. The patient may determine that they do not like how their face is predicted to look after treatment, and may provide feedback to the doctor to change the treatment plan. Example changes to the treatment plan may include adjusting the treatment plan to ameliorate any predicted facial asymmetry, reducing a number of stages of the treatment plan to reduce a total amount of palatal expansion (e.g., shortening treatment) and/or halting palatal expansion treatment, for example. In some cases, if a predicted amount of facial asymmetry is determined, a recommendation to adjust the treatment plan and/or to perform surgery may be generated and provided to the patient and/or doctor. For example, one or more new palatal expanders that will replace one or more previously designed palatal expanders may be determined that have a different thickness, different rigidity and/or different materials at one or more locations to cause forces exerted on the patient's palate to be changed. For example, one or more palatal expanders may be redesigned to exert asymmetry forces on the patient's palate to counteract a facial asymmetry that is observed or predicted. In another example, a recommendation to perform surgery to loosen one or more sutures of a palate of the patient may be generated, where the surgery may counteract a predicted facial asymmetry.

In embodiments, process 300 can be repeated, and data 362, observations 364, and responses 366 can be generated more than once. For instance, in some cases, during a palatal expansion treatment plan (e.g., plan 100 of FIG. 1), process 300 can be repeated multiple times during each stage. In embodiments, process 300 can be repeated at a checkpoint (e.g., a predefined point in time, at a planned transition point between stages, etc.). Accordingly, responses 366 can be generated with respect to a stage, to more than one stage, to a current stage, and/or the treatment plan at large.

In some embodiments, the functions of process 300 can be accomplished by any combination of human and machine components. For example, in some embodiments, a software program can process the collected data 362 to extract observations 364 into a different form (e.g., a 3D model that is formed from image data, a “heat map” characterizing palatal expansion, tooth movement, forces, etc.) and then a human (e.g., an HCP) can produce responses 366 from observation 364. In further embodiments, a machine-made judgement (e.g., a response within responses 366) and the associated data can be presented to an HCP to simply confirm, or reject, the machine-made judgement or assessment. In embodiments, any mixture of human and software elements can be combined. Such an implementation of process 300 can thus leverage strengths from both human and machine capabilities.

In some embodiments, the machine element associated with the process 300 can be foregone, and an HCP can be directly shown the collected data 362. In such cases, the HCP can solely assess and judge whether the patient should advance to the subsequent stage, or remain at the current stage for additional time. Alternatively, in embodiments, responses 366 can be produced and executed directly from the observations 364 by a machine-learning (or similar) model. In such embodiments, process 300 can be completely automated, and performed by any kind of computer algorithm (e.g., a machine-learning model, an analytical model, a loss-optimization function, etc.).

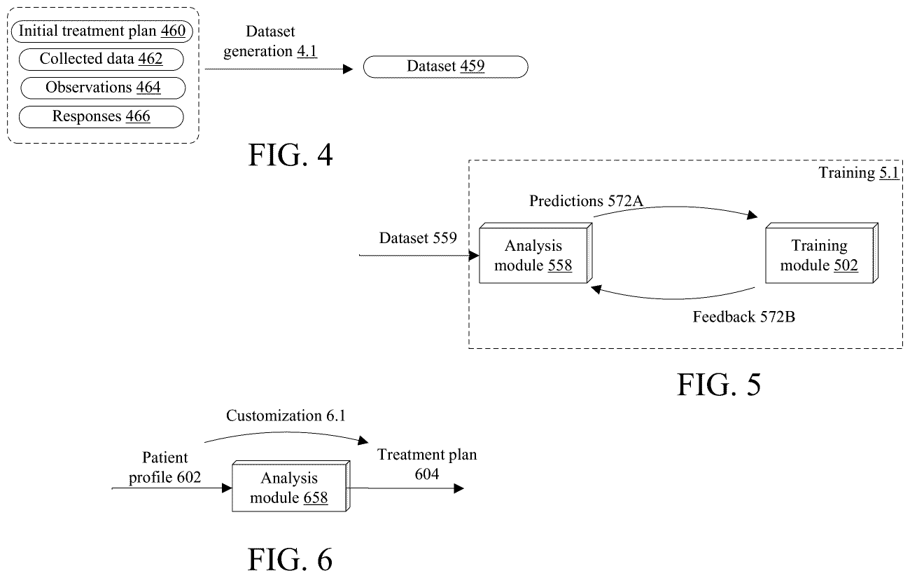

FIG. 4 illustrates an example dataset generation process capable of being performed by processing logic via data captured during patient treatment according to the palatal expansion treatment plan of FIG. 1, in accordance with some embodiments of the present disclosure. The dataset that is output from this process may be used to train a model (e.g., a machine learning model) that can then be used to generate treatment plans for new patients, to modify existing treatment plans, and so on. For example, in some embodiments, the dataset may be used in the training process described below with reference to FIG. 5.

In some embodiments, dataset generation 4.1 can be a process performed by a dataset generator (e.g., of the holistic monitor of the system as described with respect to FIG. 14). Dataset generation 4.1 may include operations performed by one or more computing devices, which may be client devices and/or server devices, for example. One or more operations of dataset generation 4.1 may be performed by a processing logic, which may include software, firmware, or a combination thereof that executes on one or more processors.

For example, dataset generation 4.1 can be performed by combining data items for one or more completed palatal treatments to form a dataset 459 that may be used to train a machine learning model. For example, dataset generation 4.1 may include generating a data item (which may represent a particular patient case) that may include an initial treatment plan 460, collected data 462 that was collected during treatment, observations 464 associated with the treatment, and responses 466 (e.g., that may have been output or performed during treatment, such as by a doctor and/or processing logic). In embodiments, the dataset generator 456 can combine information from treatments of multiple patients to form a dataset 4591. In embodiments, the collected data 462 and/or observations 464 can be labeled as “inputs,” within dataset 459. In embodiments, the responses 466 and/or observations 464 can be labeled as correlated “outputs.” With respect to training a machine learning model to generate predicted future images (e.g., of the patient's face), inputs can be pre-treatment facial images and/or intra-treatment facial images (optionally together with treatment plan information such as number of stages, stage(s) associated with input images, etc.), and outputs can be post-treatment facial images.

In embodiments, dataset 459 can be an aggregate of data from multiple patients and multiple sampling times. For example, in embodiments, dataset 459 can include collected data 462 from a patient taken from multiple stages along that given patient's palatal expansion treatment plan. In embodiments, dataset 459 can contain collected data 462 from multiple (including any number of) patients as well. In embodiments, the data can be discretized according to data segments including related collected data 462, observations 464, and responses 466, corresponding to a checkpoint and/or treatment stage, and/or patient. In embodiments where dataset 459 includes various types of data, data segments of dataset 459 can be categorizable by patient type and/or patient characteristics (e.g., age, demographics, etc.), etc.

In embodiments, dataset 459 can contain one or more data segments of data that corresponds to a granularized, specific portion or stage of a palatal expansion process. For example, in embodiments, the dataset 459 can contain a data segment (i.e., collected data 462, observations 464, and/or responses 466) that can be organizable according to any stage, arch width, tooth position, patient demographics, etc., associated with a palatal expansion treatment plan. In some embodiments where dataset 459 has been further granularized, process 4.1 can produce multiple, including any number of, datasets 459, corresponding to multiple, or any number of, specific aspects of a palatal expansion treatment plan.

In some embodiments, dataset generation 4.1 can be performed to output the dataset 459 for further processing. In embodiments, such further processing can include advanced data analysis, training of a machine learning module, segmentation, or modeling of the data, and so on.

FIG. 5 illustrates an example training process (training 5.1) for training an analysis module 558 via the dataset of FIG. 4, in accordance with some embodiments of the present disclosure. Training 5.1 may include operations performed by one or more computing devices, which may be client devices and/or server devices, for example. One or more operations of training 5.1 may be performed by a processing logic, which may include software, firmware, or a combination thereof that executes on one or more processors.

In embodiments, dataset 559 can be modified, and relevant data extracted before input to analysis module 558. In embodiments where dataset 559 corresponds to a single procedure, or where multiple datasets are generated each corresponding to a single procedure, the data can be analyzed by analysis module 558 to further characterize the procedure without modification. In cases where multiple procedures are contained within a stored dataset, the data segments can be categorized, and data segments corresponding to a single procedure can be extracted for processing.

In embodiments, where further levels of granularity are desired, the plan dataset can be categorized and the plan dataset can be “focused,” to retain data segments corresponding to the desired level of granularity. For example, within a general orthodontic procedure, aligners can be designed to apply specific manipulations to one or more teeth. These manipulations can include, tipping, torquing, rotating, extruding, retracting, protracting, etc. In embodiments, dataset 559 can be focused (e.g., only include the data segments) to include only collected data and plan updates for a single manipulation, for a single tooth, and the produced feature estimates can correspond to such a specific dental procedure.

Accordingly, in embodiments, the plan data can be aggregated and analyzed to produce feature estimates, generalizations, characterizations, estimates, or insights, etc., based on the data for a specific procedure, manipulation, or element, as described above. For example, in embodiments in which dataset 559 is manipulated to contain data which corresponds to movement of a specific tooth, dataset 559 can be processed and analyzed to generate feature estimates and characterizations for that movement. Such estimates and characterizations can include features such as estimated average procedure duration, estimated average procedure difficulty, identification of subprocesses that can be required for the overall procedure, etc. In embodiments, such estimates and characterizations can be generated for a given movement, a given tooth, a given patient data (e.g., biology, age, etc.), or any other influencing factor that has been captured within collected data within the plan dataset, according to any level of granularity.

In some embodiments, such feature estimates can be based on a wide array of qualitative and quantitative input metrics (e.g., parameters within collected data). For instance, the level of difficulty of the procedure can be estimated or assessed. In embodiments, for example, the estimated length of time for the procedure can be calculated based on historical data from similar cases, the complexity of similar procedures, the historical responsiveness of individual teeth to orthodontic forces for such a procedure, etc.

Furthermore, in embodiments, dataset 559 can also include data related to the estimated patient discomfort or pain levels, anticipated need for adjunctive procedures (e.g., extractions, bone grafts, etc.), as well as the expected aesthetic outcomes.

In some embodiments, such feature estimates and characterizations can be dynamically updated to reflect real-time progress, facilitating more accurate planning and adjustment of ongoing treatments.

In some embodiments, the generation of feature estimates, and the functions of analysis module 558 can be accomplished by a human, a computer or software module, or any combination of the two. For instance, in embodiments, an HCP or similarly trained dental professional can analyze the plan dataset to generate feature estimates or generalization for a given procedure. In alternate embodiments, a software program, mathematical model, or any similar computer program can accomplish the functions of analysis module 558.

In some embodiments, analysis module 558 may comprise a machine learning model that has been or is being trained using one or more datasets 559. In embodiments, the machine learning model can be trained to generate predictions 572A, which may include estimations and/or classifications. These predictions may include plan updates and/or new palatal expansion treatment plans. The machine learning model may additionally or alternatively be trained to output notifications to be sent, for example, to client devices (e.g., a mobile phone of a patient). The predictions and/or other outputs of the machine learning model can be based on elements (e.g., data, observations, responses, etc.) within dataset 559. In embodiments, the machine learning model can be any kind of machine learning model (e.g., such as a neural network, a support vector machine, a large language model, and so on). In some embodiments, the training process seen in FIG. 5 can be repeatable for varying types of procedures, and more than one machine learning model can be used to accomplish the functions of the analysis module 558.

In embodiments, a large corpus of training data (including dataset 559) is used to train the machine learning model. Based on such data, the machine learning model can be trained to receive patient information (e.g., a 3D model of a patient's current upper and lower dental arches, patient age, patient gender, patient demographics information, etc.) and output a new treatment plan prior to treatment. Because the machine learning model has been trained on data showing not only treatment plans for patients and final outcomes for those patients, but also intermediate outcomes for those patients during various stages of treatment, the treatment plans output by the machine learning model can have increased accuracy. Additionally, or alternatively, the machine learning model can be trained to generate recommendations for existing treatment plans and/or updates to existing treatment plans based on an input of an already generated treatment plan and/or patient data gathered at a current stage of treatment and/or prior stages of treatment for the treatment plan. The machine learning model can output changes to lengths of treatment for one or more stages of treatment, can add additional stages of treatment, can remove stages of treatment, can otherwise modify existing stages of treatment, and so on. For example, the machine learning model can be trained to perform any of the estimates or characterizations described above (e.g., with respect to FIG. 3), including summarization of data related to one or more procedures, production of estimates for procedure duration, difficulty, required subprocess, etc.

In some embodiments, the training module 502 can access the dataset 559, can deliver collected data to the machine learning model as inputs, and can iteratively provide feedback 572B to the predictions 572A generated by the machine learning model. For example, for each iteration of training, an instance, or data point, can be fed as input into the machine learning model, prompting the machine learning model to produce an output. This output (e.g., the predicted plan updates and/or data analytics corresponding to such an input) can then be compared against the correct output from dataset 559. Any discrepancies between the machine learning model's output and the correct output can be noted, and feedback can be provided to the model to minimize such discrepancies in future iterations (e.g., by performing backpropagation). Such an iterative, continuous feedback loop can continue until a certain level of accuracy is reached by the prediction of the machine learning model.

In embodiments, after training, the machine learning model can be tested on a validation dataset, to ensure accuracy level are sufficient to deploy the machine learning model.

In some embodiments, such a machine learning model can vastly increase the efficiency of the system, and the production of plan updates and/or data analytics. For example, in some embodiments, the machine learning model can be a comprehensive and intricate machine learning model that has been extensively trained on vast and diverse data. Accordingly, the machine learning model can possess broad knowledge and understanding of patient data including images, sensor data, text data, and any other types of data within collected data within the dataset 559.

In some embodiments, a machine learning model is trained to generate synthetic images of a predicted future face of a patient. The machine learning model may be, for example, a generative model such as a GAN. A GAN is a class of artificial intelligence system that uses two artificial neural networks contesting with each other in a zero-sum game framework. The GAN includes a first artificial neural network that generates candidates (e.g., for post-treatment faces of patients) and a second artificial neural network that evaluates the generated candidates. The GAN learns to map from a latent space to a particular data distribution of interest (a data distribution of changes to input images that are indistinguishable from photographs to the human eye), while the discriminative network discriminates between instances from a training dataset and candidates produced by the generator. The generative network's training objective is to increase the error rate of the discriminative network (e.g., to fool the discriminator network by producing novel synthesized instances that appear to have come from the training dataset). The generative network and the discriminator network are co-trained, and the generative network learns to generate images that are increasingly more difficult for the discriminative network to distinguish from real images (from the training dataset) while the discriminative network at the same time learns to be better able to distinguish between synthesized images and images from the training dataset. The two networks of the GAN are trained once they reach equilibrium. The GAN may include a generator network that generates artificial intraoral images and a discriminator network that segments the artificial intraoral images. In embodiments, the discriminator network may be a MobileNet.

In one embodiment, one or more machine learning model is a conditional generative adversarial (cGAN) network, such as pix2pix. These networks not only learn the mapping from input image to output image, but also learn a loss function to train this mapping. GANs are generative models that learn a mapping from random noise vector z to output image y, G: z→y. In contrast, conditional GANs learn a mapping from observed image x and random noise vector z, to y, G: {x, z}→y. The generator G is trained to produce outputs that cannot be distinguished from “real” images by an adversarially trained discriminator, D, which is trained to do as well as possible at detecting the generator's “fakes”. The generator may include a U-net or encoder-decoder architecture in embodiments. The discriminator may include a MobileNet architecture in embodiments. An example of a cGAN machine learning architecture that may be used is the pix2pix architecture described in Isola, Phillip, et al. “Image-to-image translation with conditional adversarial networks.” arXiv preprint (2017).

FIG. 6 illustrates an example plan customization process 6.1 for customizing the palatal expansion treatment plan of FIG. 1, in accordance with some embodiments of the present disclosure.

Analysis module 658 can perform a customization process 6.1, to extract insights and generate a customized treatment plan 604 (e.g., which can be updates to an existing initial treatment plan) from a patient profile 602. Customization process 6.1 may include operations performed by one or more computing devices, which may be client devices and/or server devices, for example. One or more operations of customization process 6.1 may be performed by a processing logic, which may include software, firmware, or a combination thereof that executes on one or more processors. In some embodiments, predictions output by a machine learning model in FIG. 6 can be applied to a palatal expansion procedure according to varying levels of granularity, ranging from an overall holistic procedure adjustment to adjustments applicable to a specific stage, or palatal expander.

As discussed, analysis module 658 can perform a customization operation (e.g., “Customization 6.1”) and produce a treatment plan 604, or treatment plan updates. In embodiments, analysis module 658 corresponds to a trained version of analysis module 558, and may include one or more trained machine learning models. In embodiments, such produced plans or updates can include modifications made to the stages of an initial treatment plan. In embodiments, such produced plans or updates can be based on sensor data, image data and/or patient demographics, such as biological age, dental age, sex, ethnicity, genetic profiles, and so on.

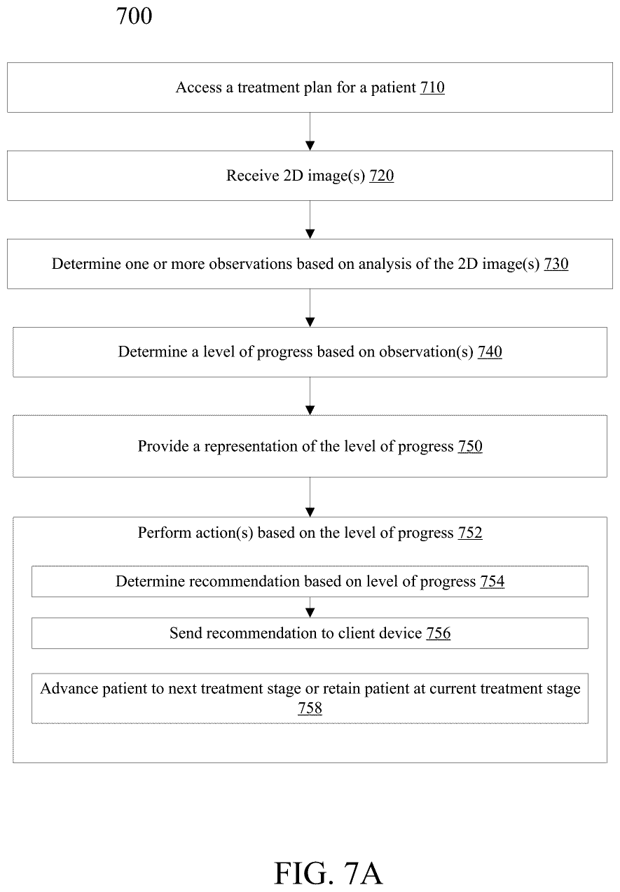

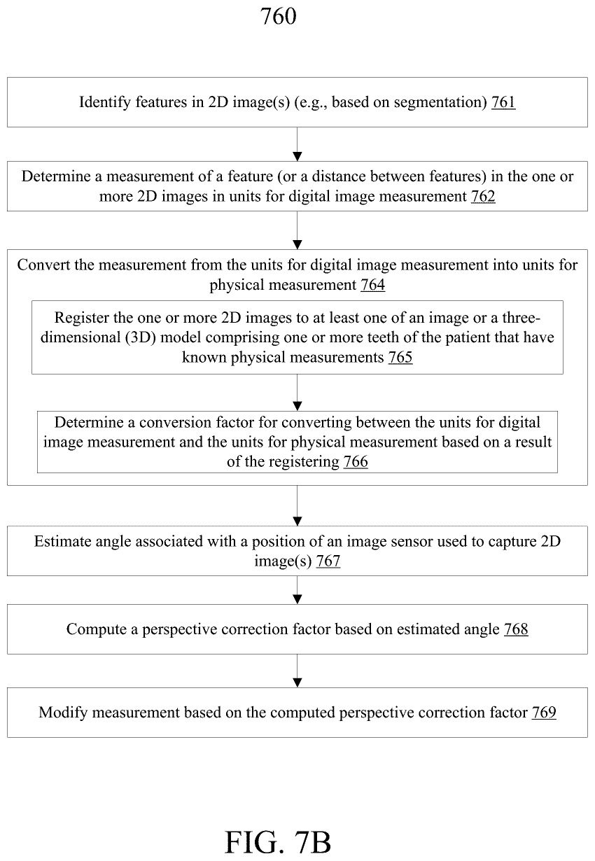

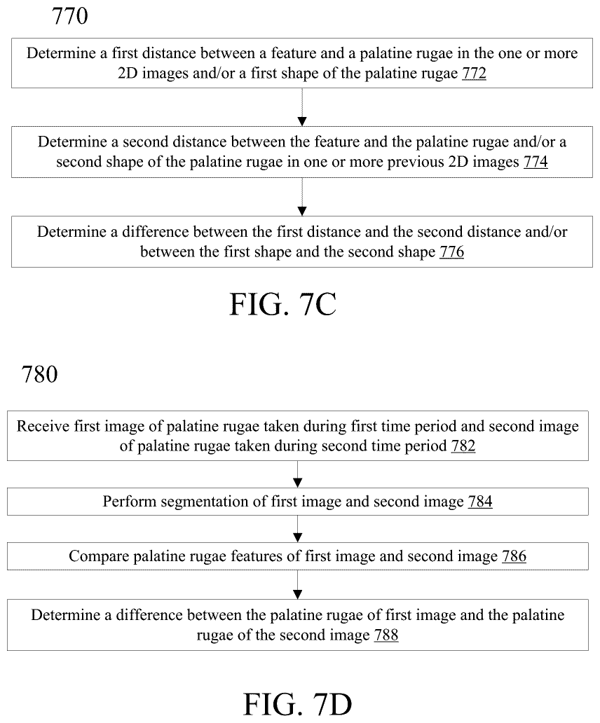

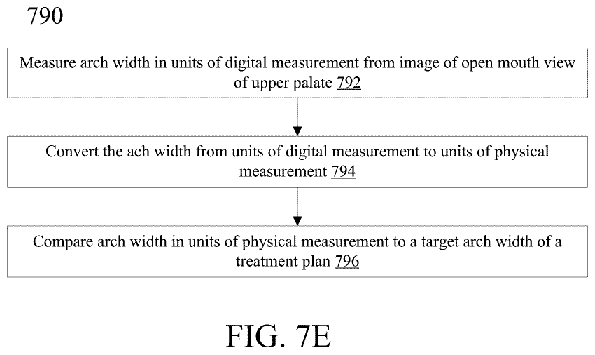

FIGS. 7A-11 illustrate flow diagrams of methods performed by a virtual dental care system, in accordance with embodiments of the present disclosure. These methods may be performed by processing logic that comprises hardware (e.g., circuitry, dedicated logic, programmable logic, microcode, etc.), software (such as instructions run on a processing device), or a combination thereof. In one embodiment, processing logic corresponds to a computing device of a holistic monitor 1450, treatment plan coordination platform 1420 and/or storage platform 1440 of virtual care system 1470 of FIG. 14.

FIG. 7A illustrates a flow diagram of an example method 700 for monitoring palatal expansion, in accordance with some embodiments of the present disclosure.

At block 710, method 700 can include accessing a treatment plan for a patient. In some embodiments, accessing a treatment plan can include accessing a treatment plan for a patient comprising a series of sequential treatment stages, each treatment stage associated with a particular palatal expander in a series of palatal expanders. The treatment plan may include a palatal expansion treatment plan, an orthodontic treatment plan, and so on.

At block 720, processing logic receives one or more 2D images of an oral cavity of the patient during a treatment stage of the treatment plan. In some embodiments, processing logic may additionally or alternatively receive one or more 2D images of a face and/or smile of the patient. The 2D images may be generated by a patient during treatment without visiting a doctor. The 2D images may be generated, for example, using a mobile device such as a mobile phone, a tablet computer, a laptop computer, a digital camera, a webcam attached to a laptop or desktop computer, and so on. The 2D images may be uploaded to a server computing device of a virtual dental care system. In embodiments, the patient is guided (e.g., via an application running on the patient's computing device) to take particular images. The patient may be guided to open their mouth a particular amount, to position an image sensor at a particular angle relative to the patient's oral cavity, and so on. The patient may be guided to take images with or without a current palatal expander inserted onto their upper dental arch.

At block 730, processing logic processes the 2D image(s) to determine one or more observations. In some embodiments, determining one or more observations can include determining, via processing of the image data, one or more observations associated with the treatment plan. In embodiments, processing logic processes the one or more images using one or more trained machine learning models that perform segmentation of the images. The one or more trained machine learning models may perform semantic segmentation and/or instance segmentation in embodiments. An output of the segmentation process may include pixel-level masks for one or more instances of dental structures, such as teeth, palatine rugae, gingiva, a palatal expander, and so on. In the case of images of a patient face/smile, the machine learning model(s) may identify facial features, which may be used to measure facial width, nose breadth, facial asymmetry, and so on. In some instances, facial image(s) may be processed using a generative model that outputs one or more predicted post-treatment facial images. The predicted post-treatment facial images may be processed (e.g., using a machine learning model) to identify facial features and/or measure facial width, nose breadth, facial asymmetry, and so on.

One or more measurements may be performed of the segmented image data. The measurements may include, for example, a measurement of a distance (e.g., longitudinal distance) between left and right-most molars of the upper dental arch (e.g., an arch width of the upper dental arch), a distance (e.g., longitudinal distance) between two or more palatine rugae contours, one or more distances between palatine rugae contours and one or more features such as teeth, a distance between one or more tooth surfaces and one or more palatal expander surfaces, a distance between one or more facial features (e.g., points on a nose, points on a jaw, etc.) and/or other distances. In embodiments, distances are measured in terms of digital units of measurement, such as pixels. Images may be registered to a 3D model of the upper dental arch of the patient that includes known sizes for teeth and/or other dental structures. Based on the registration, a conversion factor may be determined for converting between the digital units of measurement and physical units of measurement (e.g., such as mm, inches, etc.). The determined distances (e.g., in physical units of measurement) may be compared to one or more thresholds, to target distances in a treatment plan, to initial distances measured at one or more prior points in time (e.g., at one or more prior treatment stages), etc. Based on the comparison(s), processing logic may determine one or more observations. For example, processing logic may determine that an arch width is below a threshold or has not reached a target arch width for a current stage of treatment. Alternatively, processing logic may determine that an arch width has reached a target arch width. Many other observations may also be made, as discussed above. Additionally, the segmented image data may be processed to measure angles of one or more teeth. Such angle measurements may be used to determine an amount of tipping of one or more teeth (e.g., molars) in a buccal or lingual direction. In embodiments, one or more measured angles may be compared to one or more angle thresholds to determine, for example, that one or more teeth (e.g., molars) have been tipped by an amount that is greater than an acceptable threshold, which may call for stopping palatal expansion treatment or modifying the palatal expansion treatment.

At block 740, processing logic can determine a level of progress of the treatment plan. In some embodiments, determining a level of progress can include determining, based on the one or more observations, a level of progress associated with the treatment plan. This may include determining whether the amount of palatal expansion (e.g., achieved arch width) is on track, is behind schedule, is ahead of schedule, etc.

At block 750, processing logic can provide a representation of progress of the treatment plan. In some embodiments, providing a representation of progress can include providing a representation of progress corresponding to the determined level of progress. The representation of progress may include a visual representation, which may include graphically showing a difference between a current dental arch width and a planned dental arch width. For example, processing logic may output the generated image showing the current arch width with a projected overlay of planned arch width. The representation of progress may additionally or alternatively include a numerical representation comprising one or more values that indicate the progress and/or a textual representation of the progress.

In some embodiments, at block 752 processing logic performs one or more actions based on the determined level of progress. The one or more actions may include adjusting an amount of time that a current palatal expander is to be worn. For example, if the arch width has reached a target arch width earlier than planned, the patient may be advanced to a next stage of treatment (and a next palatal expander or a widening of a current palatal expander) earlier than planned. In another example, if the arch width has failed to reach a target arch width within a planned treatment time, processing logic may determine to slow down treatment and prolong the amount of time that the patient wears the current palatal expander (and thus the amount of time that the patient remains in a current treatment stage). In some embodiments, the one or more actions include adding one or more new stages of treatment (e.g., which may include generating a 3D model of the patient's upper dental arch and/or of a palatal expander at one or more new stages, and using such 3D models to generate one or more new palatal expanders).

In one embodiment, at block 754, processing logic determines a recommendation based on the level of progress (e.g., a recommendation to advance to a next stage of treatment, to stay at a current stage of treatment, etc.). At block 756, processing logic sends the recommendation to a client device of a doctor and/or of a patient.

In one embodiment, at block 758 processing logic determines to advance the patient to a next treatment stage or to retain the patient at a current treatment stage.

FIG. 7B illustrates a flow diagram of an example method 760 for generating measurements used to monitor palatal expansion progress, in accordance with some embodiments of the present disclosure. Method 760 may be performed at block 730 of method 700 in some embodiments.

At block 761 of method 760, processing logic processes one or more received 2D images to identify features in the 2D image(s). This may include processing the 2D image(s) using one or more trained machine learning models to perform segmentation of the image(s) into one or more dental structures.

At block 762, processing logic determines measurements of one or more features (or distances between features) in the one or more 2D images in units of digital measurement. In some embodiments, the 2D image(s) may include one or more synthetic 2D images of a post-treatment condition of the patient's face, teeth and/or palate as output by a generative model. The measurements may be made between oral structures identified from the segmentation of block 761 in some embodiments. Examples measurements include measurements of a tooth angle (e.g., angle relative to a plane of a dental arch), arch width (e.g., distance between left and right-most molars on opposite sides of upper dental arch), distance between two front teeth (e.g., diastema measurement), distance between palatine rugae contours, distance between a palatine rugae contour and a tooth or other dental structure, facial width, nose breadth, and so on.

At block 764, processing logic converts the measurement(s) from the units for digital measurement into units for physical measurement. This may include at block 765 registering the one or more 2D images to at least one of an image or a 3D model comprising one or more teeth of the patient that have known physical measurements. At block 766, processing logic may then determine a conversion factor for converting between the units for digital measurement and the units for physical measurement based on a result of the measuring. In an example, the conversion factor may be determined by counting the number of pixels of a dental structure (e.g., a tooth) of the 2D image that corresponds to size of that dental structure in the 3D model after registration. Such a conversion factor may be an average conversion factor to be applied to multiple distances measured from the image. Alternatively, different conversion factors may be determined for different measurements. Conversion factors may be generated based on an average of conversions of pixel count to measurement at multiple different regions in the 2D image in some embodiments.

At block 767, processing logic may estimate an angle associated with a position of an image sensor used to capture the image(s) relative to the patient's upper dental arch. The angle may be a determined yaw angle in some embodiments.

At block 768, processing logic may compute a perspective correction factor based on the estimated angle. If the camera had a yaw angle relative to the patient's upper dental arch, this may cause teeth on one side of the image (e.g., right side) to appear larger than teeth on an opposite side of the image (e.g., left side). As a result, a correction factor computed for teeth at one side of the image may not be accurate for teeth at an opposite side of the image. In some embodiments, a perspective correction factor may be computed using basic trigonometric calculations. The perspective correction factor may result in a projection of a computed distance in an imaged plane (e.g., camera plane) onto a distance in a new plane (e.g., a front-facing plane of the patient's face).

At block 769, processing logic may modify the measurement based on applying the computed perspective correction factor to the computed measurement(s)

FIG. 7C illustrates a flow diagram of an example method 770 for determining palatal expansion progress based on measurements associated with a palatine rugae, in accordance with some embodiments of the present disclosure. Method 760 may be performed at block 730 of method 700 in some embodiments. At block 772 of method 770, processing logic determines a first distance between a feature (e.g., a tooth, a portion of a tooth, an identifiable point on the gingiva, a first palatine rugae contour or ridge, or any other biological reference points) and a palatine rugae contour or ridge (e.g., a second palatine rugae contour or ridge) in the one or more images. The feature may be any identifiable feature in a patient's mouth that is permanent or semi-permanent, and that can be identified in images taken at different times. The feature may be used as a reference point in embodiments. The first distance may be a distance between points on two adjacent palatine rugae contours or ridges in one example (e.g., where the feature is a particular palatine rugae contour or ridge). Additionally, or alternatively, processing logic may determine a first shape and/or size of the palatine rugae in the one or more 2D images.

At block 774, processing logic may determine a second distance between the feature and the palatine rugae in one or more previous 2D images. Additionally, or alternatively, processing logic may determine a second shape and/or size of the palatine rugae in the one or more previous 2D images.

At block 776, processing logic may compare the first distance to the second distance and/or the first shape to the second shape and/or the first size to the second size. Based on the comparison, processing logic may determine a difference between the first and second distances and/or between the first and second shape and/or between the first and second size. Based on the determined difference(s), processing logic may ultimately determine whether a change in position of one or more teeth (e.g., molars, premolars, etc.) is caused by palatal expansion (skeletal in nature) or tooth tipping (e.g., based on tooth movement). Thus, this method may enable a more accurate monitoring of the progress of a palatal expansion treatment by more directly measuring expansion across the palatine rugae. Additionally, the method may be used to determine an amount of tipping that may then be addressed (e.g., by an intervention such as modifying a treatment plan and/or designing/manufacturing a modified set of subsequent palatal expanders) or monitored.

FIG. 7D illustrates a flow diagram of an example method 780 for determining palatal expansion progress based on measurements associated with a palatine rugae, in accordance with some embodiments of the present disclosure. At block 782 of method 780, processing logic receives first image data of palatine rugae taken at a first time period during a first treatment stage or prior to treatment and second image data of the palatine rugae taken at a second time period during a second treatment stage. The first and second image data may include 2D intraoral images of the patient's upper palate (e.g., upper occlusal photos), and may show the patient's palatine rugae.

At block 784, processing logic may perform segmentation of the first image data and the second image data. Based on the segmentation, various teeth, palatine rugae features or ridges, gingiva and/or other oral objects or biological reference points may be identified. For each image, segmentation information comprising pixel-wise masks for each instance of an oral object (e.g., for each palatine rugae contour or ridge, each tooth, etc.) may be generated in embodiments. Additionally, or alternatively, the contours/ridges of the palatine rugae may be identified using edge detection, contour detection, color references, and/or other techniques, which may or may not involve application of machine learning models.

At block 786, processing logic compares palatine rugae features (e.g., shapes of the palatine rugae, lengths of palatine rugae ridges, distances between palatine rugae ridges, etc.) between the first image data and the second image data. In one embodiment, the palatine rugae of the first image data are aligned with the palatine rugae of the second image. This may include performing image registration between the first image and the second image. Multiple approaches can be used to align the rugae contours, including identifying the incisive papilla through object detection and using it as a reference point to align the rugae contours. Other approaches include computing/optimizing a transform (e.g., such as a homography transform, an affine transform or other transform) that best aligns the current and previous/initial rugae contours. The transformation can be found using matching points or by optimizing over a distance metric between the current rugae contours and the previous rugae contours. Different techniques that may be applied to perform image registration include scale-invariant transform (SIFT), speeded-up robust features (SURF), KAZE, AKAZE, binary robust independent elementary features (BRIEF), oriented FAST and rotated BRIEF (ORB), and binary robust invariant scalable key points (BRISK) algorithms.

Once the current rugae contours have been aligned to the initial or previous set of contours, the contour distance can be found (e.g., distance between palatine rugae contours). Contour distance will increase with the amount of palatal (skeletal) expansion.

At block 788, processing logic determines a difference between the palatine rugae of the first image (e.g., past palatine rugae) and the palatine rugae of the second image (e.g., current palatine rugae). A variety of distance metrics can be used, both for assessing the change in rugae and for computing an optimal transform. In one embodiment, a Hausdorff distance or a modified (one-way) Hausdorff distance can be found. Other options include identifying matching points (e.g., using biological reference points or image processing techniques such as SIFT, SURF, or ORB) and computing a mean distance between the matching points.

Skeletal expansion is usually accompanied by changes to the palatine rugae. By monitoring the rugae, processing logic can differentiate between palatal widening and dental expansion (i.e., determine if the palate is widening or if the dentition is moving/tilting). In some embodiments, processing logic determines whether palatal expansion treatment has resulted in an expanded palate (as planned) or in tipping of one or more teeth based at least in part on processing information about palatine rugae of the patient (e.g., as described above with reference to FIGS. 7C-D). If the spacing between a patient's teeth is expanding but the palatine rugae are not changing, this indicates that the expansion of the spacing between the patient's teeth is caused by tooth tipping. Responsive to identifying tooth tipping, processing logic may update a treatment plan to include one or more treatment stages that apply palatal expanders that are shaped to offer counter tipping forces.

To control tooth (e.g., crown) tipping, one or more palatal expander can be designed to add “anti-tipping” torque to one or more teeth. In one embodiment, one or more attachments to teeth may be used to apply anti-tipping forces. One way to add “anti-tipping” torque is by design the shape of a palatal expander so that it contacts with the palate surface and can push the palate near the midline suture. For example, the space between the palate and expander may be adjusted to apply force at a lateral contact point on the lingual side of the palate. In embodiments, anti-tipping features may be designed into palatal expanders for one or more stages of treatment as described in U.S. application Ser. No. 18/158,451, filed Jan. 23, 2023, which is incorporated by reference herein in its entirety. In embodiments, responsive to identifying tooth tipping, a treatment plan is updated by modifying the design of palatal expanders for one or more treatment stages by adding anti-tipping features to the palatal expanders.

FIG. 7E illustrates a flow diagram of an example method 790 for determining palatal expansion progress based on measurements associated with a dental arch width, in accordance with some embodiments of the present disclosure. Method 790 may be performed at block 730 of method 700 in some embodiments. At block 792 of method 790, processing logic measures an arch width of an upper dental arch in units of digital measurement from an image of an open mouth view of an upper palate for a patient. This may include performing segmentation, and then measurement of segmented objects, as described above. At block 794, processing logic converts the arch from units of digital measurement to units of physical measurement as previously discussed. At block 796, processing logic compares the arch width in units of physical measurement to a target arch width of a treatment plan. Based on a result of the comparison, processing logic may determine whether palatal expansion is on track, proceeding faster than planned, or proceeding slower than planned.

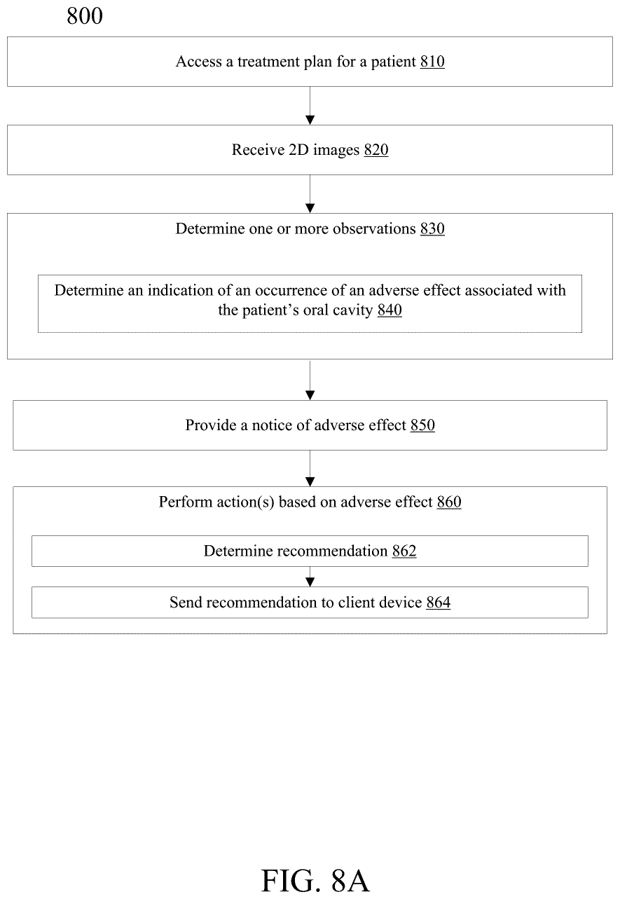

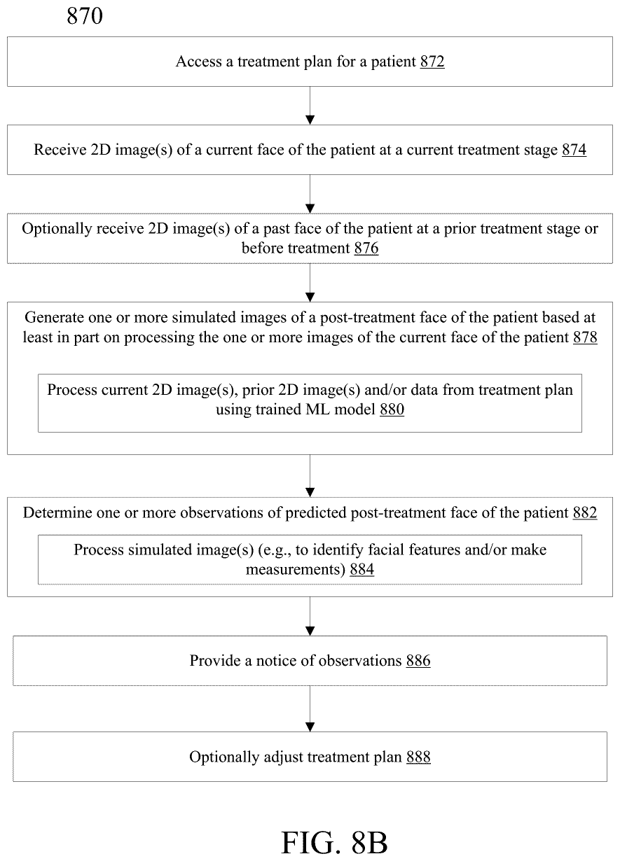

FIG. 8A illustrates a flow diagram of an example method 800 for monitoring palatal expansion, in accordance with some embodiments of the present disclosure. At block 810, method 800 can include accessing a treatment plan. In some embodiments, accessing a treatment plan can include accessing a treatment plan for a patient comprising a series of sequential treatment stages, each treatment stage associated with a particular palatal expander in a series of palatal expanders.

At block 820, processing logic receives one or more 2D images of a patient's oral cavity. In some embodiments, the 2D images are of the patient's dentition and/or upper palate while a palatal expander is worn. Alternatively, the 2D images may be of the patient's dentition and/or upper palate without the palatal expander.

At block 830, processing logic determines one or more observations from the 2D images, as described above. In some embodiments, extracting one or more observations can include extracting, via processing of the 2D images and/or sensor data, one or more observations associated with the treatment plan. In some embodiments, determining the one or more observations includes at block 840 determining an indication of an occurrence of an adverse effect associated with the patient's oral cavity. In some embodiments, determining one or more observations are performed based on performing segmentation of the image(s) and then measuring one or more values associated with segmented dental structures. For example, processing logic may measure an amount of buccal or lingual tipping of one or more molars, the distance between front teeth (e.g., for a diastema measurement), etc. Additionally, or alternatively, one or more 2D images may be processed using traditional image processing and/or one or more trained machine learning models trained to identify the presence or absence of one or more adverse effects/events in images. For example, a machine learning model may be trained to identify the presence of tissue damage, of impressions caused by wearing a palatal expander, of an excessively worn palatal expander, and so on. Such a trained machine learning model may process an input image to determine, for example, whether a patient has experienced tissue damage as a result of wearing a palatal expander. Additionally, or alternatively, the machine learning model may output a bounding shape around an identified tissue damage, may output a mask indicating pixels of tissue damage, and so on.

In one example, at least one 2D image of the oral cavity of the patient was taken while a current palatal expander in a series of palatal expanders was worn and shows the current palatal expander. In such an embodiment, processing logic may determine a level of wear of the current palatal expander based on processing of the at least one 2D image. In one embodiment, determining the level of wear of the current palatal expander comprises processing the at least one 2D image using a trained machine learning model that outputs the level of wear of the current palatal expander. In one embodiment, processing logic compares the determined level of wear of the current palatal expander to a wear threshold. The determination that the wear exceeds the wear threshold may be an identified adverse effect.

At block 850, processing logic provides a notification of one or more identified adverse effects. The notification may be a textual notification, an audio notification, and/or a graphical/visual notification. In some embodiments, the notification includes an image of the adverse effect, such as an image of a tipped molar with a measured tipped angle, an image with an outline around tissue damage, and so on.

In some embodiments, at block 860 processing logic performs one or more actions based on the determined adverse effect. The one or more actions may include adjusting an amount of time that a current palatal expander is to be worn, stopping treatment, inviting a patient to schedule an in-person visit to the doctor office, redesigning one or more palatal expanders, instructing a patient to advance to a next palatal expander in a treatment plan, replacing a current palatal expander with a new palatal expander, and so on.

In one embodiment, at block 862, processing logic determines a recommendation based on the detected adverse effect. At block 864, processing logic sends the recommendation to a client device of a doctor and/or of a patient.

FIG. 8B illustrates a flow diagram of an example method 870 for predicting a post-treatment face of a patient associated with palatal expansion and/or orthodontic treatment. At block 872, method 870 can include accessing a treatment plan. The treatment plan may be a treatment plan for palatal expansion treatment and/or for orthodontic treatment in embodiments. In some embodiments, accessing a treatment plan can include accessing a treatment plan for a patient comprising a series of sequential treatment stages, each treatment stage associated with a particular palatal expander in a series of palatal expanders and/or a particular orthodontic aligner in a series of orthodontic aligners.

At block 874, processing logic receives one or more 2D images of a current face of the patient. Processing logic may also receive one or more 2D images of the patient's oral cavity in embodiments, which may be processed using the techniques set forth herein in parallel to the processing of the facial images.

At block 876, processing logic optionally receives one or more 2D images of a past face of the patient at a prior treatment stage and/or prior to treatment. The 2D images of the past face may have been captured prior to treatment and/or at one or more prior treatment stages, and may have been stored in a patient record in embodiments.

At block 878, processing logic generates one or more simulated images of a post-treatment face of the patient based at least in part on processing the one or more images of the current face of the patient. In some embodiments, at block 880 processing logic processes the current 2D image(s), the prior 2D image(s), and/or data from a treatment plan using a trained machine learning model (e.g., a generative model). In some embodiments, a stage of treatment associated with each of the 2D images (e.g., a current stage of treatment associated with the current facial image) is input into the machine learning model, and a total number of stages of treatment is input into the machine learning model. The machine learning model may output a synthetically generated post-treatment image of the patient's face.

At block 882, processing logic determines one or more observations of the predicted post-treatment face of the patient. The one or more observations may include, for example, a face width measurement, a nasal breadth measurement and/or a facial asymmetry measurement. In one embodiment, at block 884 processing logic processes the simulated image(s) to identify facial features and/or determine one or more measurements. In one embodiment, the simulated images are processed using traditional image processing techniques. In one embodiment, the simulated images are processed using a machine learning model. In one embodiment, the simulated images are processed using a combination of traditional image processing techniques and a machine learning model. In an example, a machine learning model may process the simulated image(s) to identify one or more facial features in the simulated image(s). One or more measurements may then be performed using the facial features. The one or more measurements may be performed by the machine learning model and/or using traditional image processing techniques (e.g., identified features may be selected on the image and a measurement may be made of a distance between the identified features). Examples of such features include points on a nose, points corresponding to cheek bones, points corresponding to jaw bones, and so on.

At block 886, processing logic may provide a notification of the one or more observations to the doctor and/or to the patient (e.g., to a doctor device and/or a patient device, which may output the notification via a display of the doctor device and/or patient device). In some embodiments, the post-treatment image(s) may be provided to the doctor and/or patient. In some embodiments, the post-treatment image(s) are annotated with the observations (e.g., marked with measurements). In some embodiments, a video showing a progression of the patient's facial appearance from pre-treatment to post-treatment is generated, which may be sent to a doctor device and/or patient device. The patient, on reviewing the post-treatment image(s), the observation(s), and/or the video, may determine that the predicted post-treatment facial appearance is not acceptable to the patient. In some embodiments, processing logic compares one or more generated measurements (e.g., of facial width, nose breadth, etc.) to one or more thresholds. If the one or more measurements exceed the one or more thresholds, then a notification may be output.

At block 888, processing logic may determine whether to adjust the treatment plan. The treatment plan may be optionally adjusted in view of the determined observations. For example, if the patient determined that the predicted post-treatment facial appearance for the patient is not acceptable, then treatment may be shortened (e.g., by reducing a number of treatment stages and/or a planned arch width expansion) or stopped completely.

In some cases, the palatal expansion may introduce a facial asymmetry (e.g., due to a primary suture of the upper palate being at an angle, uneven splitting of one or more sutures, etc.). Such an asymmetry may be one of the observations of the predicted post-treatment image of the patient's face. If such an asymmetry is predicted, processing logic may update the palatal expansion treatment plan by adjusting one or more palatal expanders in a sequence of palatal expanders to adjust one or more forces applied by the one or more palatal expanders to a palate of the patient to counter the predicted facial asymmetry. Adjusting the one or more palatal expanders may include modifying at least one of a rigidity, a thickness, or a material for one or more regions of the one or more palatal expanders. The rigidity, thickness and/or material at the region(s) may be selected to induce uneven forces on the patient's palate. The uneven forces may be planned to counteract the developing and/or predicted asymmetry. In some instances, a predicted asymmetry may be counteracted by performing surgery to open up one or more sutures of the patient's palate. For example, a suture on one side of the patient's palate may have opened (e.g., either via surgery or naturally as a result of the palatal expansion), but the corresponding suture on the opposite side of the mouth may not have opened. Opening up the suture surgically may prevent or ameliorate the predicted asymmetry. Accordingly, a recommendation to perform surgery to loosen one or more sutures of a palate of the patient to counteract the predicted facial asymmetry may be output to a doctor.

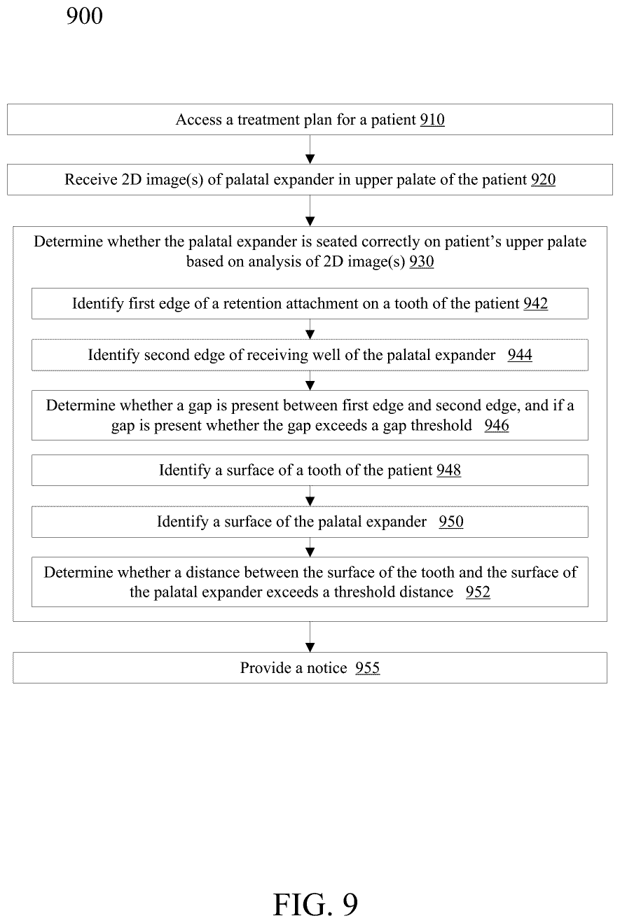

FIG. 9 illustrates a flow diagram of an example method 900 for determining whether a palatal expander is worn correctly, in accordance with some embodiments of the present disclosure. At block 910, method 900 can include accessing a treatment plan. In some embodiments, accessing a treatment plan can include accessing a treatment plan for a patient comprising a series of sequential treatment stages, each treatment stage associated with a particular palatal expander in a series of palatal expanders.

At block 920, processing logic receives one or more 2D images of a palatal expander in an upper palate of a patient (i.e., of the patient wearing the palatal expander).

At block 930, processing logic determines one or more observations from the 2D images, as described above. In some embodiments, determining the one or more observations includes determining whether the palatal expander is seated correctly on the patient's upper teeth based on analysis of the 2D images.

In one embodiment, processing logic performing segmentation of the image(s) and then measures one or more values associated with segmented dental structures. For example, processing logic may identify a first edge of a retention attachment on a tooth of the patient at block 942, and may identify a second edge of a receiving well of the palatal expander configured to engage with the retention attachment at block 944. At block 946, processing logic may determine whether a gap is present between the first edge and the second edge, and if a gap is present, whether the gap exceeds a gap threshold. This may include measuring a distance between the first edge and the second edge in units of digital measurement and converting the distance to units of physical measurement as described above. The distance in units of physical measurement may be compared to a gap threshold. If the gap exceeds a gap threshold, then processing logic may determine that the palatal expander is not properly seated in the patient's upper dental arch, or that the palatal expander does not fit the patient for some reason.

In another example, at block 948 processing logic identifies a surface of a tooth of the patient, and at block 950 processing logic identifies a surface of the palatal expander (e.g., that is to engage with the surface of the tooth). At block 952, processing logic may then measure a distance between the surface of the tooth and the surface of the palatal expander (e.g., in units of digital measurement, and then converted to units of physical measurement), and determine whether the distance exceeds a distance threshold. If the distance exceeds a distance threshold, then processing logic may determine that the palatal expander is not properly seated in the patient's upper dental arch, or that the palatal expander does not fit the patient for some reason.

At block 955, processing logic provides a notification of the one or more observations (e.g., that the palatal expander is not properly seated in the patient's upper dental arch, or that the palatal expander does not fit). If a determination was made that the palatal expander is not properly seated, then the notification may include instructions to the patient on how to properly seat the palatal expander in their upper dental arch (e.g., by outputting a text prompt, image and/or video to a computing device of the patient). In some cases, processing logic may output an instruction for the user to use a device to aid with properly seating the palatal expander in their upper dental arch (e.g., aligner “chewies” or some other suitable device).

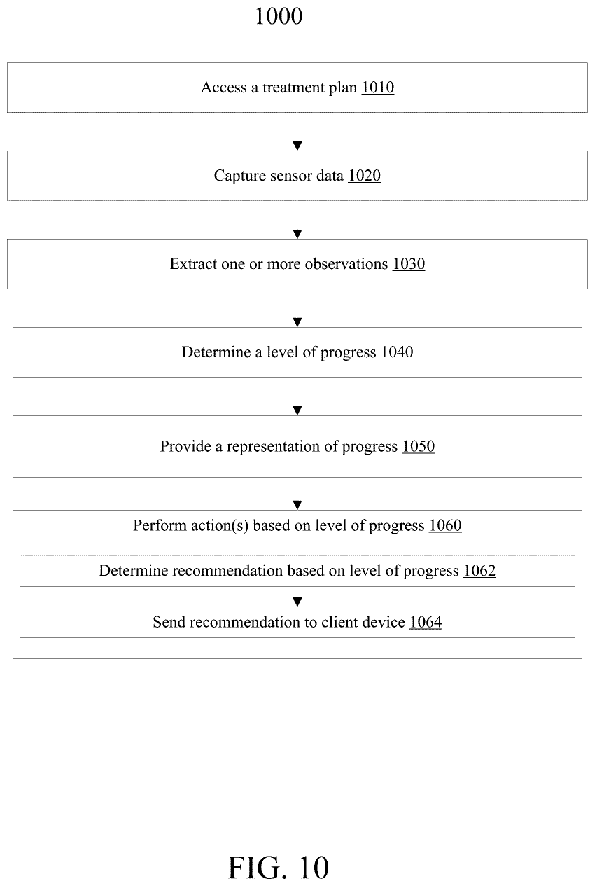

FIG. 10 illustrates a flow diagram of an example method 1000 for monitoring a palatal expansion based on sensor measurements, in accordance with some embodiments of the present disclosure. At block 1010, method 1000 can include accessing a treatment plan for a patient. In some embodiments, accessing a treatment plan can include accessing a treatment plan for a patient comprising a series of sequential treatment stages, each treatment stage associated with a particular palatal expander in a series of palatal expanders. The treatment plan may include a palatal expansion treatment plan, an orthodontic treatment plan, and so on.

At block 1020, processing logic receives one or more sensor measurements generated by sensors of a palatal expander worn by a patient during a treatment stage of a treatment plan. The sensor measurements may include transverse force measurements indicating a transverse force on the palatal expander indicative of an amount of force that the palatal expander is exerting on the patient's dental arch to expand the patient's palate. A transverse force that is below a lower threshold may not cause a clinical change in the patient's arch width. On the other hand, a transverse force that is above an upper threshold may cause the patient's molars to become tipped by an unacceptable amount, may cause undue pain to the patient, or otherwise result in complications. Forces may gradually increase as progressively wider palatal expanders are used (e.g., as treatment progresses through multiple stages). Such an increase in force is anticipated. However, if the force is determined to exceed a threshold, this may indicate that the palate is more resistive to expansion than anticipated. Under such situations, it may be recommended to slow down further expansion (e.g., retain a current stage of palatal expansion) until the measured transverse force declines.

Sensor measurements may also include an amount of force (e.g., bending force, vertical force, rotational force, etc.) experienced by the palatal expander during use. Such force may be measured, for example, during insertion of the palatal expander onto the patient's upper palate and during removal of the palatal expander from the upper palate. If the force is above a threshold, this may damage the palatal expander, and may be an indication that the palatal expander is not being inserted/removed with a correct technique.

Sensor measurements may also include a rotational force (e.g., torque or rotational moment) exerted on one or more palatal region(s) of the palatal expander. Such forces may indicate an amount of rotational forces exerted on one side (e.g., right side) of the patient's palate relative to an opposite side (e.g., left side) of the patient's palate in embodiments (e.g., an amount of torque applied between a first palatal region and a second palatal region).

Sensor measurements may also include a rotational force (e.g., torque or rotational moment) exerted on one or more tooth region (and thus on one or more associated teeth).

Sensor measurements may additionally include measurements of bite force at one or more tooth receiving regions of the palatal expander. For example, the one or more integrated sensors may comprise a plurality of force sensors or a plurality of pressure sensors disposed at a plurality of regions of the palatal expander and configured to measure bite force at the plurality of regions.

Sensor measurements may additionally include a contact measurement indicating whether the palatal expander is touching the upper palate and/or a pressure measurement indicating an amount of pressure between the upper palate and the palatal expander. Such contact and/or pressure may indicate that the palatal expander has a height or depth that is too large, and that causes the palatal expander to impinge on the tissue of the patient's upper palate, which can cause pain and/or injury to the patient.

Many other sensor measurements may also be collected, as described above.

At block 1030, processing logic processes the sensor data to determine one or more observations. In some embodiments, determining one or more observations can include determining, via processing of the image data, one or more observations associated with the treatment plan. In some embodiments, processing logic processes the sensor data using one or more trained machine learning models that output one or more observations. Alternatively, or additionally, sensor data may be compared against thresholds and/or other criteria to determine one or more observations.

In some embodiments, a rotational force (e.g., torque or rotational moment) exerted on one or more tooth regions (and thus on one or more associated teeth) is compared to one or more torque thresholds. Such forces may indicate a tipping force applied to one or more teeth, and may be used to predict whether a tooth is likely to tip during treatment. If a rotational force exerted on one or more teeth exceeds a threshold, then a notification may be generated. In some embodiments, a recommendation to adjust a treatment plan may be generated. For example, one or more palatal expanders in a series of palatal expanders may be modified to reduce the rotational force exerted on the one or more teeth. In embodiments, processing logic may determine one or more adjustments to one or more palatal expanders that will reduce the rotational forces on the teeth, and may recommend such adjustments to a doctor.

In some embodiments, one or more actions may be performed based on a determined bite force as measured by one or more force/pressure sensors configured to measure bite force/pressure. In one embodiment, processing logic counts a number of times that the patient bites the palatal expander based on measurements of one or more force sensors or the one or more pressure sensors (e.g., at block 1030). Processing logic may determine whether the number of times that the patient bites the palatal expander exceeds a bite count threshold. If the bite count exceeds a bite count threshold, this may indicate that a structural integrity of the palatal expander has been compromised. Processing logic may then output a notification notification to replace the palatal expander responsive to determining that the number of times that the patient bites the palatal expander exceeds the bite count threshold.

In some embodiments, processing logic determines whether a measured bite force exceeds a bite force threshold. Excessive bite forces may compromise a structural integrity of the palatal expander. Accordingly, if the bite force threshold is exceeded, processing logic may output a notification to replace the palatal expander.

In some embodiments, processing logic analyzes the measured bite forces over time to determine whether a patient suffers from bruxism and/or to determine when the patient suffers from bruxism (e.g., to determine whether the patient grinds or clenches their jaw during sleep). If bruxism is identified, processing logic may output a notification of the identified bruxism and/or a timing of the identified bruxism. Additionally, or alternatively, one or more palatal expanders may be modified in view of identified bruxism (e.g., by increasing a thickness of the palatal expanders to make them more resistant to the bruxism).

If the sensor data includes bite force data from a plurality of locations of the palatal expander, such data may be used to assess a patient bite in embodiments. For example, processing logic may measure bite force or bite pressure at a plurality of regions of the palatal expander using the plurality of force sensors or the plurality of pressure sensors. Processing logic may then perform a bite analysis based on the measured bite force or bite pressure at the plurality of regions of the palatal expander. The bite analysis may indicate one or more bite contact points of the patient (e.g., by identifying points that experience maximal bite force). Such bite contact points may be compared against optimal bite contact points determined for the patient (e.g., based on human physiology). Processing logic may determine one or more modifications to one or more palatal expanders in a sequence of palatal expanders to be worn by the patient, wherein the one or more modifications improve bite contact points for the patient while the patient wears the one or more palatal expanders. The one or more modifications comprise changing a thickness of one or more occlusal regions of the one or more palatal expanders to change the bite contact points.

In one embodiment, at block 1040 processing logic determines a level of progress of palatal expansion based on the one or more observations. This may include determining whether the amount of palatal expansion (e.g., achieved arch width) is on track, is behind schedule, is ahead of schedule, etc. For example, processing logic may determine that palatal expansion is slower than anticipated based on the transverse force exceeding an upper force threshold, or may determine that expansion is faster than anticipated based on the transverse force being below a lower force threshold.

At block 1050, processing logic can provide a representation of progress of the treatment plan. In some embodiments, providing a representation of progress can include providing a representation of progress corresponding to the determined level of progress. The representation of progress may include a visual representation, which may include graphically showing a difference between a current dental arch width and a planned dental arch width. For example, processing logic may output the generated image showing the current arch width with a projected overlay of planned arch width.

In some embodiments, at block 1060 processing logic may perform one or more actions based on the determined level of progress. The one or more actions may include adjusting an amount of time that a current palatal expander is to be worn. For example, if the transverse force is below a lower threshold, the patient may be advanced to a next stage of treatment (and a next palatal expander or a widening of a current palatal expander) earlier than planned. In another example, if the force is above an upper force threshold, processing logic may determine to slow down treatment an prolong the amount of time that the patient wears the current palatal expander (and thus the amount of time that the patient remains in a current treatment stage). In some embodiments, the one or more actions include adding one or more new stages of treatment (e.g., which may include generating a 3D model of the patient's upper dental arch and/or of a palatal expander at one or more new stages, and using such 3D models to generate one or more new palatal expanders).

In one embodiment, at block 1062, processing logic determines a recommendation based on the level of progress (e.g., a recommendation to advance to a next stage of treatment, to stay at a current stage of treatment, etc.). At block 1064, processing logic sends the recommendation to a client device of a doctor and/or of a patient.

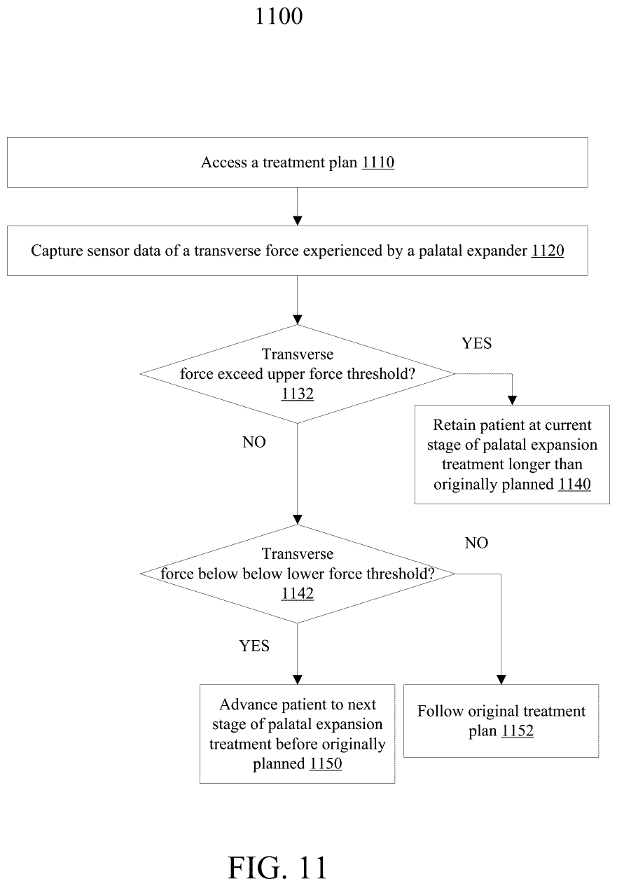

FIG. 11 illustrates a flow diagram of an example method 1100 for determining how to adjust a treatment plan based on sensor data associated with transverse force on a palatal expander, in accordance with some embodiments of the present disclosure. At block 1010, method 1000 can include accessing a treatment plan for a patient. In some embodiments, accessing a treatment plan can include accessing a treatment plan for a patient comprising a series of sequential treatment stages, each treatment stage associated with a particular palatal expander in a series of palatal expanders. The treatment plan may include a palatal expansion treatment plan, an orthodontic treatment plan, and so on. At block 1020, processing logic receives one or more sensor measurements generated by sensors of a palatal expander worn by a patient during a treatment stage of a treatment plan, which capture sensor data of a transverse force experienced by the palatal expander.

At block 1132, processing logic determines whether the force exceeds an upper force threshold. If so, the method proceeds to block 1140 and processing logic retains the patient at a current stage of palatal expansion treatment longer than originally planned. Otherwise, the method proceeds to block 1142.

At block 1142, processing logic determines whether the force is below a lower force threshold. If so, the method proceeds to block 1150 and processing logic advances the patient to a new stage of palatal expansion treatment before originally planned. Otherwise, the method proceeds to block 1152, and the original treatment plan is followed without adjustment.

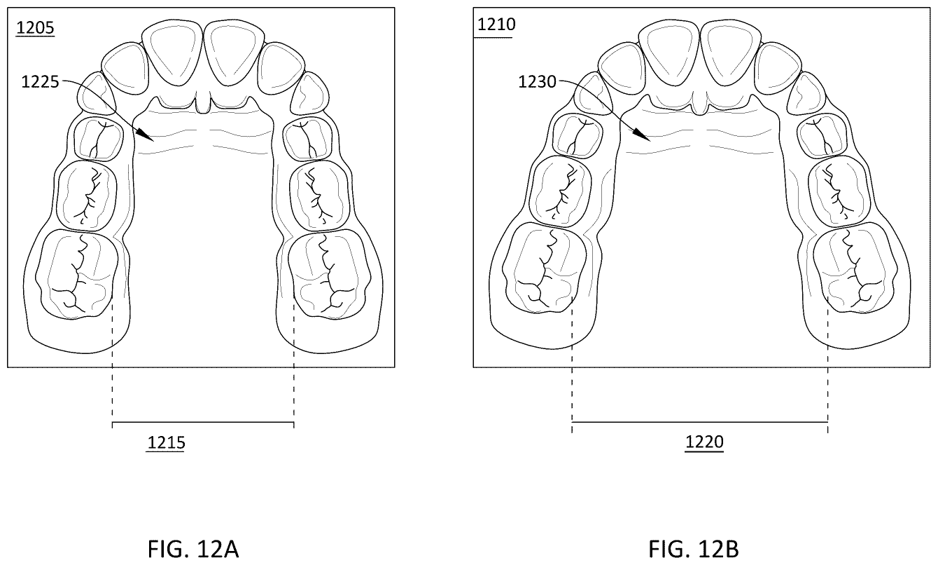

FIG. 12A illustrates an image 1205 of an upper dental arch of a patient prior to palatal expansion treatment, according to certain embodiments. As shown, the palatine rugae 1225 in image 1205 have a first shape and the arch width 1215 has a first value.

FIG. 12B illustrates an image 1210 of an upper dental arch of the patient captured at a later time during a stage of palatal expansion treatment, according to certain embodiments. As shown, the palatine rugae 1230 in image 1210 have a second shape and the arch width 1220 has a second value.

Processing logic may process the image 1205 and image 1210 to segment the images into individual teeth, and measure the arch widths for the two images and then compare the arch widths to determine a level of progress of palatal expansion. Additionally, or alternatively, the measured arch width 1220 may be compared to an arch width of a treatment plan or a current stage of treatment. Additionally, the two images may be registered to one another, and shapes and/or positions of the palatine rugae 1225 may be compared to the shapes and/or positions of the palatine rugae 1230 to determine a level of progression of the palatal expansion, determine whether arch width expansion is attributable to skeletal expansion or tooth movement, and so on.

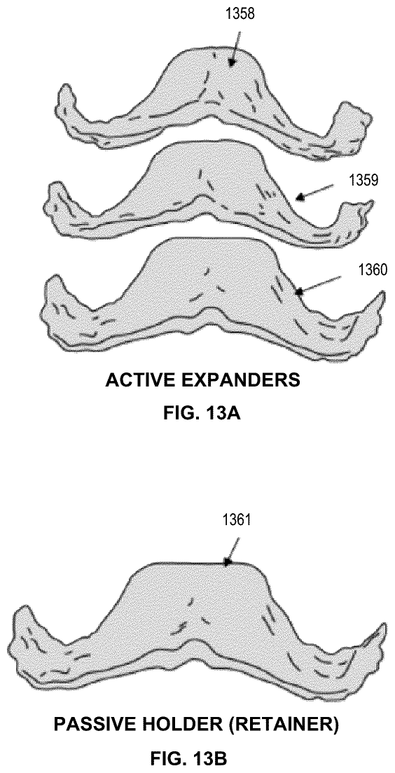

FIG. 13A shows an example of a series of palatal expanders that get progressively broader. For example, an initial upper palatal expander 1358 can have narrower palatal region than the intermediate palatal expander 1359 and a final palatal expander 1360.

FIG. 13B illustrates an example of a passive holder (e.g., retainer) 1361 that can be worn after the series has completed expanding the patient's palate. In this example, the palatal expander retainer 1361 is similar or identical to the last of the palatal expanders in the sequence, although it can have a different configuration.

As mentioned above, a palatal expander as described herein can be one of a series of palatal expanders (incremental palatal expanders) that can be used to expand a subject's palate from an initial size/shape toward a target size/shape. For example, the methods and improvements described herein can be incorporated into a palatal expander or series of palatal expander as described, for example, in US20190314119A1, herein incorporated by reference in its entirety. A series of palatal expanders can be configured to expand the patient's palate by a predetermined distance (e.g., the distance between the molar regions of one expander can differ from the distance between the molar regions of the prior expander by not more than 2 mm, by between 0.1 and 2 mm, by between 0.25 and 1 mm, etc.) and/or by a predetermined force (e.g., limiting the force applied to less than 180 Newtons (N), to between 8-200 N, between 8-90 N, between 8-80 N, between 8-70 N, between 8-60 N, between 8-50 N, between 8-40 N, between 8-30 N, between 30-60 N, between 30-70 N, between 40-60 N, between 40-70 N, between 60-200 N, between 70-180 N, between 70-160 N, etc., including any range there between).

The palatal region can be between about 1-5 mm thick (e.g., between 1.5 to 3 mm, between 2 and 2.5 mm thick, etc.). The occlusal side can have a thickness of between about 0.5-2 mm (e.g., between 0.5 to 1.75 mm, between 0.75 to 1.7 mm, etc.). The buccal side can have a thickness of between about 0.25-1 mm (e.g., between 0.35 and 0.85 mm, between about 0.4 and 0.8 mm, etc.).

The dental devices described herein can include any of a number of features to facilitate the expansion process, improve patient comfort, and/or aid in insertion/retention of the dental devices in the patient's dentition. Examples of some features of dental devices are described in U.S. Patent Application Publication No. 2018/0153648A1, filed on Dec. 4, 2017, which is incorporated herein by reference in its entirety. For example, any of the dental devices described herein can include any number attachment features that are configured to couple with corresponding attachments bonded to the patient's teeth. The dental devices can have regions of varying thickness. In any of the dental devices described herein can have varied thicknesses. For example, the thickness of a palatal region can be thicker or thinner than the thickness of tooth engagement regions. The palatal region of any of the palatal expanders can include one or more cut-out regions, which can enhance comfort and/or prevent problems with speech.

FIG. 14 illustrates an example system architecture 1400 capable of supporting a holistic monitor for monitoring a palatal expansion treatment plan, in accordance with one embodiment of the present disclosure.

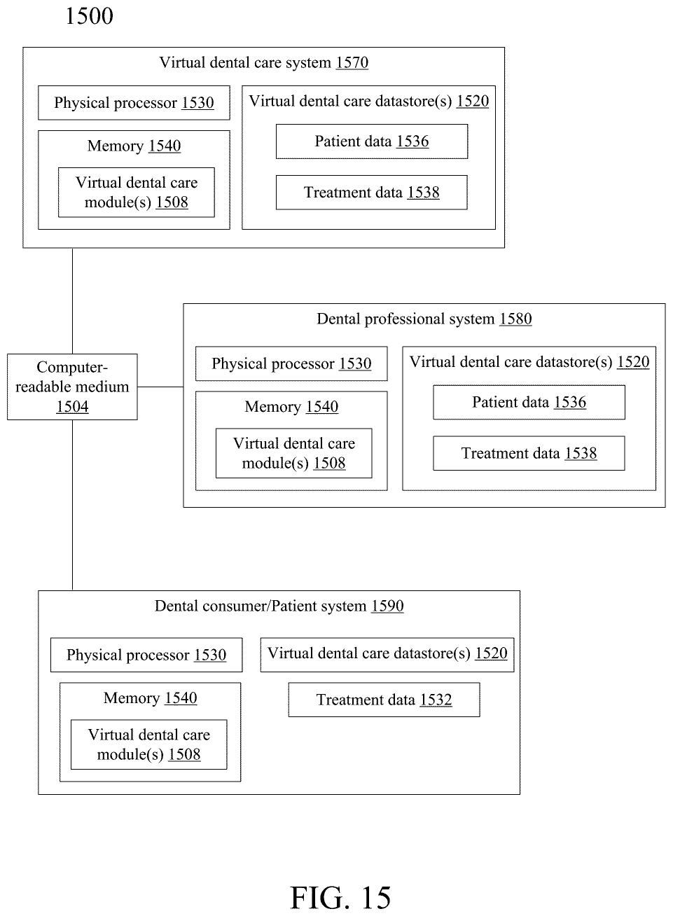

The treatment system architecture 1400 can correspond to system 1500 of FIG. 15, in embodiments. For example, system architecture 1500 can include a dental consumer/patient system 1590, a dental professional system 1580 and a virtual dental care system 1570. In embodiments, virtual dental care system 1570 includes a treatment plan coordination platform 1420, a holistic monitor 1450 and/or a storage platform 1440. In embodiments, dental consumer/patient system 1590 includes a client device 1430B and/or one or more connected palatal expander device(s) 1430C. In embodiments, dental professional system 1580 includes a client device 1430A. In some embodiments, any of the treatment plan coordination platform 1420, the client device(s) 1430A-C, the storage platform 1440, and/or the holistic monitor 1450, can include, can be, or can otherwise be connected to one or more computing devices (such as a rackmount server, a router computer, a server computer, a personal computer, a mainframe computer, a laptop computer, a tablet computer, a desktop computer, etc.), one or more storage devices (e.g., hard disks, memories, databases), networks, software components, and/or hardware components capable of connecting to system 1400. In embodiments a platform can support any number of discrete software or hardware, or combination of such, portions, which can be referred to as modules.

In some embodiments, network 1401 can connect the various platforms and/or devices, which can include a public network (e.g., the Internet), a private network (e.g., a local area network (LAN) or wide area network (WAN)), a wired network (e.g., Ethernet network), a wireless network (e.g., an 802.11 network or a Wi-Fi network), a cellular network (e.g., a Long Term Evolution (LTE) network), routers, hubs, switches, server computers, and/or a combination thereof.

In some embodiments, the treatment plan coordination platform 1420 can facilitate or host services for coordinating HCP-patient communications relating to an on-going treatment plan for a patient. In embodiments, the treatment plan coordination platform 1420 can host, leverage, and/or include several modules for supporting such system functionalities. For instance, in embodiments, platform 1420 can support and/or integrate a control module (not shown in FIG. 14), for performing overall control of modules and devices associated with the platform, and a user-interface (UI) control module (not shown in FIG. 14), for performing generation, and other processes associated with a UI that will be presented through associated client devices. Platform 1420 can support a data processing module (not shown in FIG. 14), that can gather and manage data from storage and modules (such as patient data and/or plan data gathered from storage device 1444 and/or storage platform 1440). Platform 1420 can also process, transmit, and/or receive incoming and outgoing data from client device 1430A and/or client device 1430B. Such modules can work collaboratively, and communicate internally or externally (e.g., to further systems and/or through APIs), to facilitate virtual meeting or communication capabilities for users across a range of client devices. Each module can include hardware, firmware, and/or software configured to provide a described functionality.

In some embodiments, platform 1420 (or an integrated control module) can orchestrate the overall functioning of the treatment coordination platform 1420. In some cases, platform 1420 can include algorithms and processes to direct the setup, data transfer, and processing for providing and receiving data associated with a treatment plan from connected devices (e.g., the client device 1430A-C). For example, when a user initiates engagement with the treatment plan coordination system 1400, the platform 1420 can initiate and manage the associated processes, including allocating resources, determining routing pathways for data and data streams, managing permissions, and so forth to interact with client devices to establish and maintain reliable connections and data transfer.

Platform 1420 can include a UI controller, and can perform user-display functionalities of the system such as generating, modifying, and monitoring the individual UI(s) and associated components that are presented to users of the platform 1420 through a client device. For example, a UI control module can generate the UI(s) (e.g., UIs 1434A-B of client devices 1430A-B) that users interact with while engaging with the treatment coordination system.

A UI can include many interactive (and/or non-interactive) visual elements for display to a user. Such visual elements can occupy space within a UI and can be visual elements such as windows displaying video streams, windows displaying images, chat panels, file sharing options, participant lists, and/or control buttons for controlling functions such as client application navigation, file upload and transfer, controlling communications functions such as muting audio, disabling video, screen sharing, etc. The UI control module can work to generate such a UI, including generating, monitoring, and updating the spatial arrangement and presentation of such visual elements, as well as working to maintain functions and manage user interactions, together with the platform 1420. Additionally, the UI control module can adapt a user-interface based on the capabilities of client devices. In such a way the UI control module can provide a fluid and responsive interactive experience for users of the treatment coordination platform.

In some embodiments, a data processing module can be responsible for storage and management of data. This can include gathering and directing data from client devices. In embodiments, the data processing module can communicate and store data, including to and/or from storage platforms and storage devices (e.g., such as storage device 1444), etc. For instance, once an initial treatment plan (e.g., initial treatment plan 1460) has been established, platform 1420 can perform tasks such as gathering and directing such data to the storage platform 1440, and/or to client devices 1430A-B.

In embodiments, data that is transmitted, managed, and/or manipulated by the system can include any kind of data associated with a treatment plan, including (e.g., treatment plan schedules, dates, times, etc.), patient data (e.g., such as images, values, sensor data, etc.), and so on.

In embodiments, the system 1400 can leverage a holistic monitor 1450 for performing processes associated with data collected by the client devices. In embodiments, the holistic monitor 1450 can include a dataset generator 1456 and an analysis module 1458. Holistic monitor 1450 can intake collected data 1462 (e.g., collected data from a patient's client device and/or from sensors of a palatal expander) and process such data to generate observations 1464, or progress indicators, extracted from the collected data 1462, and generate responses 1466. In a non-limiting example, holistic monitor 1450 can intake collected data 1462 that can include image data of a patient's oral cavity and/or sensor data from sensors of one or more palatal expanders. Holistic monitor 1450 can then extract observations 1464 from the image data and/or sensor data, such as an observation that a diastema within teeth of the patient has formed, that a force buildup is too high, that a palatal expander is being inserted and/or removed incorrectly, and so on. The holistic monitor 1450 can intake this data and effect an appropriate response 1466 (e.g., such as generate a notification for a patient, or HCP). In embodiments, responses 1466 generated by the holistic monitor 1450 can include updates to the initial treatment plan 1460, notifications sent to any of the client devices or platforms or modules associated with the system, and/or store data associated with the observation and response.

Dataset generator 1456 can collect and organize collected data 1462 from one or more patients, observations 1464 produced by holistic monitor 1450, and/or responses 1466 as produced by the holistic monitor 1450. In embodiments, dataset generator 1456 can store data, or generate a dataset, with discretized segments corresponding to individual patient profiles. Analysis module 1458 can then analyze the collected data to identify significant trends, characterizations corresponding to specific treatment plans, associated data segments, and insights within the data.

In some embodiments, one or more client devices (e.g., client devices 1430A-B) can be connected to the system 1400. In embodiments, the client device(s) can each include computing devices such as personal computers (PCs), laptops, mobile phones, smart phones, tablet computers, netbook computers, notebook computers, network-connected televisions, etc. In some embodiments, client device(s) can also be referred to as “user devices.”

In embodiments, client devices (e.g., client devices 1430A-B) connected to the system can each include a client application (e.g., client application 1432A-B). In some embodiments, the client application can be an application that provides the user interface (UI) (e.g., client application 1434A-B) and manages transmissions, inputs, and data to and from platform 1420. In some embodiments, the client application that provides the UI can be, or can include, a web browser, a mobile application, a desktop application, etc.

Client devices, under direction by the treatment coordination platform when connected, can present or display a UI (e.g., UI 1434A-B) to a user of the respective client device. In embodiments, the UI can be generated locally at the client device, e.g., through client applications 1432A-B. A UI can include various visual elements and regions, and can be the primary mechanism by which the user interfaces with the client application, the treatment plan coordination platform, and the system at large. In some embodiments, the UI(s) of the client device(s) can include multiple visual elements and regions that enable presentation of information, for decision-making, content delivery, etc. to a user of the device. In some embodiments, the UI can be referred to as a graphical user interface (GUI).

In some embodiments, the system (or any associated platforms), can transmit any data, including audio, video, image, and textual data, to the client device to be interpreted by client application 1432A-B, and displayed via the UI of the respective client device. Such data that can be transmitted to the client device through client applications 1432A-B can include, for example, UI information, textual information, video, or audio streaming data associated with the HCP-patient communications, control, or navigation data, etc. In some embodiments, a client application 1432A-B (e.g., a dedicated application) incorporated within the client devices 1430A-B and can perform function associated with the end-user interface.

In embodiments, connected client devices 1430A-B can also collect input from users through input features. Input features can include UI features, software features, and/or requisite hardware features (e.g., mouse and keyboard, touch screens, etc.) for inputting user requests, and/or data to the treatment plan coordination system. Input features of client devices 1430A-B can include space, regions, or elements of the UI 1434A-B that accept user inputs. For example, input features can be visual elements such as buttons, text-entry spaces, selection lists, drop-down lists, control panels, etc.

In embodiments, connected client devices 1430A-B can also collect input from an associated media system 1436A-B e.g., a camera, microphone, and/or similar elements of a client device, to transmit or intake further user-inputs. In embodiments, the media system of the client device can include at least a display, a microphone, speakers, and a camera, etc., together with other media elements as well. Such elements (e.g., speakers, or a display) can further be used to output data, as well as intake data or inputs.

In embodiments, a client application (e.g., client application 1432A-B) can execute a series of protocols to access and control media system hardware resources, in some cases accessing device-level APIs or drivers that interact with the underlying hardware of a media system. Through such, or similar, protocols, client applications can utilize any of the components of a client device media system for specific functionalities within the context of virtual dental care. For instance, in embodiments, a display of the media system can be employed by the client application (under direction from the treatment coordination platform 1420) to render the UI. In embodiments, graphical elements can be presented or displayed to the user via the display and the UI. The client application of a device can direct rendering commands to the display to update the screen with relevant visual information. Similarly, and/or simultaneously, in embodiments, a camera or imaging sensor of the media system can capture image and/or video input from the user to transmit. In embodiments, the client application can process, encode, and transmit such data from the client device, over the network, to the treatment plan coordination platform 1420.

As will be discussed further below, in embodiments, a client application 1432B associated with a patient client device 1430B can transfer patient data (including captured audio and/or image data), biomarker data, patient observations (e.g., of experienced pain, looseness of aligners, aligner fit, etc.), force or pressure data measured by sensors of palatal expanders, other sensor data measured by sensors of palatal expanders, etc. associated with the treatment plan to treatment plan coordination platform 1420, which can forward, process and/or store such data. In embodiments, such data can be forwarded from a first client device to a second client device.

As will be further discussed below, in embodiments, data collected from a patient client device 1430B can be stored in storage device 1444 as collected data 1462. Such collected data 1462 can include collected data 1462 associated with a single patient, and a single patient dental treatment plan. Alternatively, data associated with multiple patients and/or multiple dental treatment plans and separate procedures can be stored as individual data segments of collected data 1462.

In embodiments, a first client device 1430B can gather data and inputs from a patient, to be transmitted and displayed to an HCP at a second client device 1430A. For instance, in embodiments, client device 1430B can belong to a patient, while client device 1430A can belong to an HCP. In embodiments, such a pairing and configuration can facilitate communication and data transfer between both parties. For example collected patient data from client device 1430B can be transmitted and displayed to an HCP at client device 1430A, which can then transmit instructions, guidance, or any other kinds of data back to the patient client device 1430B. In embodiments, such data can include updates to a treatment plan.

In embodiments, client device 1430B connected to the system 1400 can collect data from more than one sensor, media systems, and/or client device(s). For instance, in embodiments, data can be gathered from an integrated sensors 1436C (e.g., a force sensor) from the palatal expander(s) 1430C. Simultaneously, data can be gathered from the media system 1436B (e.g., from an image sensor) of the client device 1430B. In embodiments, media system 1436B and/or palatal expander 1430C can include one or more sensors external, or wirelessly connected to the devices.

Regardless of the source, sensors and media systems can collected sensor data associated with a single user. For example, in embodiments, both client device 1430B and palatal expander 1430C can be associated with one user. In embodiments, client device 1430B and palatal expander 1430C can be different types of client devices for collecting different types of data. For example, in embodiments, data can be gathered from an integrated camera of the media system 1436B of client device 1430B, while palatal expander 1430C can include one or more dedicated or embedded sensors 1436C (e.g., a MEMS sensor). For example, in some embodiments, client device 1430B can be a personal phone or similar device, while palatal expander 1430C can be a 3D printed palatal expander device placed within a palatal area of a patient. In some embodiments, media system 1436B can access, include, or be a part of an image sensor or scanner for obtaining two-dimensional (2D) data of a dental site in a patient's oral cavity (or another imaging device including a camera) and can be operatively connected to a personal client device (e.g., client device 1430B). In some embodiments, more than two, including any number of, client devices can be used to gather and monitor oral health data from the patient.

Sensors 1436C of palatal expander 1430C can be, for example, sensors of a palatal expander device worn in a mouth of a patient, as described with respect to FIGS. 2A-B. Sensors 1436C can be or include one or more strain gauges or other force sensors for detecting an amount of force exerted on the dental device by one or more teeth of a patient, or conversely, an amount of force exerted on the one or more teeth of the patient by the dental device. In some examples, the palatal expansion device can include a sensor for determining an amount of tooth movement (e.g., optical, or electrical sensor pairs places on opposing sides of an intraoral cavity that can determine distances between them to determine displacement, capacitance sensors that can measure distances). In another example, sensors 1436C can be a sensor that measures properties such as pH, bio indicators or biomarkers, saliva quantity, temperature, and so on. Any and all such sensor data can be collected by sensors 1436C. Such sensor data can be provided to client device 1430B for forwarding on to treatment plan coordination platform 1420 and/or can be provided directly to treatment plan coordination platform 1420 via network 1401. More information about sensors that can be included in dental devices for collecting data is described in U.S. Pat. No. 10,470,847, issued Nov. 12, 2019 (including various types of sensors for compliance detection), U.S. Pat. No. 11,779,243, issued Oct. 10, 2023 (including capacitance sensors in microfluidic arrays to detect stress relaxation), U.S. Pat. No. 11,576,766, issued Feb. 14, 2023 (including biosensors derive tooth movement based on detected biomarkers, e.g., cytokines), U.S. Pat. No. 11,432,908, issued Sep. 6, 2022 (including sensors to detect force/impedance), and U.S. Pat. No. 7,854,609, issued Dec. 21, 2010 (including reservoirs with an agent that is released when aligner is worn, and the agent concentration is measured to determine wear time), each of which is incorporated by reference herein in its entirety.

In embodiments, palatal expander 1430C can gather and transmit patient data without a traditional display or UI. For example, in embodiments, diagnostics monitoring palatal expander 1430C can be placed in a palatal area of the patient. E.g., in embodiments, diagnostics monitoring palatal expander 1430C can be, or be a part of a dental appliance, fixture, or apparatus.

In embodiments, diagnostics monitoring palatal expander 1430C can be equipped with various sensors 1436C, which can be specific types of input features, including any kind of sensors and input mechanisms for gathering patient data. For example, in embodiments, the device can include biological sensors for capturing real-time measurements (e.g., collected data) associated with a patient. In embodiments, such sensors can include pressure sensors (including touch or tactile sensors), motion sensors (e.g., accelerometers, vibration sensors, etc.), audio sensors (e.g., microelectromechanical system (MEMS) microphones, biomarker sensors, chemical sensors (e.g., such as a pH sensor), optical sensors (e.g., such as a color sensors or light sensors), image sensors, temperature sensors, heart-rate sensors, electrical sensors (e.g., capacitive, resistive, conductive, etc.), electrodes, proximity sensors, or any combination of such or similar sensors to gather sensor data associated with tooth position, movement, or any other data relevant to a dental treatment plan.

Examples of proximity sensors suitable for use with the embodiments herein include capacitive sensors, resistive sensors, inductive sensors, eddy-current sensors, magnetic sensors, optical sensors, image sensors, ultrasonic sensors, Hall Effect sensors, infrared touch sensors, or surface acoustic wave (SAW) touch sensors. In embodiments, a proximity sensor can be activated when within a certain distance of the sensing target. The distance can be about less than 1 mm, or within a range from about 1 mm to about 50 mm. In some embodiments, a proximity sensor can be activated without direct contact between the sensor and the sensing target (e.g., the maximum sensing distance is greater than zero).

In some embodiments, a proximity sensor is activated when in direct contact with the sensing target (the sensing distance is zero), also known as a touch or tactile sensor. Examples of touch sensors include capacitive touch sensors, resistive touch sensors, inductive sensors, pressure sensors, and force sensors. In some embodiments, a touch sensor is activated only by direct contact between the sensor and the sensing target (e.g., the maximum sensing distance is zero). Some of the proximity sensor types described herein (e.g., capacitive sensors) can also be touch sensors, such that they are activated both by proximity to the sensing target as well as direct contact with the target.

One or more proximity sensors can be integrated in the palatal expander and used to detect whether the appliance is in proximity to one or more sensing targets. The sensing targets can be an intraoral tissue (e.g., the teeth, gingiva, palate, lips, tongue, cheeks, or a combination thereof). For example, proximity sensors can be positioned on the buccal and/or lingual surfaces of palatal expander 1430C in order to detect features such as tooth position, movement, or dental appliance usage (e.g., compliance) based on proximity to and/or direct contact with the patient's cheeks and/or tongue. As another example, one or more proximity sensors can be positioned in the palatal expander so as to detect features such as tooth position, movement, or dental appliance usage (e.g., compliance) based on proximity to and/or direct contact with the enamel and/or gingiva. In some embodiments, multiple proximity sensors are positioned at different locations appliance so as to detect proximity to and/or direct contact with different portions of the intraoral cavity.

Alternatively or in combination, one or more sensing targets can be coupled to an intraoral tissue (e.g., integrated in an attachment device on a tooth), or can be some other component located in the intraoral cavity (e.g., a metallic filling). Alternatively or in combination, one or more proximity sensors can be located in the intraoral cavity (e.g., integrated in an attachment device on a tooth) and the corresponding sensing target(s) can be integrated in the intraoral appliance. Optionally, a proximity sensor integrated in a first appliance on a patient's upper or lower jaw can be used to detect a sensing target integrated in a second appliance on the opposing jaw or coupled to a portion of the opposing jaw (e.g., attached to a tooth), and thus detect proximity and/or direct contact between the patient's jaws.

The proximity sensor can be a capacitive sensor activated by charges on the sensing target. The capacitive sensor can be activated by charges associated with intraoral tissues or components such as the enamel, gingiva, oral mucosa, saliva, cheeks, lips, and/or tongue. For example, the capacitive sensor can be activated by charges (e.g., positive charges) associated with plaque and/or bacteria on the patient's teeth or other intraoral tissues. In such embodiments, the capacitive sensing data can be used to determine whether the appliance is being worn, and optionally the amount of plaque and/or bacteria on the teeth. As another example, the capacitive sensor can be activated by charges associated with the crowns of teeth, e.g., negative charges due to the presence of ionized carboxyl groups covalently bonded to sialic acid.

Alternatively or in combination, the intraoral tissue can serve as the ground electrode of the capacitive sensor. Optionally, a shielding mechanism can be used to guide the electric field of the capacitive sensor in a certain location and/or direction for detecting contact with a particular tissue.

Alternatively or in combination, a monitoring palatal expander can include one or more vibration sensors configured to generate sensor data indicative of intraoral vibration patterns. Examples of vibration sensors include audio sensors (e.g., MEMS microphones), accelerometers, and piezoelectric sensors. The intraoral vibration patterns can be associated with one or more of: vibrations transferred to the patient's teeth via the patient's jawbone, teeth grinding, speech, mastication, breathing, or snoring. In some embodiments, the intraoral vibration patterns originate from sounds received by the patient's ear drums. The intraoral vibration patterns can also originate from intraoral activities, such as teeth grinding, speech, mastication, breathing, snoring, etc. The sensor data generated by the vibration sensors can be processed to detect features such as tooth position, movement, or dental appliance usage (e.g., compliance). For instance, the palatal expander can include a processor that compares the detected intraoral vibration patterns to patient-specific intraoral vibration patterns to determine whether the appliance is being worn on a patient's teeth. In some embodiments, the processor is trained using previous data of patient-specific intraoral vibration patterns, and then determines whether the appliance is being worn by matching the measured patterns to the previous patterns. Alternatively or in combination, appliance usage can be determined by comparing the measured vibration patterns to vibration patterns obtained when the appliance is not being worn.

Alternatively or in combination, a palatal expander can include one or more optical sensors or image sensors configured to detect features such as tooth position, movement, or dental appliance usage (e.g., compliance) based on optical signals. For example, the optical sensors can be color sensors (e.g., mono-channel color sensors, multi-channel color sensors such as RGB sensors) configured to detect the colors of intraoral tissues. In some embodiments, one or more color sensors can be integrated into the intraoral appliance so as to be positioned adjacent to certain intraoral tissue (e.g., enamel, gingiva, cheeks, tongue, etc.) when the appliance is worn in the mouth. The device can capture data associated with dentition as well as whether the appliance is currently being worn based on the types and visibility of colors detected by the sensors. In such embodiments, the monitoring device can include one or more light sources (e.g., LEDs) providing illumination for the color sensors.

As another example, the palatal expander can include one or more emitters (e.g., a LED) configured to generate optical signals and one or more optical sensors (e.g., a photodetector) configured to measure the optical signals. For example, an emitter can be positioned such that when the appliance is worn, the optical signal is reflected off of a surface (e.g., an intraoral tissue, a portion of an intraoral appliance) in order to reach the corresponding optical sensor. In some embodiments, when the appliance is not being worn, the optical signal is not reflected and does not reach the optical sensor. Accordingly, data indicative of a patient's detention, as well as compliance data, can be determined via aspects of data captured via the optical sensor.

Alternatively or in combination, the palatal expander of the present disclosure can include one or more magnetic sensors configured to detect appliance usage based on changes to a magnetic field. Examples of magnetic sensors suitable for use with the embodiments herein include magnetometers, Hall Effect sensors, magnetic reed switches, and magneto-resistive sensors. In some embodiments, the characteristics of the magnetic field (e.g., magnitude, direction) vary based on whether the appliance is currently being worn, proximity of teeth, etc., e.g., due to interference from intraoral tissues such as the teeth. Accordingly, the device can determine appliance usage and dentition data by processing and analyzing the magnetic field detected by the magnetic sensors.

Alternatively or in combination, a palatal expander can utilize two or more magnets that interact with each other (e.g., by exerting magnetic forces on each other), and a sensor that detects the interaction between the magnets. For example, the sensor can be a mechanical switch coupled to a magnet and actuated by magnetic forces exerted on the magnet. As another example, the sensor can be configured to detect the characteristics (e.g., magnitude, direction) of the magnetic force exerted on a magnet by the other magnets. The magnets and sensor can each be independently integrated within a dental appliance or coupled to a tooth or other intraoral tissue to gather data associated with dentition and compliance.

In embodiments, a palatal expander can include sensors for capture of data associated with bioagents in the intraoral cavity while an intra-oral appliance (e.g., aligner, palatal expander, etc.) is in use. Such apparatuses and methods can collect information (data), including data about tooth movement stages, via analysis of biomarkers in saliva or gingival crevicular fluid (GCF). For example, the data can be indicative of patient wearing compliance, the amount of tooth movement achieved, the amount of force and/or pressure actually applied to the teeth by the appliance, bone remodeling processes and stages, tissue health, bacterial activity in the oral cavity, or any combination thereof.

In embodiments, one or more sensors of sensors 136C can be positioned to collect data from a specific portion of a patient's mouth. For instance, in embodiments, a sensor can be located at any portion of palatal expander 1430C, such as at or near a distal portion, a mesial portion, a buccal portion, a lingual portion, a gingival portion, an occlusal portion, or a combination thereof of the palatal expander. In embodiments, a sensor can be positioned near a tissue of interest when the appliance is worn in the patient's mouth, such as near or adjacent the teeth, gingiva, palate, lips, tongue, cheeks, airway, or a combination thereof. For example, when the appliance is worn, the sensor can cover a single tooth, or a portion of a single tooth. Alternatively, the sensor can cover multiple teeth or portions thereof. In embodiments where multiple sensors are used, some or all of the monitoring devices can be located at different portions of the appliance and/or intraoral cavity. Alternatively, some or all of the sensors can be located at the same portion of the appliance and/or intraoral cavity.

In embodiments, sensors 1436C can be of any kind to collect any kind of data associated with a patient's oral health, and/or data that is relevant for a dental treatment plan. Further specific data types that can be collected from such sensors will be described in further detail below, with respect to the description of collected data 1462.

Once the palatal expander 1430C has gathered the patient data, such data can be transmitted to the treatment plan coordination platform 1420. In embodiments, palatal expander 1430C can include an onboard data processing module for standardizing, encrypting, and transmitting such data to platform 1420. In embodiments, palatal expander 1430C can transmit such data first to a separate client device of the user (e.g., client device 1430B), which can then transmit the data to treatment plan coordination platform 1420. For example, in any number of connected client devices (e.g., sensor device 1430C) collected data can be stored in physical memory on the device and can be retrieved by another device (e.g., client device 1430A) in communication with the monitoring apparatus. Retrieval can be done wirelessly, e.g., using near-field communication (NFC) and/or Bluetooth (BLE) technologies to use a smartphone or other hand-held device to retrieve the data.

Thus, any client device used for diagnostics monitoring, including a palatal expander 1430C, can include the requisite hardware for enabling such transmissions of collected data. Such hardware can include requisite sensors as have been described, a CPU, an NFC communication module, an NFC antenna, a PCB, a battery, etc. In embodiments, client devices can further be or include cases or holders that can boost and/or relay the signals from the sensing portion of a device to a handheld device such as a smartphone; such cases or holders can be referred to as NFC-BLE enabled cases.

Thus, in some embodiments, such data be gathered remotely e.g., via a patient at their home (alternatively, such data can be collected at a clinic e.g., at an HCP's office). As previously mentioned, through such multiple, connected, client devices of the treatment plan coordination system, real-time or near-real-time collection and transmission of patient data can be accomplished, thus enhancing data collection while minimizing intrusiveness into a patient's lifestyle.

In embodiments herein, such patient data collected by a client device of a user, as described with respect to palatal expander 1430C and/or devices 1430A-B, can be holistically referenced as collected data 1462, which can be ultimately stored within storage device 1444.

In some embodiments, the system can include storage platform 1440, which can host and manage storage device 1444. In some embodiments, platform 1440 can be a dedicated server for supporting storage device 1444 accessible via network 1401.

In embodiments, collected data 1462 can include any data that has been collected from client devices associated with the system. In embodiments, the collected data 1462 can be data collected from one or more patient's before, after, or during a dental treatment plan. In embodiments, such data can be accessible and displayable via any of the connected client devices.

In embodiments, collected data 1462 can include the oral health data acquired through multiple sources, such as from sensors of a palatal expander and/or a connected device (as described above). In embodiments, these can be at least client device 1430A and palatal expander 1430C. In embodiments, such collected data 1462 can include oral temperature data, oral pH data, pressure data (e.g., bite force or pressure otherwise sensed by a specific tooth, etc.), image data (including x-ray, or ultrasound, etc.), biomarker data, heart rate data, respiratory data, body temperature, image data, video data, textual data, and or raw data indicative of electrical parameters associated with health data (e.g., capacitance, resistance, conductance values, etc.). In embodiments, collected data 1462 can be any kind of data associated with a patient's oral health, and/or data that is relevant for a treatment plan.

In embodiments, such collected data can include spatial positioning data, including 2D or 3D patient data. In embodiments, collected data can include image data which can be used to generate a virtual model (e.g., a virtual 2D model or virtual 3D model) of the real-time conditions of the patient's oral features and/or dentition (e.g., conditions of a tooth, or a dental arch, etc., can be modeled).

In embodiments, storage device 1444 can further include an initial treatment plan 1460, and observations 1464 and responses 1466, as produced by holistic monitor 1450 (and/or analysis module 1458).

In embodiments, the initial treatment plan 1460 can function as, or be an initial, pre-defined treatment plan that consists of scheduled stages designed to sequentially correct and improve aspects of a patient's health. In some embodiments, the initial treatment plan can be a plan for improving aspects of a patient's oral health. In some cases, the plan can be an initial plan determined by an HCP, and based on portions of collected data 1462, such as tests, documentation, medical history, etc. For example, in embodiments the initial treatment plan can be a multi-stage palatal expansion treatment plan initially been generated by an HCP (e.g., an orthodontist) after performing a scan of an initial pre-treatment condition of the patient's dental arch. In some embodiments, the initial treatment plan can begin at home (e.g., be based on a patient scan of his- or her-self) or at a scanning center. In embodiments, the initial treatment plan might be created automatically and/or by a professional (including an orthodontist) in a remote service center.

In embodiments, the initial dental treatment plan can be a palatal expansion treatment plan based on intraoral scan data providing surface topography data for the patient's intraoral cavity (including teeth, gingival tissues, etc.). Such surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.), as was previously described. One of ordinary skill in the art, having the benefit of this disclosure, will appreciate that numerous methods, mechanisms, and strategies for generating an initial dental treatment plan exist, and that the discussed methods represent exemplary methods, mechanisms, and strategies for generating an initial dental treatment plan associated with the system.

In embodiments, a palatal expansion treatment plan can be associated with any orthodontic procedure. Such a procedure can refer to, inter alia, any procedure involving the oral cavity and directed to the design, manufacture, or installation of orthodontic elements at a dental site within the oral cavity, or a real or virtual model thereof, or directed to the design and preparation of the dental site to receive such orthodontic elements. Such elements can be appliances including but not limited to brackets and wires, retainers, aligners, or functional appliances. In embodiments, various palatal expander devices can be formed, or one device can be modified, for each treatment stage to provide forces to move the patient's teeth or jaw. The shape of such palatal expander device(s) can unique and customized for a particular patient and a particular treatment stage.

In embodiments, one or more stages of the dental and/or orthodontic treatment plan can correspond to a specific palatal expander that the patient must wear for a predetermined period. In some embodiments, such a period, or time interval, can range from one day to three weeks. For example, in some cases, the treatment can begin with the first palatal expander, tailored to fit a patient's current dental configuration. Such an initial palatal expander can apply targeted pressure on regions of the patient's palate, initiating the process of gradual expansion. Once the patient has worn this initial palatal expander for the duration specified in the first stage of the initial treatment plan 1460, the patient can transition to subsequent stage (e.g., the subsequent stage in a sequence of stages). This can involve replacing the initial palatal expander with a new one, designed to continue the process of expansion. This can involve the modification or adjustment of the initial, or some, of the provided palatal expanders. Subsequent stages can introduce a new palatal expander, manufactured to incrementally move teeth, and expand the palate closer to the desired final position.

Such an initial treatment plan 1460 can include checkpoints or assessment periods, where HCPs and/or dental professionals assess the progress of the treatment. During such checkpoints, digital scans, images, molds, etc., can be taken to ensure that the palate is expanding according to the planned trajectory. In embodiments, such checkpoints, or assessments, can occur during or in between stages of a given dental treatment plan. In embodiments, the dental treatment plan can prescribe, or outline specific time intervals between checkpoints. In some embodiments, any of the previously discussed collected data types can be collected during such checkpoints.

In some embodiments, any, or all of data within storage device 1444 can be accessed and modified by treatment coordination platform 1420 (or other modules and platforms of the system), for further processing.

As was discussed, in some embodiments, analysis module 1458 can include an AI model to analyze collected data 1462, and produce treatment plans or updates.

In embodiments, such an AI model can be one or more of decision trees (e.g., random forests), support vector machines, logistic regression, K-nearest neighbor (KNN), or other types of machine learning models, for example. In one embodiment, such an AI model can be one or more artificial neural networks (also referred to simply as a neural network). The artificial neural network can be, for example, a convolutional neural network (CNN) or a deep neural network.

In one embodiment, processing logic performs supervised machine learning to train the neural network.

In some embodiments, the artificial neural network(s) can generally include a feature representation component with a classifier or regression layers that map features to a target output space. A convolutional neural network (CNN), for example, can host multiple layers of convolutional filters. Pooling can be performed, and non-linearities can be addressed, at lower layers, on top of which a multi-layer perceptron is commonly appended, mapping top layer features extracted by the convolutional layers to decisions (e.g., classification outputs). The neural network can be a deep network with multiple hidden layers or a shallow network with zero or a few (e.g., 1-2) hidden layers. Deep learning is a class of machine learning algorithms that use a cascade of multiple layers of nonlinear processing units for feature extraction and transformation. Each successive layer uses the output from the previous layer as input. Neural networks can learn in a supervised (e.g., classification) and/or unsupervised (e.g., pattern analysis) manner. Some neural networks (e.g., such as certain deep neural networks) can include a hierarchy of layers, where the different layers learn different levels of representations that correspond to different levels of abstraction. In deep learning using such networks, each level can learn to transform its input data into a slightly more abstract and composite representation. In embodiments of such neural networks, such layers may not be hierarchically arranged (e.g., such neural networks can include structures that differ from a traditional layer-by-layer approach).

In some embodiments, such an AI model can be one or more recurrent neural networks (RNNs). An RNN is a type of neural network that includes a memory to enable the neural network to capture temporal dependencies. An RNN is able to learn input-output mappings that depend on both a current input and past inputs. The RNN will address past and future measurements and make predictions based on this continuous measurement information. One type of RNN that can be used is a long short-term memory (LSTM) neural network.

As indicated above, such an AI model can include one or more generative AI models, allowing for the generation of new and original content, such a generative AI model can include aspects of a transformer architecture, or a generative adversarial network (GAN) architecture. Such a generative AI model can use other machine learning models including an encoder-decoder architecture including one or more self-attention mechanisms, and one or more feed-forward mechanisms. In some embodiments, the generative AI model can include an encoder that can encode input textual data into a vector space representation; and a decoder that can reconstruct the data from the vector space, generating outputs with increased novelty and uniqueness. The self-attention mechanism can compute the importance of phrases or words within a text data with respect to all of the text data. A generative AI model can also utilize the previously discussed deep learning techniques, including recurrent neural networks (RNNs), convolutional neural networks (CNNs), or transformer networks. Further details regarding generative AI models are provided herein.

In some embodiments, storage device 1444 can be hosted by one or more storage devices, such as main memory, magnetic or optical storage-based disks, tapes or hard drives, network-attached storage (NAS), storage area network (SAN), and so forth. In some embodiments, storage device 1444 can be a network-attached file server, while in other embodiments, storage device 1444 can be or can host some other type of persistent storage such as an object-oriented database, a relational database, and so forth.

In some embodiments, storage device(s) 1444 can be hosted by any of the platforms or device associated with system 1400 (e.g. treatment plan coordination platform 1420). In other embodiments, storage device 1444 can be on or hosted by one or more different machines coupled to the treatment coordination platform via network 1401. In some cases, the storage device 1444 can store portions of audio, video, image, or text data received from the client devices (e.g., client device 1430A-B) and/or any platform and any of its associated modules.

In some embodiments, any one of the associated platforms (e.g., treatment plan coordination platform 1420) can temporarily accumulate and store data until it is transferred to storage devices 1444 for permanent storage.

It is appreciated that in some implementations, the functions of platforms 1420 and/or 1440 can be provided by a fewer number of machines. For example, in some implementations, functionalities of platforms 1420 and/or 1440 can be integrated into a single machine, while in other implementations, functionalities of platforms 1420 and/or 1440 can be integrated into multiple, or more, machines. In addition, in some implementations, only some platforms of the system can be integrated into a combined platform.

While the modules of each platform are described separately, it should be understood that the functionalities can be divided differently or integrated in various ways within the platform while still applying similar functionality for the system. Furthermore, each platform and associated modules can be implemented in various forms, such as standalone applications, web-based platforms, integrated systems within larger software suites, or dedicated hardware devices, just to name a few possible forms.

In general, functions described in embodiments as being performed by platforms 1420, 1440, and/or holistic monitor 1450 can also be performed by client devices (e.g., client device 1430A, client device 1430B). In addition, the functionality attributed to a particular component can be performed by different or multiple components operating together. Platforms 1420, 1440, and/or holistic monitor 1450 can also be accessed as a service provided to other systems or devices through appropriate application programming interfaces, and thus is not limited to use in websites.

It is appreciated that in some implementations, platforms 1420, 1440, and/or holistic monitor 1450 or client devices of the system (e.g., client device 1430A, client device 1430B) and/or storage device 1444, can each include an associated API, or mechanism for communicating with APIs. In such a way, any of the components of system 1400 can support instructions and/or communication mechanisms that can be used to communicate data requests and formats of data to and from any other component of system 1400, in addition to communicating with APIs external to the system (e.g., not shown in FIG. 14).

In some embodiments of the disclosure, a “user” can be represented as a single individual. However, other implementations of the disclosure encompass a “user” being an entity controlled by a set of users and/or an automated source. For example, a set of individual users federated as a community in a social network can be considered a “user.” In another example, an automated consumer can be an automated ingestion pipeline, such as a topic channel.

In situations in which the systems, or components therein, discussed here collect personal information about users, or can make use of personal information, the users can be provided with an opportunity to control whether the system or components collect user information (e.g., information about a user's social network, social actions or activities, profession, a user's preferences, or a user's current location), or to control whether and/or how to receive content from the system or components that can be more relevant to the user. In addition, certain data can be treated in one or more ways before it is stored or used, so that personally identifiable information is removed. For example, a user's identity can be treated so that no personally identifiable information can be determined for the user, or a user's geographic location can be generalized where location information is obtained (such as to a city, ZIP code, or state level), so that a particular location of a user cannot be determined. Thus, the user can have control over how information is collected about the user and used by the system and components.

FIG. 15 shows a block diagram of an example system for virtual dental care for palatal expansion, in accordance with some embodiments. As shown in FIG. 15, system 1500 can include a dental consumer/patient system 1502, a dental professional system 1550, a virtual dental care system 1506, and a network 1504. The dental consumer/patient system 1502, dental professional system 1550, and virtual dental care system 1506 can communicate to one another over the network 1504, which can include one or more local area networks (LANs), public wide area networks (e.g., such as the Internet) and/or private wide area networks (e.g., Intranets), one or more personal Area Networks (PAN), one or more cellular networks (e.g., a Global System for Mobile Communications (GSM) network), and/or any other suitable network.

Dental consumer/patient system 1502 generally represents any type or form of computing device capable of reading computer-executable instructions. Dental consumer/patient system 1502 can be, for example, a desktop computer, a tablet computing device, a laptop, a smartphone, an augmented reality device, or other consumer device. Additional examples of dental consumer/patient system 1502 include, without limitation, laptops, tablets, desktops, servers, cellular phones, Personal Digital Assistants (PDAs), multimedia players, embedded systems, wearable devices (e.g., smart watches, smart glasses, etc.), smart vehicles, smart packaging (e.g., active or intelligent packaging), gaming consoles, Internet-of-Things devices (e.g., smart appliances, etc.), variations or combinations of one or more of the same, and/or any other suitable computing device. The dental consumer/patient system 1502 need not be or include a clinical scanner (e.g., an intraoral scanner), though it is contemplated that in some implementations the functionalities described herein in relation to the dental consumer/patient system 1502 can be incorporated into a clinical scanner. As an example of various implementations, a camera 1532 of the dental consumer/patient system 1502 can comprise an ordinary camera that captures 2D images of the patient's dentition and does not capture height-map and/or other data (e.g., three-dimensional (3D) data) that is used to stitch a mesh of a 3D surface. In some examples, the dental consumer/patient system 1502 can include an at-home intraoral scanner.

In some implementations, the dental consumer/patient system 1502 is configured to interface with a dental consumer and/or dental patient. A “dental consumer,” as used herein, can include a person seeking assessment, diagnosis, and/or treatment for a dental condition (general dental condition, orthodontic condition, endodontic condition, condition requiring restorative dentistry, etc.). A dental consumer can, but need not, have agreed to and/or started treatment for a dental condition. A “dental patient,” as used herein, can include a person who has agreed to diagnosis and/or treatment for a dental condition. A dental consumer and/or a dental patient, can, for instance, be interested in and/or have started orthodontic treatment, such as treatment using one or more (e.g., a sequence of) aligners (e.g., polymeric appliances having a plurality of tooth-receiving cavities shaped to successively reposition a person's teeth from an initial arrangement toward a target arrangement). In various implementations, the dental consumer/patient system 1502 provides a dental consumer/dental patient with software (e.g., one or more webpages, standalone applications, mobile applications, etc.) that allows the dental consumer/patient to capture images of their dentition, interact with dental professionals (e.g., users of the dental professional system 1550), manage treatment plans (e.g., those from the virtual dental care system 1506 and/or the dental professional system 1550), and/or communicate with dental professionals (e.g., users of the dental professional system 1480).

Dental professional system 1550 generally represents any type or form of computing device capable of reading computer-executable instructions. Dental professional system 1550 can be, for example, a desktop computer, a tablet computing device, a laptop, a smartphone, an augmented reality device, or other consumer device. Additional examples of dental professional system 1550 include, without limitation, laptops, tablets, desktops, servers, cellular phones, Personal Digital Assistants (PDAs), multimedia players, embedded systems, wearable devices (e.g., smart watches, smart glasses, etc.), smart vehicles, smart packaging (e.g., active or intelligent packaging), gaming consoles, Internet-of-Things devices (e.g., smart appliances, etc.), variations or combinations of one or more of the same, and/or any other suitable computing device.

In various implementations, the dental professional system 1550 is configured to interface with a dental professional. A “dental professional” (used interchangeably with dentist, orthodontist, and doctor herein) as used herein, can include any person with specialized training in the field of dentistry, and can include, without limitation, general practice dentists, orthodontists, dental technicians, dental hygienists, etc. A dental professional can include a person who can assess, diagnose, and/or treat a dental condition. “Assessment” of a dental condition, as used herein, can include an estimation of the existence of a dental condition. An assessment of a dental condition need not be a clinical diagnosis of the dental condition. In some embodiments, an “assessment” of a dental condition can include an “image-based assessment,” that is an assessment of a dental condition based in part or on whole on photos and/or images (e.g., images that are not used to stitch a mesh or form the basis of a clinical scan) taken of the dental condition. A “diagnosis” of a dental condition, as used herein, can include a clinical identification of the nature of an illness or other problem by examination of the symptoms. “Treatment” of a dental condition, as used herein, can include prescription and/or administration of care to address the dental conditions. In particular, embodiments are directed to prescription and/or administration of treatment with respect to palatal expansion. The dental professional system 1550 can provide to a user software (e.g., one or more webpages, standalone applications (e.g., dedicated treatment planning and/or treatment visualization applications), mobile applications, etc.) that allows the user to interact with users (e.g., users of the dental consumer/patient system 1502, other dental professionals, etc.), create/modify/manage treatment plans (e.g., those from the virtual dental care system 1506 and/or those generated at the dental professional system 1550), etc.

Virtual dental care system 1506 generally represents any type or form of computing device that is capable of storing and analyzing data. Virtual dental care system 1506 can include a backend database server for storing patient data and treatment data. Additional examples of virtual dental care system 1506 include, without limitation, security servers, application servers, web servers, storage servers, and/or database servers configured to run certain software applications and/or provide various security, web, storage, and/or database services. Although illustrated as a single entity in FIG. 15, virtual dental care system 1506 can include and/or represent a plurality of servers that work and/or operate in conjunction with one another.

As illustrated in FIG. 15, dental consumer/patient system 1502, virtual dental care system, 1506, and/or dental professional system 1550 can include one or more memory devices, such as memory 1540. Memory 1540 generally represents any type or form of volatile or non-volatile storage device or medium capable of storing data and/or computer-readable instructions. In one example, memory 1540 can store, load, execute in conjunction with physical processor(s) 1530, and/or maintain one or more of virtual dental care modules 1508. Examples of memory 1540 include, without limitation, Random Access Memory (RAM), Read Only Memory (ROM), flash memory, Hard Disk Drives (HDDs), Solid-State Drives (SSDs), optical disk drives, caches, variations, or combinations of one or more of the same, and/or any other suitable storage memory.

As illustrated in FIG. 15, dental consumer/patient system 1502, dental professional system 1550, and/or virtual dental care system 1506 can also include one or more physical processors, such as physical processor(s) 1530. Physical processor(s) 1530 generally represents any type or form of hardware-implemented processing unit capable of interpreting and/or executing computer-readable instructions. In one example, physical processor(s) 1530 can access and/or modify one or more of virtual dental care modules 1508 stored in memory 1540. Additionally or alternatively, physical processor 1530 can execute one or more of virtual dental care modules 1508 to facilitate preamble phrase. Examples of physical processor(s) 1530 include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, and/or any other suitable physical processor.

In some embodiments, dental consumer/patient system 1502 can include a camera 1532. Camera 1532 can comprise a camera, scanner, or other optical sensor. Camera 1532 can include one or more lenses or can, one or more camera devices, and/or one or more other optical sensors. In some examples, camera 1532 can include other sensors and/or devices which can aid in capturing optical data, such as one or more lights, depth sensors, etc. In various implementations, the camera 1532 is not a clinical scanner.

Virtual dental care datastore(s) 1520 include one or more datastore configured to store any type or form of data that can be used for virtual dental care. In some embodiments, the virtual dental care datastore(s) 1520 include, without limitation, patient data 1536 and treatment data 1538. Patient data 1536 can include data collected from patients, such as patient dentition information, patient historical data, patient scans, patient information, etc. Treatment data 1538 can include data used for treating patients, such as treatment plans, state of treatment, success of treatment, changes to treatment, notes regarding treatment, etc.

As will be described in greater detail below, one or more of virtual dental care modules 1508 and/or the virtual dental care datastore(s) 1520 in FIG. 15 can, (when executed by at least one processor of dental consumer/patient system 1502, virtual dental care system 1506, and/or dental professional system 1550) enable dental consumer/patient system 1502, virtual dental care system 1506, and/or dental professional system 1550 to optimize palatal expansion treatment of an existing palatal expansion treatment plan and/or generate a new palatal expansion treatment plan.

Some embodiments provide patients with “Virtual dental care.” “Virtual dental care,” as used herein, can include computer-program instructions and/or software operative to provide remote dental services by a health professional (dentist, orthodontist, dental technician, etc.) to a patient, a potential consumer of dental services, and/or other individual. Virtual dental care can comprise computer-program instructions and/or software operative to provide dental services without a physical meeting and/or with only a limited physical meeting. As an example, virtual dental care can include software operative to providing dental care from the dental professional system 1550 and/or the virtual dental care system 1506 to the computing device 1502 over the network 1504 through e.g., written instructions, interactive applications that allow the health professional and patient/consumer to interact with one another, telephone, chat etc. Some embodiments provide patients with “Remote dental care.” “Remote dental care,” as used herein, can comprise computer-program instructions and/or software operative to provide a remote service in which a health professional provides a patient with dental health care solutions and/or services. In some embodiments, the virtual dental care facilitated by the elements of the system 1500 can include non-clinical dental services, such as dental administration services, dental training services, dental education services, etc.

In some embodiments, the elements of the system 1500 (e.g., the virtual dental care modules 1508 and/or the virtual dental care datastore(s) 1520) can be operative to provide intelligent photo guidance to a patient to take images relevant to virtual dental care using the camera 1532 on the computing device 1502.

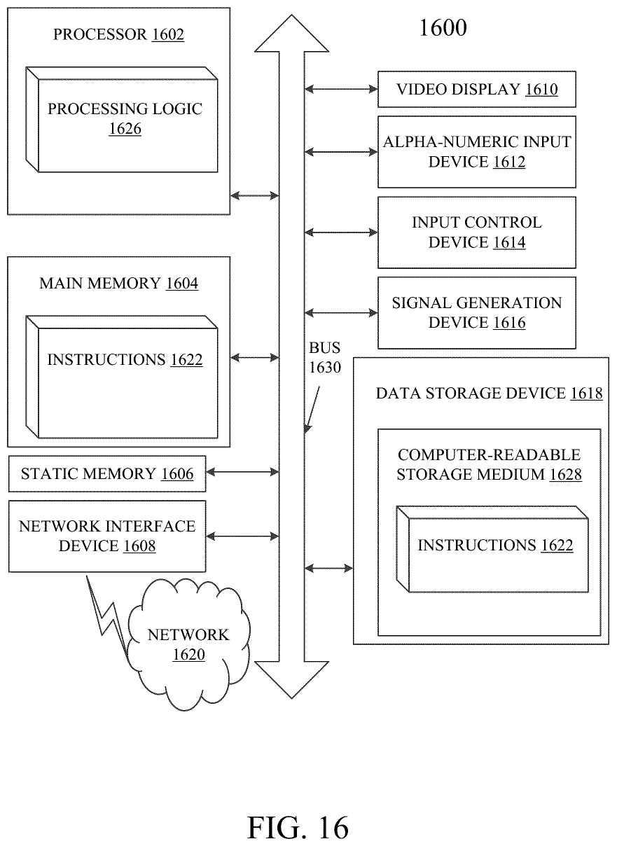

FIG. 16 illustrates a block diagram of an example processing device 1600 operating in accordance with one or more aspects of the present disclosure. In one implementation, the processing device 1600 can be a part of any computing device of FIG. 14, or any combination thereof. Example processing device 1600 can be connected to other processing devices in a LAN, an intranet, an extranet, and/or the Internet. The processing device 1600 can be a personal computer (PC), a set-top box (STB), a server, a network router, switch or bridge, or any device capable of executing a set of instructions (sequential or otherwise) that specify actions to be taken by that device. Further, while only a single example processing device is illustrated, the term “processing device” shall also be taken to include any collection of processing devices (e.g., computers) that individually or jointly execute a set (or multiple sets) of instructions to perform any one or more of the methods discussed herein.

Example processing device 1600 can include a processor 1602 (e.g., a CPU), a main memory 1604 (e.g., read-only memory (ROM), flash memory, dynamic random access memory (DRAM) such as synchronous DRAM (SDRAM), etc.), a static memory 1606 (e.g., flash memory, static random access memory (SRAM), etc.), and a secondary memory (e.g., a data storage device 1618), which can communicate with each other via a bus 1630.

Processor 1602 represents one or more general-purpose processing devices such as a microprocessor, central processing unit, or the like. More particularly, processor 1602 can be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 1602 can also be one or more special-purpose processing devices such as an application specific integrated circuit (ASIC), a field programmable gate array (FPGA), a digital signal processor (DSP), network processor, or the like. In accordance with one or more aspects of the present disclosure, processor 1602 can be configured to execute instructions (e.g. processing logic 1626 can implement the holistic monitor of FIG. 14).

Example processing device 1600 can further include a network interface device 1608, which can be communicatively coupled to a network 1620. Example processing device 1600 can further comprise a video display 1610 (e.g., a liquid crystal display (LCD), a touch screen, or a cathode ray tube (CRT), an alphanumeric input device 1612 (e.g., a keyboard), an input control device 1614 (e.g., a cursor control device, a touch-screen control device, a mouse), and a signal generation device 1616 (e.g., an acoustic speaker).

Data storage device 1618 can include a computer-readable storage medium (or, more specifically, a non-transitory computer-readable storage medium) 1628 on which is stored one or more sets of executable instructions 1622. In accordance with one or more aspects of the present disclosure, executable instructions 1622 can comprise executable instructions (e.g. instructions for implementing the holistic monitor of FIG. 14).

Executable instructions 1622 can also reside, completely or at least partially, within main memory 1604 and/or within processor 1602 during execution thereof by example processing device 1600, main memory 1604 and processor 1602 also constituting computer-readable storage media. Executable instructions 1622 can further be transmitted or received over a network via network interface device 1608.

While the computer-readable storage medium 1628 is shown in FIG. 16 as a single medium, the term “computer-readable storage medium” should be taken to include a single medium or multiple media (e.g., a centralized or distributed database, and/or associated caches and servers) that store the one or more sets of operating instructions. The term “computer-readable storage medium” shall also be taken to include any medium that is capable of storing or encoding a set of instructions for execution by the machine that cause the machine to perform any one or more of the methods described herein. The term “computer-readable storage medium” shall accordingly be taken to include, but not be limited to, solid-state memories, and optical and magnetic media.

The following exemplary embodiments are now described.

Embodiment 1: A method for monitoring palatal expansion, the method comprising: accessing a treatment plan for a patient comprising a series of sequential treatment stages, each of a plurality of the series of sequential treatment stages associated with a particular palatal expander in a series of palatal expanders; receiving one or more two-dimensional (2D) images of an oral cavity of the patient during a treatment stage of the treatment plan; determining, via processing of the one or more 2D images, one or more observations of the oral cavity; determining, based on the one or more observations, a level of progress associated with the treatment plan; and providing a representation of progress corresponding to the determined level of progress.

Embodiment 2: The method of embodiment 1, wherein providing the representation of the progress comprises sending, to a client device, information configured to display the representation on a display of the client device.

Embodiment 3: The method of embodiment 1 or 2, wherein providing the representation of the progress comprises displaying the representation on a display device.

Embodiment 4: The method of embodiments 1-3, wherein the representation of progress comprises at least one of a visual representation that graphically displays the progress, a numerical representation comprising one or more values that indicate the progress, or a textual representation of the progress.

Embodiment 5: The method of embodiments 1-4, further comprising: performing one or more actions based on the determined level of progress.

Embodiment 6: The method of embodiment 5, wherein performing the one or more actions comprises: determining a recommendation based on the determined level of progress; and sending the recommendation to a client device.

Embodiment 7: The method of embodiment 6, wherein the recommendation is a recommendation for an update to the treatment plan, the update determined based on the determined level of progress.

Embodiment 8: The method of embodiment 6 or 7, wherein the recommendation comprises a recommendation to change a schedule for image-based monitoring of the oral cavity of the patient.

Embodiment 9: The method of embodiments 6-8, wherein the recommendation comprises a recommendation to change a schedule of when to proceed from a current stage of the treatment plan to a next stage of the treatment plan.

Embodiment 10: The method of embodiments 1-9, wherein the one or more observations comprise a metric characterizing a level of expansion achieved by the series of palatal expanders.

Embodiment 11: The method of embodiment 10, wherein determining the metric characterizing the level of expansion achieved by the palatal expander comprises: determining a measurement of a feature in the one or more 2D images in units for digital image measurement; and converting the measurement from the units for digital image measurement into units for physical measurement.

Embodiment 12: The method of embodiment 11, further comprising: registering the one or more 2D images to at least one of an image or a three-dimensional (3D) model comprising one or more teeth of the patient that have known physical measurements; and determining a conversion factor for converting between the units for digital image measurement and the units for physical measurement based on a result of the registering.

Embodiment 13: The method of embodiments 1-12, further comprising: determining, based on the one or more observations, an indication of an occurrence of an adverse event associated with the oral cavity.

Embodiment 14: The method of embodiment 13, wherein determining an indication of an occurrence of an adverse event associated with the oral cavity comprises: segmenting the one or more 2D images into a plurality of segments, wherein each segment of the plurality of segments corresponds to a tooth of the patient; and determining a change in a spatial arrangement of the plurality of segments based on a comparison of the spatial arrangement of the plurality of segments to a spatial arrangement of a previously segmented plurality of segments, wherein the previously segmented plurality of segments correspond to one or more previously captured images.

Embodiment 15: The method of embodiment 14, wherein the adverse event comprises increased buccal tipping of one or more teeth of the patient.

Embodiment 16: The method of embodiments 14-15, wherein the adverse event comprises unplanned intrusion, eruption, or exfoliation of one or more teeth of the patient.

Embodiment 17: The method of embodiments 13-16, wherein determining an indication of an occurrence of an adverse event associated with the oral cavity comprises: segmenting the one or more 2D images into a plurality of segments, wherein each segment of the plurality of segments corresponds to a tooth of the patient; and determining a change in a spatial arrangement of the plurality of segments based on a comparison of the spatial arrangement of the plurality of segments to an expected spatial arrangement of the plurality of segments for a current stage of treatment, wherein the expected spatial arrangement is based on the treatment plan.

Embodiment 18: The method of embodiments 13-17, wherein determining an indication of an occurrence of an adverse event associated with the oral cavity comprises identifying a presence of soft tissue damage.

Embodiment 19: The method of embodiments 13-18, wherein determining an indication of an occurrence of an adverse event associated with the oral cavity comprises identifying a presence of anterior or posterior impingement of soft tissue by the particular palatal expander.

Embodiment 20: The method of embodiments 1-19, wherein the one or more 2D images comprise a 2D image of a palatal expander in an upper palate of the patient, the method further comprising: determining, based on the one or more observations, that the palatal expander is not seated correctly in the upper palate of the patient.

Embodiment 21: The method of embodiment 20, wherein determining that the palatal expander is not seated correctly in the upper palate of the patient comprises: identifying a first edge of a retention attachment on at least one of a molar or premolar of the patient configured to engage with a receiving well of the palatal expander; identifying a second edge of the receiving well of the palatal expander; determining that a gap is present between the first edge and the second edge; and determining that the gap exceeds a gap threshold.

Embodiment 22: The method of embodiments 20-21, wherein determining that the palatal expander is not seated correctly in the upper palate of the patient comprises: identifying a surface of a tooth; identifying a surface of the palatal expander; and determining that a distance between the surface of the tooth and the surface of the palatal expander exceeds a threshold distance.

Embodiment 23: The method of embodiments 20-22, further comprising: sending a notification to a client device associated with the patient indicating that the palatal expander is not seated correctly.

Embodiment 24: The method of embodiments 1-23, wherein the one or more 2D images were generated during a retention phase of the treatment plan performed after palatal expansion, the method further comprising: determining, based on the one or more observations, whether an arch width has regressed or one or more molars have become tipped.

Embodiment 25: The method of embodiments 1-24, wherein the one or more observations comprise a metric characterizing at least one of an arch-width of the patient, a posterior cross-bite of a patient, a diastema of a patient, a palatine rugae of a patient, or a device fit of a palatal expander of the series of palatal expanders to the patient.

Embodiment 26: The method of embodiment 25, wherein determining a metric characterizing an arch-width of the patient comprises: measuring the arch-width in units of digital measurement; and converting the measured arch-width from units of digital measurement to units of physical measurement.

Embodiment 27: The method of embodiments 25-26, where determining an indication of an occurrence of a diastema comprises: segmenting the 2D images into a plurality of segments, wherein each segment of the plurality of segments corresponds to a tooth of the patient; and identifying a diastema based on a comparison of a spatial arrangement of the plurality of segments to a spatial arrangement of a previously segmented plurality of segments, wherein the previously segmented plurality of segments correspond to previously captured 2D images.

Embodiment 28: The method of embodiments 25-27, wherein determining a metric characterizing a palatine rugae of a patient, comprises: determining a longitudinal distance of a feature to a palatine rugae of the patient.

Embodiment 29: The method of embodiment 28, wherein determining a level of progress associated with the treatment plan comprises: determining a change in the longitudinal distance of the feature to the palatine rugae based on a comparison of the longitudinal distance of the feature to the palatine rugae to a previously determined longitudinal distance of the feature to the palatine rugae, wherein the previously determined longitudinal distance of the feature to the palatine rugae corresponds to one or more previously captured 2D images.

Embodiment 30: The method of embodiments 1-29, wherein the one or more observations comprise an indication of an occurrence of at least one of a soft tissue damage, an intrusion of supporting teeth, a buccal tipping of teeth, a tissue impingement, an eruption of teeth, or an exfoliation of teeth.

Embodiment 31: The method of embodiments 1-30, wherein the one or more observations comprise a metric characterizing at least one of an arch-width of the patient, a tipping of molars, or a discoloration of a palatal expander of the series of palatal expanders.

Embodiment 32: The method of embodiments 1-31, further comprising determining one or more actions to be performed based on the determined level of progress, the one or more actions comprising: advancing the patient to a subsequent treatment stage in the series of sequential treatment stages before a preplanned advancement time, or retaining the patient in a treatment current stage of the series of sequential treatment stages beyond the preplanned advancement time.

Embodiment 33: The method of embodiments 1-32, wherein the one or more 2D images comprises an image of an open mouth view of an upper palate of the patient, wherein determining the one or more observations comprises: measuring an arch width in units of digital measurement from the image of the open mouth view of the upper palate; converting the arch width from units of digital measurement to units of physical measurement; and comparing the arch width in the units of physical measurement to a target arch width of the treatment plan, wherein the one or more observations comprise at least one of the arch width in the units of physical measurement or a comparison result of the arch width in the units of physical measurement to the target arch width.

Embodiment 34: The method of embodiments 1-33, wherein processing the 2D images to determine one or more observations comprises: providing the one or more 2D images as input to one or more machine learning models; receiving, as output from the one or more machine learning models, a plurality of segments of the one or more 2D images, wherein each segment of the plurality of segments corresponds to a tooth of the patient; measuring a distance between a first segment and a second segment of the plurality of segments in units for digital measurement; and converting the distance from units for digital measurement into units for physical measurement.

Embodiment 35: The method of embodiments 1-34, wherein processing the 2D images to determine one or more observations comprises: estimating, from the one or more 2D images, an angle associated with a position of an image sensor used to capture the 2D images; computing a perspective correction factor based on the estimated angle; and modifying a measurement of a feature of the images based on the computed perspective correction factor.

Embodiment 36: The method of embodiments 1-35, further comprising: receiving an image of a current face of the patient; simulating a post-treatment face of the patient based at least in part on processing of the image of the current face of the patient; determining whether the post-treatment face of the patient is acceptable to the patient; and altering the treatment plan responsive to determining that the post-treatment face of the patient is not acceptable to the patient.

Embodiment 37: The method of embodiments 1-36, wherein at least one 2D image of the one or more 2D images of the oral cavity of the patient were taken while a current palatal expander in the series of palatal expanders was worn and shows the current palatal expander, the method further comprising: determining a level of wear of the current palatal expander based on processing of the at least one 2D image.

Embodiment 38: The method of embodiment 37, wherein determining the level of wear of the current palatal expander comprises processing the at least one 2D image using a trained machine learning model that outputs the level of wear of the current palatal expander.

Embodiment 39: The method of embodiments 37-38, further comprising: comparing the determined level of wear of the current palatal expander to a wear threshold; and outputting a notification responsive to determining that the determined level of wear of the current palatal expander exceeds the wear threshold.

Embodiment 40: A method for monitoring palatal expansion, the method comprising: receiving sensor data generated by one or more integrated sensors of a palatal expander manufactured for a patient; determining, via processing of the sensor data, one or more observations associated with the palatal expander; and providing a representation of progress of the palatal expansion based on the one or more observations.

Embodiment 41: The method of embodiment 40, wherein the one or more integrated sensors comprise at least one of a force sensor, a pressure sensor, a displacement sensor, a touch sensor, a colorimetric sensor, a biofilm sensor, or a temperature sensor.

Embodiment 42: The method of embodiments 40-41, wherein the one or more integrated sensors comprise one or more force sensors or pressure sensors that measure a transverse force or pressure on the palatal expander, the method further comprising: determining, based on the transverse force or pressure, a level of progress associated with a treatment plan, wherein one or more actions to be performed are determined based on the determined level of progress.

Embodiment 43: The method of embodiment 42, further comprising: determining that the transverse force or pressure exceeds an upper force or pressure threshold; and determining to retain the patient in a current stage of treatment longer than initially planned.

Embodiment 44: The method of embodiments 42-43, further comprising: determining that the transverse force or pressure is below a lower force or pressure threshold; and determining to advance the patient to a next stage of treatment earlier than initially planned.

Embodiment 45: The method of embodiment 44, wherein the one or more integrated sensors comprise at least one of a force sensor or a pressure sensor configured to measure at least one of a transverse force or a transverse pressure on the palatal expander, and wherein determining a level of progress associated with a treatment plan comprises determining whether at least one of the transverse force or the transverse pressure on the palatal expander satisfies one or more criteria for advancing the patient to a subsequent stage of the treatment plan.

Embodiment 46: The method of embodiment 45, further comprising: determining a force or pressure profile based on at least one of the transverse force or the transverse pressure as measured by a plurality of palatal expanders in a series of palatal expanders, wherein the one or more criteria comprise one or more force profile criteria or pressure profile criteria; wherein the force profile or pressure profile satisfies the one or more force profile criteria or pressure profile criteria based on the force profile showing at least one of a) a build-up of force or pressure or b) a current force or pressure that meets or exceeds a force threshold or pressure threshold.

Embodiment 47: The method of embodiments 45-46, further comprising: receiving additional sensor data generated by one or more integrated sensors of a retention device manufactured for the patient, wherein the retention device prevents palatal contraction after a series of palatal expanders have been worn by the patient, and wherein the additional sensor data comprises at least one of transverse force data or transverse pressure data; determining a force profile or pressure profile based on at least one of the transverse force data or the transverse pressure data as measured by the retention device over a period of time; determining whether the force profile or pressure profile satisfies one or more force profile criteria or pressure profile criteria; and at least one of ending a palatal expansion treatment or progressing to a next treatment responsive to determining that the force profile or pressure profile satisfies the one or more profile criteria.

Embodiment 48: The method of embodiment 47, wherein the one or more profile criteria comprise at least one of: a first criterion that measured force or pressure over time has been decreasing; or a second criterion that measured force or pressure is below a force threshold or pressure threshold.

Embodiment 49: The method of embodiments 40-48, wherein the one or more integrated sensors comprise a force sensor or pressure sensor in at least one of a buccal region or a lingual region of the palatal expander that measures a force or pressure applied on the palatal expander when the palatal expander is at least one of inserted onto an upper palate of the patient or removed from the upper palate of the patient, the method further comprising: determining whether the force or pressure applied on the palatal expander exceeds a threshold; and performing one or more actions responsive to determining that the force or pressure applied on the palatal expander exceeds the threshold.

Embodiment 50: The method of embodiment 49, wherein the one or more actions comprise generating a notification indicating at least one of proper insertion technique or proper removal technique for the palatal expander.

Embodiment 51: The method of embodiments 40-50, further comprising: generating a notification informing the patient of one or more actions to be performed; and sending the notification to a device associated with the patient.

Embodiment 52: The method of embodiment 51, wherein the device associated with the patient comprises at least one of a mobile device of the patient, a computing device of the patient, or a palatal expander container in possession of the patient.

Embodiment 53: The method of embodiments 40-52, wherein the one or more integrated sensors comprise a displacement sensor at an occlusal region of the palatal expander that measures displacement from a tooth of the patient, the method further comprising: determining whether the displacement exceeds a displacement threshold; and generating a notification indicating that the palatal expander is not fully seated on an upper palate of the patient responsive to determining that the displacement exceeds the displacement threshold.

Embodiment 54: The method of embodiments 40-53, wherein the one or more integrated sensors comprise a colorimetric sensor that measures a color change of the palatal expander, the method further comprising: determining whether the color change on the palatal expander exceeds a color change threshold; and generating a notification indicating that a consumption of materials is negatively affecting the palatal expander, responsive to determining that the color change exceeds the color change threshold.

Embodiment 55: The method of embodiments 40-54, wherein the one or more integrated sensors comprise a biofilm sensor that measures a biofilm build-up on the palatal expander, the method further comprising: determining whether the biofilm build-up on the palatal expander exceeds a biofilm build-up threshold; and generating a notification indicating that a consumption of materials is negatively affecting the palatal expander, responsive to determining that the biofilm build-up exceeds the biofilm build-up threshold.

Embodiment 56: The method of embodiments 40-55, wherein the one or more integrated sensors comprise a temperature sensor that measures a temperature of the palatal expander, the method further comprising: determining whether the temperature of the palatal expander exceeds a temperature threshold; and generating a notification indicating that a consumption of materials is negatively affecting the palatal expander, responsive to determining that the temperature exceeds the temperature threshold.

Embodiment 57: The method of embodiment 40, wherein the one or more integrated sensors comprise a contact sensor or a touch sensor at a palatal region of the palatal expander that measures contact with soft tissue of an upper palate of the patient, the method further comprising: determining whether the contact sensor or the touch sensor detects contact with the soft tissue of the upper palate; and determining to replace the palatal expander responsive to determining that the contact sensor or the touch sensor detects the contact with the soft tissue of the upper palate.

Embodiment 58: The method of embodiments 40-57, further comprising determining one or more actions to be performed, wherein the one or more actions to be performed comprise: advancing the patient to a subsequent treatment stage in a series of sequential treatment stages before a preplanned advancement time, or retaining the patient in a current treatment stage of the series of sequential treatment stages beyond the preplanned advancement time.

Embodiment 59: The method of embodiments 40-58, wherein the one or more integrated sensors comprise a first rotational sensor that measures a torque on a first region of the palatal expander.

Embodiment 60: The method of embodiment 59, wherein the first region is a first palatal region, and wherein the first rotational sensor measures a torque applied between the first palatal region of the palatal expander and a second palatal region of the palatal expander.

Embodiment 61: The method of embodiment 60, wherein the one or more integrated sensors comprise a second rotational sensor that measures a torque on a tooth region of the palatal expander.

Embodiment 62: The method of embodiments 40-61, wherein the one or more integrated sensors comprise one or more force sensors or one or more pressure sensors that measure a bite force or bite pressure, the method further comprising: determining one or more actions based on a measured bite force or bite pressure of the one or more force sensors or the one or more pressure sensors; and performing the one or more actions.

Embodiment 63: The method of embodiment 62, further comprising: counting a number of times that the patient bites the palatal expander based on measurements of the one or more force sensors or the one or more pressure sensors; determining whether the number of times that the patient bites the palatal expander exceeds a bite count threshold; and outputting a notification to replace the palatal expander responsive to determining that the number of times that the patient bites the palatal expander exceeds the bite count threshold, wherein the one or more actions comprise the notification.

Embodiment 64: The method of embodiments 62-63, further comprising: determining whether the measured bite force or bite pressure exceeds a bite force threshold or bite pressure threshold; and outputting a notification to replace the palatal expander responsive to determining that the measured bite force or bite pressure exceeds the bite force threshold or bite pressure threshold, wherein the one or more actions comprise the notification.

Embodiment 65: The method of embodiments 62-64, further comprising: identifying bruxism for the patient based on the measured bite force or bite pressure; and outputting a notification of the identified bruxism, wherein the one or more actions comprise the notification.

Embodiment 66: The method of embodiments 62-65, further comprising: determining a timing of when bite forces or bite pressures are measured; determining, based on the timing, that the patient is experiencing bruxism while sleeping; and outputting a notification of the bruxism, wherein the one or more actions comprise the notification.

Embodiment 67: The method of embodiments 40-66, wherein the one or more integrated sensors comprise a plurality of force sensors or a plurality of pressure sensors disposed at a plurality of regions of the palatal expander to measure bite force or bite pressure at the plurality of regions, the method further comprising: measuring bite force or bite pressure at the plurality of regions of the palatal expander using the plurality of force sensors or the plurality of pressure sensors; and performing a bite analysis based on the measured bite force or bite pressure at the plurality of regions of the palatal expander.

Embodiment 68: The method of embodiment 67, further comprising: determining one or more modifications to at least one of the palatal expander or one or more subsequent palatal expanders in a sequence of palatal expanders to be worn by the patient, wherein the one or more modifications improve bite contact points for at least one of the palatal expander or the one or more subsequent palatal expanders, wherein the one or more actions comprise determining the modifications.

Embodiment 69: The method of embodiment 68, wherein the one or more modifications comprise changing a thickness of one or more occlusal regions of at least one of the palatal expander or the one or more subsequent palatal expanders.

Embodiment 70: The method of embodiments 40-69, wherein determining the one or more observations comprises: determining an amount of time that the patient wears the palatal expander; and determining whether the amount of time satisfies a criterion.

Embodiment 71: The method of embodiment 70, further comprising: determining that the amount of time fails to satisfy a minimum wear time criterion; and outputting a notification for the patient to wear the palatal expander more frequently.

Embodiment 72: A method comprising: receiving one or more images of a current face of a patient at an intermediate stage of a dental treatment plan; generating one or more simulated images of a post-treatment face of the patient based at least in part on processing of the one or more images of the current face of the patient; determining one or more observations of the post-treatment face of the patient in the one or more simulated images; and determining, based on the one or more observations, whether to adjust the dental treatment plan.

Embodiment 73: The method of embodiment 72, wherein the dental treatment plan comprises at least one of a palatal expansion treatment plan or an orthodontic treatment plan.

Embodiment 74: The method of embodiments 72-73, wherein the one or more observations comprise at least one of a face width, a nasal breadth or a facial asymmetry.

Embodiment 75: The method of embodiments 72-74, wherein generating the one or more simulated images of the post-treatment face comprises processing an input comprising at least one of the one or more images of the current face of the patient or one or more prior images of a past face of the patient using a trained machine learning model, wherein the trained machine learning model outputs the one or more simulated images.

Embodiment 76: The method of embodiment 75, wherein the input to the trained machine learning model further comprises at least one of a current stage of treatment or a total number of stages of treatment for the dental treatment plan.

Embodiment 77: The method of embodiments 72-76, wherein determining the one or more observations comprises: processing the one or more simulated images to identify one or more facial features in the one or more simulated images; and performing one or more measurements of the one or more facial features.

Embodiment 78: The method of embodiment 77, wherein at least one of the processing of the one or more simulated images or the performing of the one or more measurements is performed using a trained machine learning model that receives the one or more simulated images as an input and outputs at least one of the one or more facial features or the one or more measurements.

Embodiment 79: The method of embodiments 72-78, wherein the dental treatment plan comprises a palatal expansion treatment plan, the method further comprising: determining that the post-treatment face has a predicted facial asymmetry; and updating the palatal expansion treatment plan by adjusting one or more palatal expanders in a sequence of palatal expanders to adjust one or more forces applied by the one or more palatal expanders to a palate of the patient to counter the predicted facial asymmetry.

Embodiment 80: The method of embodiment 79, wherein adjusting the one or more palatal expanders comprises modifying at least one of a rigidity, a thickness, or a material for one or more regions of the one or more palatal expanders.

Embodiment 81: The method of embodiments 72-80, wherein the dental treatment plan comprises a palatal expansion treatment plan, the method further comprising: determining that the post-treatment face has a predicted facial asymmetry; and outputting a recommendation to perform surgery to loosen one or more sutures of a palate of the patient to counteract the predicted facial asymmetry.

Embodiment 82: The method of embodiments 72-81, further comprising: annotating the one or more simulated images of the post-treatment face of the patient based on the one or more observations; and outputting the one or more simulated images to a display.

Embodiment 83: The method of embodiments 72-82, further comprising: determining that the post-treatment face of the patient is not acceptable to the patient; and stopping or shortening dental treatment.

Embodiment 84: A method for monitoring palatal expansion, the method comprising: accessing a treatment plan for a patient comprising a series of sequential treatment stages, each treatment stage associated with a particular palatal expander in a series of palatal expanders; capturing sensor data associated with the treatment plan from a palatal area of the patient; extracting, via processing of the sensor data, one or more observations associated with the treatment plan; determining, based on the one or more observations, a level of progress associated with the treatment plan; and providing a representation of progress based on the one or more observations.

Embodiment 85: The method of embodiment 84, wherein the sensor data comprises one or more two-dimensional (2D) images.

Embodiment 86: The method of embodiments 84-85, wherein the sensor data comprises data from an integrated sensor of a palatal expander of the series of palatal expanders.

Embodiment 87: The method of embodiments 84-86, wherein the sensor data comprises one or more two-dimensional (2D) images collected via an image sensor external to a palatal expander of the series of palatal expanders and additional sensor data collected from an integrated sensor of the palatal expander.

Embodiment 88: The method of embodiments 84-87, further comprising determining one or more actions to be performed based on the determined level of progress, wherein the one or more actions to be performed based on the determined level of progress comprise: advancing the patient to a subsequent treatment stage in the series of sequential treatment stages before a preplanned advancement time, or retaining the patient in a treatment current stage of the series of sequential treatment stages beyond the preplanned advancement time; and generating a notification indicating the one or more actions to be performed.

Embodiment 89: A computer readable medium comprising instructions that, when executed by a processing device, cause the processing device to perform the method of any of embodiments 1-88.

Embodiment 90: A system comprising: a memory; and a processing device operatively connected to the memory, the processing device configured to perform the method of any of embodiments 1-88.

Embodiment 91: A palatal expander comprising: a first tooth engagement region configured to secure the palatal expander to one or more first teeth of a patient; a second tooth engagement region configured to secure the palatal expander to one or more second teeth of the patient; a palatal region connecting the first tooth engagement region and the second tooth engagement region, the palatal region configured to apply a lateral force across a palate of a patient when the first tooth engagement region is secured to the one or more first teeth and the second tooth engagement region are secured to the one or more second teeth; and an integrated sensor configured to sense one or more properties associated with the palate of the patient.

Embodiment 92: The palatal expander of embodiment 91, further comprising: one or more additional sensors configured to detect whether the palatal expander is properly seated on retention attachments attached to at least one of the one or more first teeth or the one or more second teeth of the patient.

Embodiment 93: The palatal expander of embodiments 91-92, wherein the integrated sensor is disposed in a palatal region of the palatal expander and comprises a force sensor or pressure sensor to measure a transverse force or pressure on the palatal expander.

Embodiment 94: The palatal expander of embodiments 91-93, further comprising: an additional sensor disposed in at least one of a buccal region or a lingual region of the palatal expander, wherein the additional sensor is a force sensor configured to measure a force used to at least one of attach the palatal expander to an upper palate or remove the palatal expander from the upper palate.

Embodiment 95: The palatal expander of embodiments 91-94, further comprising: an additional sensor disposed in an occlusal region of the palatal expander, wherein the additional sensor is a displacement sensor configured to measure a force used to at least one of attach the palatal expander to an upper palate or remove the palatal expander from the upper palate.

Embodiment 96: The palatal expander of embodiments 91-95, further comprising: an additional sensor, wherein the additional sensor is a temperature sensor configured to measure a temperature of at least one of the palatal expander or an environment of the palatal expander.

Embodiment 97: The palatal expander of embodiments 91-96, further comprising: an additional sensor, wherein the additional sensor is a colorimetric sensor to measure staining of the palatal expander.

Embodiment 98: The palatal expander of embodiments 91-97, further comprising: an additional sensor, wherein the additional sensor is a biofilm sensor configured to measure a biofilm buildup on the palatal expander.

Embodiment 99: The palatal expander of embodiments 91-98, further comprising: a touch sensor in the palatal region, wherein the touch sensor is configured to detect contact with an upper palate.

Embodiment 100: The palatal expander of embodiments 91-99, further comprising: an additional sensor, wherein the additional sensor is a first rotational sensor configured to measure a torque on a first region of the palatal expander.

Embodiment 101: The palatal expander of embodiment 100, wherein the first region is a first palatal region, and wherein the first rotational sensor is configured to measure a torque applied between the first palatal region of the palatal expander and a second palatal region of the palatal expander.

Embodiment 102: The palatal expander of embodiment 101, further comprising: a second additional sensor, wherein the second additional sensor is a second rotational sensor configured to measure a torque on a tooth region of the palatal expander.

Embodiment 103: The palatal expander of embodiments 91-102, further comprising: one or more additional sensors at one or more regions of the palatal expander, wherein the one or more additional sensors comprise one or more force sensors or one or more pressure sensors configured to measure a bite force or bite pressure at the one or more regions of the palatal expander.

Any of the methods (including user interfaces) described herein can be implemented as software, hardware or firmware, and can be described as a non-transitory machine-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, computer models (e.g., for additive manufacturing) and instructions related to forming a dental device can be stored on a non-transitory machine-readable storage medium.

It should be understood that the above description is intended to be illustrative, and not restrictive. Many other embodiment examples will be apparent to those of skill in the art upon reading and understanding the above description. Although the present disclosure describes specific examples, it will be recognized that the systems and methods of the present disclosure are not limited to the examples described herein, but can be practiced with modifications within the scope of the appended claims. Accordingly, the specification and drawings are to be regarded in an illustrative sense rather than a restrictive sense. The scope of the present disclosure should, therefore, be determined with reference to the appended claims, along with the full scope of equivalents to which such claims are entitled.

The embodiments of methods, hardware, software, firmware, or code set forth above can be implemented via instructions or code stored on a machine-accessible, machine readable, computer accessible, or computer readable medium which are executable by a processing element. “Memory” includes any mechanism that provides (i.e., stores and/or transmits) information in a form readable by a machine, such as a computer or electronic system. For example, “memory” includes random-access memory (RAM), such as static RAM (SRAM) or dynamic RAM (DRAM); ROM; magnetic or optical storage medium; flash memory devices; electrical storage devices; optical storage devices; acoustical storage devices, and any type of tangible machine-readable medium suitable for storing or transmitting electronic instructions or information in a form readable by a machine (e.g., a computer).

Reference throughout this specification to “one embodiment” or “an embodiment” means that a particular feature, structure, or characteristic described in connection with the embodiment is included in at least one embodiment of the disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

In the foregoing specification, a detailed description has been given with reference to specific exemplary embodiments. It will, however, be evident that various modifications and changes can be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment, embodiment, and/or other exemplarily language does not necessarily refer to the same embodiment or the same example, but can refer to different and distinct embodiments, as well as potentially the same embodiment.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an embodiment” or “one embodiment” throughout is not intended to mean the same embodiment or embodiment unless described as such. Also, the terms “first,” “second,” “third,” “fourth,” etc. as used herein are meant as labels to distinguish among different elements and can not necessarily have an ordinal meaning according to their numerical designation.

A digital computer program, which can also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. The essential elements of a digital computer a central processing unit for performing or executing instructions and one or more memory devices for storing instructions and digital data. The central processing unit and the memory can be supplemented by, or incorporated in, special purpose logic circuitry or quantum simulators. Generally, a digital computer will also include, or be operatively coupled to receive digital data from or transfer digital data to, or both, one or more mass storage devices for storing digital data, e.g., magnetic, magneto-optical disks, optical disks, or systems suitable for storing information. However, a digital computer need not have such devices.

Digital computer-readable media suitable for storing digital computer program instructions and digital data include all forms of non-volatile digital memory, media, and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks.

Control of the various systems described in this specification, or portions of them, can be implemented in a digital computer program product that includes instructions that are stored on one or more non-transitory machine-readable storage media, and that are executable on one or more digital processing devices. The systems described in this specification, or portions of them, can each be implemented as an apparatus, method, or system that can include one or more digital processing devices and memory to store executable instructions to perform the operations described in this specification.

While this specification contains many specific embodiment details, these should not be construed as limitations on the scope of what can be claimed, but rather as descriptions of features that can be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing can be advantageous.

What is claimed is:

1. A method for monitoring palatal expansion, the method comprising:
accessing a treatment plan for a patient comprising a series of sequential treatment stages, each of a plurality of the series of sequential treatment stages associated with a particular palatal expander in a series of palatal expanders;
receiving one or more two-dimensional (2D) images of an oral cavity of the patient during a treatment stage of the treatment plan;
determining, via processing of the one or more 2D images, one or more observations of the oral cavity;
determining, based on the one or more observations, a level of progress associated with the treatment plan; and
providing a representation of progress corresponding to the determined level of progress.

2. The method of claim 1, wherein providing the representation of the progress comprises sending, to a client device, information configured to display the representation on a display of the client device.

3. The method of claim 1, further comprising:
determining a recommendation based on the determined level of progress; and
sending the recommendation to a client device, wherein the recommendation is a recommendation for an update to the treatment plan, the update determined based on the determined level of progress.

4. The method of claim 1, wherein the one or more observations comprise a metric characterizing a level of expansion achieved by the series of palatal expanders.

5. The method of claim 4, wherein determining the metric characterizing the level of expansion achieved by the palatal expander comprises:
determining a measurement of a feature in the one or more 2D images in units for digital image measurement; and
converting the measurement from the units for digital image measurement into units for physical measurement.

6. The method of claim 5, further comprising:
registering the one or more 2D images to at least one of an image or a three-dimensional (3D) model comprising one or more teeth of the patient that have known physical measurements; and
determining a conversion factor for converting between the units for digital image measurement and the units for physical measurement based on a result of the registering.

7. The method of claim 1, further comprising:
determining, based on the one or more observations, an indication of an occurrence of an adverse event associated with the oral cavity.

8. The method of claim 7, wherein determining an indication of an occurrence of an adverse event associated with the oral cavity comprises:
segmenting the one or more 2D images into a plurality of segments, wherein each segment of the plurality of segments corresponds to a tooth of the patient; and
determining a change in a spatial arrangement of the plurality of segments based on a comparison of the spatial arrangement of the plurality of segments to a spatial arrangement of a previously segmented plurality of segments or to an expected spatial arrangement of the plurality of segments for a current stage of treatment, wherein the previously segmented plurality of segments correspond to one or more previously captured images, and wherein the expected spatial arrangement is based on the treatment plan.

9. The method of claim 1, wherein the one or more 2D images comprise a 2D image of a palatal expander in an upper palate of the patient, the method further comprising:
determining, based on the one or more observations, that the palatal expander is not seated correctly in the upper palate of the patient.

10. The method of claim 9, wherein determining that the palatal expander is not seated correctly in the upper palate of the patient comprises:
identifying a first edge of a retention attachment on at least one of a molar or premolar of the patient configured to engage with a receiving well of the palatal expander;
identifying a second edge of the receiving well of the palatal expander;
determining that a gap is present between the first edge and the second edge; and
determining that the gap exceeds a gap threshold.

11. The method of claim 9, wherein determining that the palatal expander is not seated correctly in the upper palate of the patient comprises:
identifying a surface of a tooth;
identifying a surface of the palatal expander; and
determining that a distance between the surface of the tooth and the surface of the palatal expander exceeds a threshold distance.

12. The method of claim 1, wherein the one or more observations comprise a metric characterizing at least one of an arch-width of the patient, a posterior cross-bite of a patient, a diastema of a patient, a palatine rugae of a patient, or a device fit of a palatal expander of the series of palatal expanders to the patient.

13. The method of claim 12, wherein:
determining a metric characterizing a palatine rugae of a patient, comprises determining a longitudinal distance of a feature to a palatine rugae of the patient; and
determining a level of progress associated with the treatment plan comprises determining a change in the longitudinal distance of the feature to the palatine rugae based on a comparison of the longitudinal distance of the feature to the palatine rugae to a previously determined longitudinal distance of the feature to the palatine rugae, wherein the previously determined longitudinal distance of the feature to the palatine rugae corresponds to one or more previously captured 2D images.

14. The method of claim 1, further comprising determining one or more actions to be performed based on the determined level of progress, the one or more actions comprising:
advancing the patient to a subsequent treatment stage in the series of sequential treatment stages before a preplanned advancement time, or retaining the patient in a treatment current stage of the series of sequential treatment stages beyond the preplanned advancement time.

15. The method of claim 1, wherein processing the 2D images to determine one or more observations comprises:
providing the one or more 2D images as input to one or more machine learning models;
receiving, as output from the one or more machine learning models, a plurality of segments of the one or more 2D images, wherein each segment of the plurality of segments corresponds to a tooth of the patient;
measuring a distance between a first segment and a second segment of the plurality of segments in units for digital measurement; and
converting the distance from units for digital measurement into units for physical measurement.

16. The method of claim 1, further comprising:
receiving an image of a current face of the patient;
simulating a post-treatment face of the patient based at least in part on processing of the image of the current face of the patient;
determining whether the post-treatment face of the patient is acceptable to the patient; and
altering the treatment plan responsive to determining that the post-treatment face of the patient is not acceptable to the patient.

17. A method for monitoring palatal expansion, the method comprising:
receiving sensor data generated by one or more integrated sensors of a palatal expander manufactured for a patient;
determining, via processing of the sensor data, one or more observations associated with the palatal expander; and
providing a representation of progress of the palatal expansion based on the one or more observations.

18. The method of claim 17, wherein the one or more integrated sensors comprise one or more force or pressure sensors that measure a transverse force on the palatal expander, the method further comprising:
determining, based on the transverse force, a level of progress associated with a treatment plan, wherein one or more actions to be performed are determined based on the determined level of progress.

19. The method of claim 17, wherein the one or more integrated sensors comprise a plurality of force sensors or a plurality of pressure sensors disposed at a plurality of regions of the palatal expander to measure bite force at the plurality of regions, the method further comprising:
measuring bite force or bite pressure at the plurality of regions of the palatal expander using the plurality of force sensors or the plurality of pressure sensors;
performing a bite analysis based on the measured bite force or bite pressure at the plurality of regions of the palatal expander; and
outputting a result of the bite analysis to a display of an output device.

20. A palatal expander comprising:
a first tooth engagement region configured to secure the palatal expander to one or more first teeth of a patient;
a second tooth engagement region configured to secure the palatal expander to one or more second teeth of the patient;
a palatal region connecting the first tooth engagement region and the second tooth engagement region, the palatal region configured to apply a lateral force across a palate of a patient when the first tooth engagement region is secured to the one or more first teeth and the second tooth engagement region are secured to the one or more second teeth; and
an integrated sensor configured to sense one or more properties associated with the palate of the patient.
